(19) United States
(12) Patent Application Publication
(10) Pub. No.: US 2004/0005709 A1
(43) Pub. Date: Jan. 8, 2004

(54) Hybridization control of sequence variation

(76) Inventors: Henricus Renerus Jacobus Mattheus Hoogenboom, Maastricht (NL); Veerle Somers, Sint-Truiden (BE)

Correspondence Address: LOUIS MYERS, Fish & Richardson P.C., 225 Franklin Street, Boston, MA, 02110-2804, US

(21) Appl. No.: 10/279,633

(22) Filed: Oct. 24, 2002

Related U.S. Application Data
(60) Provisional application No. 60/343,954, filed on Oct. 24, 2001.

(51) Int. Cl. C12P 19/34; C12N 15/09; C12N 15/85

(52) U.S. Cl. 435/455; 435/91.2; 435/320.1

(57) ABSTRACT

Disclosed is a method of generating controlled mutations in a template nucleic acid sequence. Diverse oligonucleotides are hybridized to the template nucleic acid. The diverse oligonucleotides can have defined termini and length, and can be derived from a natural or synthetic source. Hybridization conditions are controlled to favor few or many mismatches between the diverse oligonucleotide and the template. Diversity strands encoding new variants are produced from the diverse oligonucleotides. The new variants that are generated can be screened for an improved property. In some implementations, the diverse oligonucleotides are derived from diverse nucleic acids by cleavage using that is directed by a cleavage-directing oligonucleotide.

### BACKGROUND

It is often desirable to improve the properties of a polypeptide. Nature evolves improved polypeptides by selecting for cells that perform better under selective circumstances. Random mutation of a nucleic acid sequence encoding a particular polypeptide produces polypeptide variants of that polypeptide. If the property of the particular polypeptide is improved to the extent that the improvement impacts the fitness of the cell or organism, selective pressures can result in selection and propagation of the improved variant.

Although many selection processes are very slow in Nature, at least on the order of years or centuries, some are rapid. One example of a rapid selection process is the selection of highly specific antibodies in the course of weeks after exposure to an antigen. This process, termed “antibody maturation,” entails the diversification of B cells that express an immunoglobulin that recognizes a non-self antigen. Immunological processes identify B cells that produce immunoglobulins with improved affinity and support the expansion of such productive B cells.

It is beneficial to employ methods that emulate the natural processes of improving polypeptides on a rapid time-scale and in a controlled laboratory environment. For example, many natural processes focus the scope of variation on residues likely to affect activity. Also, such natural processes frequently retain many of the initial residues that are dominant mediators of the activity. Thus, there is a need for a rapid method to access natural diversity present in vivo, e.g., the natural diversity of a population of somatically hypermutated antibody genes or the natural diversity present in homologous gene families within or between species.

### SUMMARY

The present invention provides a method for the introduction of diverse sequences into a template sequence at a defined position within the template and with a controlled degree of variability.

In one aspect, the invention features a method of forming a diversified strand. The method includes: a) providing i) a template nucleic acid strand and ii) diverse nucleic acids; b) annealing replicates of one or more cleavage-directing oligonucleotides to a plurality of members of the diverse nucleic acids to form cleavable regions; c) cleaving the cleavable regions to form a plurality of diverse oligonucleotides; d) contacting the plurality of diverse oligonucleotides and the template nucleic acid strand; and e) forming a diversified strand that incorporates an oligonucleotide of the plurality of diverse oligonucleotides and a segment of at least 10 (e.g., at least 50, 80, 120, 200) nucleotides complementary to the template nucleic acid strand.

In a preferred embodiment, the forming e) includes subjecting the contacted diverse oligonucleotides and the template nucleic acid strand to conditions such that only a subset of the plurality of diverse oligonucleotides can anneal to the template nucleic acid strand and extending and/or ligating an annealed oligonucleotide of the subset to form a diversified strand that is partially complementary to the template nucleic acid strand.

In one embodiment, the subjecting includes hybridizing diverse oligonucleotides of the subset and diverse oligonucleotides not of the subset to the template nucleic acid strand and washing the template nucleic acid strand, e.g., to dissociate the hybridized diverse oligonucleotides not of the subset. In another embodiment, the subjecting includes hybridizing diverse oligonucleotides of the subset, but not diverse oligonucleotides not of the subset.

In one embodiment, the cleavage-directing oligonucleotide includes a stem-loop structure, e.g., a structure that includes a recognition site for a Type IIS restriction enzyme. The cleaving is effected by the Type IIS restriction enzyme. In another embodiment, the cleaving is effected by a Type II restriction enzyme, e.g., an enzyme that recognizes a site of six basepairs, or less than six basepairs, e.g., five or four basepairs. In a preferred embodiment, the cleaving occurs at a temperature greater than 40° C.

In one embodiment, the cleavage-directing oligonucleotide forms a heteroduplex with the diverse nucleic acid and the cleavable region is fully complementary to the diverse nucleic acid within the heteroduplex. In a preferred embodiment, at least two cleavage-directing oligonucleotides are annealed to each of the diverse nucleic acids, e.g., one directs the cleavage of a 5′ terminus of a diverse oligonucleotide and the other directs the cleavage of a 3′ terminus of the diverse oligonucleotide. In a preferred embodiment, at least three pairs of cleavage-directing oligonucleotides are annealed. For example, the pairs can release at least one, two, or three diverse oligonucleotides. In one embodiment, the released diverse oligonucleotides encode one or more of: CDR1, CDR2, and CDR3 of an immunoglobulin variable domain (or a complement thereof). The diverse oligonucleotides can be released sequentially or concurrently.

In one embodiment, the diverse oligonucleotides include at least 10³, 10⁴, 10⁵, 10⁶, 10⁸, 10⁹, or 10¹⁰ different oligonucleotides. In one embodiment, each diverse oligonucleotide is less than 200, 120, 80, 70, 65, 60, 55, 50, 45, 40, or 35 nucleotides in length. The diverse oligonucleotides can be at least about 20, 25, 30, 35, 40, 45, 50, or 60 nucleotides in length. Each diverse oligonucleotide can be at least 40%, 50%, 60%, 70%, 80%, 90%, 95%, or 98% identical to at least another diverse oligonucleotide. For example, a diverse oligonucleotide can have 1, 2, 3, or at least 4 mismatches with respect to another diverse oligonucleotide.

In a preferred embodiment, each of the diverse oligonucleotides is of equal length as the others or is within 30, 20, 15, or 10% of the average length of the diverse oligonucleotides. In one embodiment, the diverse oligonucleotides of the plurality all have a length within 8, 6, 4, 3, 2, or 1 nucleotide of each other. Each of the diverse oligonucleotides can include 3′ and/or 5′ terminal regions of at least 6 nucleotides in length that are identical (or at least 70% identical) to corresponding terminal regions of each of the other diverse oligonucleotides. The terminal regions can be between 6 and 20 nucleotides in length, e.g., between 6 and 15, or 10 and 18 nucleotides in length. In a preferred embodiment, the terminal regions are exactly complementary to a corresponding site on the template nucleic acid. In a preferred embodiment, each of the diverse oligonucleotides includes a sequence corresponding to (e.g., partially complementary to) a common region of the template (e.g., of at least 5 or 10 nucleotides). Each diverse oligonucleotide can include a naturally occurring sequence or a synthetic sequence.

In a preferred embodiment, each diverse oligonucleotide encodes a CDR or fragment thereof, e.g., a fragment including at least 5 amino acids. In a much preferred embodiment, the diverse oligonucleotides further include 3′ and/or 5′ terminal regions that anneal to a sequence that flanks a sequence encoding a CDR (or its complement), e.g., a sequence that encodes a framework region (or its complement), e.g., at least one, two, three, four, or five nucleotides thereof. The terminal regions are preferably less varied than the sequence between the terminal regions among the diverse oligonucleotides. The CDR can be a heavy chain CDR (e.g., heavy chain CDR1, CDR2, and CDR3) or a light chain CDR (e.g., light chain CDR1, CDR2, and CDR3). In a preferred embodiment the diverse oligonucleotides preferably do not include the entire sequence of the framework regions which flank the CDR, e.g., contain less than 2, 5, 8, 10, or 15 of the amino acids of each of the flanking framework regions.

In another preferred embodiment, each diverse oligonucleotide encodes an enzyme active site residue, e.g., a residue that is within 2 Angstroms of a bound substrate or cofactor.

In one embodiment, the diverse nucleic acids include at least 10³, 10⁴, 10⁵, 10⁶, 10⁸, 10⁹, or 10¹⁰ different nucleic acids. The diverse nucleic acids can be, e.g., mRNA, cDNA, or genomic nucleic acids. Each diverse nucleic acid can be fixed to a solid support. In a preferred embodiment, the diverse nucleic acids are obtained from a mammalian cell, e.g., a hematopoietic cell such as a B or T cell. In another preferred embodiment, the mammalian cell is obtained from a subject having an immune disorder. The diverse nucleic acids can be obtained from a mammalian cell cultured in vitro. The cell can also be stimulated to undergo somatic mutagenesis of immunoglobulin genes, class switching of immunoglobulin genes, or proliferation. In one embodiment, the diverse nucleic acids are obtained from a cDNA pool from B cells, e.g., human B cells, e.g., from a subject afflicted with peripheral blood syndrome, vasculitis, an autoimmune disorder, or a neoplastic disorder. For example, the method can further include reverse transcribing cDNA from mRNA isolated from B cells.

The template nucleic acid can encode a polypeptide of at least 10, 20, 50, 100, or 200 amino acids. For example, the polypeptide can include a domain of a cell surface protein, an enzyme, a T cell receptor, an MHC protein, a protease inhibitor, a scaffold domain, or a transcription factor. In one embodiment, the polypeptide does not include an immunoglobulin domain, i.e., the polypeptide is not an antibody.

In one embodiment, the polypeptide has a binding activity or is preselected for a binding activity. In another embodiment, the polypeptide has an enzymatic activity or is preselected for an enzymatic activity. The polypeptide can be naturally occurring or synthetic, e.g., partially synthetic, e.g., a synthetic variant of a naturally occurring polypeptide. The preselecting can include identifying the polypeptide from a display library on the basis of the binding activity.

In a preferred embodiment, the polypeptide includes an immunoglobulin domain, e.g., a variable domain, e.g., a VH or VL domain. The sequence can further include an immunoglobulin constant domain, e.g., a CH1 or CL. With respect to a VH domain, the template can further include a sequence encoding a CH2 and CH3 domain. The VH or VL domain can include a synthetic CDR or a germline CDR (e.g., a human CDR). Further, the VH or VL domain can include a framework region, e.g., a human framework region. For example, the polypeptide can include a VH and CH1 domain or a VL and CL domain. The polypeptide can include both a VH and VL domain, e.g., as a single-chain Fv domain (ScFv). The polypeptide can be such that the VH and VL domains form, e.g., Fab fragments, F(ab′)₂, Fv fragments, and single-chain Fv fragments. The polypeptides include an antigen binding site, e.g., a functional antigen binding site. In a preferred embodiment the template includes at least one, and preferably two or three CDRs, and all or part of at least one framework region. For example, it can include at least one CDR, e.g., a CDR1, and all or part of the framework regions that flank CDR1.

In another preferred embodiment, the template nucleic acid encodes a second polypeptide. The first and second polypeptide can form a complex, e.g., the first and second polypeptide can be non-covalently bound or covalently bound, e.g., by one or more disulfides. For example, the complex can include a Fab.

In one embodiment the conditions for the contacting include a temperature greater than 40° C. The combining can include annealing at least some of the diverse oligonucleotides to the template nucleic acid strand. In another embodiment, the conditions include a temperature within 10 or 5° C. of a Tₘ, or a temperature greater than Tₘ-10°, Tₘ-5°, or Tₘ, wherein the Tₘ is the Tₘ of a segment of the template nucleic acid strand for its exact complement, and the segment is the region to which the diverse oligonucleotides hybridize. In one embodiment, the selected solution conditions are approximately a condition listed in Table 1. The hybridization conditions can include formamide or urea. The hybridization conditions can be selected so as to result in a preferred level of variation in the product, e.g., wherein the resulting molecules are at least 70, 80, 85, 90, 95, 97, or 98% homologous to the template. In some embodiments the level of homology is with regard to the entire length of the template, while in others it is with regard to the regions which correspond to diverse oligonucleotides.

In one embodiment, the template nucleic acid strand is limiting, and, for example, each diversity oligonucleotide of the population competes for the template nucleic acid strand under equilibrium binding conditions, e.g., conditions selected to favor competitive binding. In another embodiment, the template nucleic acid strand is not limiting. Exemplary molar ratios for the template nucleic acid strand to the diversity oligonucleotides include between 100:1 and 1:100; 10:1 and 1:10; 5:1 and 1:5; 10:1 and 1:1; 1:1 and 1:10.

In one embodiment, the subjecting includes separating at least some of the subset of diverse oligonucleotides that can anneal to the template nucleic acid strand from the remaining diverse oligonucleotides. The separating can include washing the template nucleic acid strand. The template nucleic acid can be attached to a solid support. For example, the template nucleic acid strand can be immobilized on a solid support, e.g., by a covalent or non-covalent linkage. The washing conditions can be more stringent than conditions for the contacting. In another embodiment, the separating includes a size separation, e.g., using a membrane porous to unannealed diverse oligonucleotides but not annealed diverse oligonucleotides, a gel exclusion method, a sedimentation method, or an electrophoretic method.

In a preferred embodiment, a plurality of template nucleic acid strands is provided. The template nucleic acid strands of the plurality can differ from one another. For example, the template nucleic acid strands can be at least 50% (e.g., at least 60%, 70%, or 80%) identical to each other. The template nucleic acid strands can encode polypeptides that share the same scaffold domain. In a preferred embodiment, each template nucleic acid strand of the plurality encodes a polypeptide that has an activity or is preselected for an activity.

For example, the providing of one or more template nucleic acids includes: (1) providing a display library, each member of which includes a nucleic acid that encodes a polypeptide and the encoded polypeptide; (2) identifying members of the display library for which the encoded polypeptide has at least a threshold activity; and (3) providing (e.g., isolating) template nucleic acid replicates for at least one of the identified members of the display library.

In one embodiment, each template strand or template strand complement encodes a polypeptide domain that, preferably, has at least a threshold activity. The method can further include screening the polypeptide encoded by the diversified strand complement (or a complement thereof), e.g., for an improved level of activity that exceeds the threshold activity. The threshold activity can be less than about 50, 10, 1, 0.1, or 0.01% of the improved level of activity. An exemplary activity that can be improved is affinity, e.g., Kₐ. In a preferred embodiment, the one or more template nucleic acid strands is a plurality of template nucleic acid strands. In one embodiment, each template nucleic acid strand of the plurality of template nucleic acid strands is the same. In another embodiment, the plurality of template nucleic-acid strands includes at least 2, 4, 8, 12, 30, 100, or 150 different template nucleic acid strands. In a preferred embodiment, the plurality of template nucleic acid strands includes different strands such that each or its complement encodes a polypeptide that includes a domain with at least a threshold activity of interest. For example, the strands of the plurality can include strands, each encoding a different polypeptide that is homologous (e.g., at least 40, 50, 60, 70, 80, 90, 95% identical) to the other encoded polypeptides, and/or has at least a threshold activity, e.g., a threshold measure of the same activity as the other polypeptides.

In another embodiment, the sequence of the template nucleic acid strand is not known at the time of the annealing. For example, the complete sequence of the template nucleic acid strand may be undetermined. More particularly, the sequence of the template nucleic acid strand in a region to which a diversity oligonucleotide can anneal is not known at the time of the annealing. An example of such a region is a region that encodes a CDR of an immunoglobulin variable domain.

The template strand can be linear or circular. It can be comprised of DNA, RNA, or combinations thereof. In one embodiment, the template strand is immobilized on a solid support, e.g., using a covalent or non-covalent linkage. The template strand can include uracil at at least some nucleotides. The template strand can further include a unique restriction enzyme site, one or more selectable markers, e.g., one functional selectable marker and one marker that includes a lesion, one or more bacteriophage genes, e.g., a gene encoding a major or minor coat protein, e.g., filamentous phage gene III. Each template nucleic acid can be tagged or fixed to a solid support. The template nucleic acid does not necessarily include regulatory sequences necessary for expression or even for encoding the entire protein, e.g., after alteration, the altered nucleic acid strands generated from the template can be modified to bring requisite sequences into an operable combination.

In another embodiment, the template nucleic strand includes a sequence encoding a transcription factor functional domain (e.g., for a two-hybrid assay), a cytotoxin, a label (e.g., green fluorescent protein or luciferase).

In one embodiment, the template strand comprises a promoter, e.g., a prokaryotic promoter, e.g., a bacteriophage promoter such as the T7, T3, or SP6 promoter. In another embodiment, the template strand includes a signal peptide, e.g., a eukaryotic or prokaryotic signal peptide.

In another embodiment, the template includes a nucleic acid sequence that encodes an enzyme or an inactivated enzyme, (e.g., as the sequence to be varied)

In one embodiment, the diversified nucleic acids are homologous (e.g., at least 30% homologous, more preferably at least about 40%, 50%, 60%, 70%, 80%, 90%, or more homologous) to one of the plurality of template nucleic acid strands. Preferably the diversified nucleic acids are homologous to each template nucleic acid strand of the plurality. In another embodiment, the diversified nucleic acids are homologous (e.g., at least 30% homologous, more preferably at least about 40%, 50%, 60%, 70%, or more homologous) to a reference domain, and each of the template nucleic acids is homologous to the reference domain.

In one embodiment, the annealed oligonucleotide is both extended and ligated. In another embodiment, the annealed oligonucleotide is extended. The extending and/or ligating can occur at least partially in a cell. Preferably, the extending and/or ligating occurs in vitro. The extending can be effected by a DNA polymerase or an RNA polymerase. Examples of DNA polymerases include E. coli polymerase I, T4 DNA polymerase, and reverse transcriptase (an RNA-dependent DNA polymerase). In a preferred embodiment, the DNA polymerase is a non-strand displacing DNA polymerase (e.g., T4 or T7 DNA polymerase). In another preferred embodiment, the DNA polymerase is a thermostable DNA polymerase. Another preferred DNA polymerase is the Klenow fragment of E. coli polymerase I or any DNA polymerase that lacks a 3′ to 5′ exonuclease activity.

In one embodiment, the method includes separating the diversified strand from the template strand. In another embodiment, the method includes separating diversified strand-template strand heteroduplexes from homoduplexes, e.g., using a mismatch binding protein. In another embodiment, the method can further include one or more of: amplifying the diversified strand, selectively disabling the template strand, and isolating the diversified strand.

The method can further include ligating the extended, hybridized diverse oligonucleotides. The method can include optionally introducing the diversified strand, a replicate, or complement thereof into cells, and/or optionally, translating the diversified strand, a replicate, or complement thereof.

In a preferred embodiment, the method further includes synthesizing a polypeptide encoded by the diversified strand or its complement. The translating can be in vitro or in vivo (i.e., in a host cell, e.g., a cultured cell or a transgenic cell that is part of an animal or plant). The host cell can be a prokaryotic cell (e.g., a bacterial cell) or is eukaryotic cell (e.g., a fungal cell, such as yeast, or a mammalian cell).

In one embodiment, the polypeptide is attached to the host cell surface (e.g., a yeast or mammalian cell surface, e.g., by means of a transmembrane protein or domain thereof or a peripheral membrane protein) or a virus surface, e.g., a filamentous phage coat protein or fragment thereof. The attachment can be direct or indirect (e.g., bridged), and can be covalent or non-covalent. In another embodiment, the polypeptide is attached to a solid support, e.g., a bead, particle, three-dimensional matrix, or planar array.

The method can further include constructing a library that includes the diversified strands, e.g., by introducing the diversified strand into a host cells with other diversified strands.

The method can further include screening the diversified strands or the complements thereof, e.g., using a method described herein. Exemplary methods include a display library, a polypeptide array, an in vitro assay, or an in vivo assay.

In another aspect, the invention features a method that includes: a) providing i) a template nucleic acid strand and ii) diverse nucleic acids; b) annealing a cleavage-directing oligonucleotide to a plurality of members of the diverse nucleic acids to form cleavable regions; c) cleaving the cleavable regions to form a plurality of diverse oligonucleotides; d) contacting the plurality of diverse oligonucleotides and the template nucleic acid strand in a mixture; e) subjecting the mixture to conditions such that only a subset of the plurality of diverse oligonucleotides can anneal to the template nucleic acid strand; and f) extending and/or ligating an annealed oligonucleotide of the subset to form a diversified strand that is partially complementary to the template nucleic acid strand.

In one embodiment, the cleavage-directing oligonucleotide includes a stem-loop structure, e.g., a structure that includes a recognition site for a Type IIS restriction enzyme. The cleaving is effected by the Type IIS restriction enzyme. In another embodiment, the cleaving is effected by a Type II restriction enzyme, e.g., an enzyme hat recognizes a site of six basepairs, or less than six basepairs, e.g., five or four basepairs. In a preferred embodiment, the cleaving occurs at a temperature greater than 40° C.

In one embodiment, the cleavage-directing oligonucleotide forms a heteroduplex with the diverse nucleic acid and the cleavable region is fully complementary to the diverse nucleic acid within the heteroduplex. In a preferred embodiment, at least two cleavage-directing oligonucleotides are annealed to each of the diverse nucleic acids, e.g., one directs the cleavage of a 5′ terminus of a diverse oligonucleotide and the other directs the cleavage of a 3′ terminus of the diverse oligonucleotide. In a preferred embodiment, at least three pairs of cleavage-directing oligonucleotides are annealed. For example, the pairs can release at least one, two, or three diverse oligonucleotides. In one embodiment, the released diverse oligonucleotides encode one or more of: CDR1, CDR2, and CDR3 of an immunoglobulin variable domain. The diverse oligonucleotides can be released sequentially or concurrently.

In one embodiment, the diverse oligonucleotides include at least 10³, 10⁴, 10⁵, 10⁶, 10⁸, 10⁹, or 10¹⁰ different oligonucleotides. In one embodiment, each diverse oligonucleotide is less than 200, 120, 80, 70, 65, 60, 55, 50, 45, 40, or 35 nucleotides in length. The diverse oligonucleotides can be at least about 20, 25, 30, 35, 40, 45, 50, or 60 nucleotides in length. Each diverse oligonucleotide can be at least 40%, 50%, 60%, 70%, 80%, 90%, 95%, or 98% identical to at least another diverse oligonucleotide. For example, a diverse oligonucleotide can have 1, 2, 3, or at least 4 mismatches with respect to another diverse oligonucleotide.

In a preferred embodiment, each of the diverse oligonucleotides is of equal length as the others or are within 30, 20, 15, or 10% of the average length of the diverse oligonucleotides. In one embodiment, the diverse oligonucleotides of the plurality all have a length within 8, 6, 4, 3, 2, or 1 nucleotide of each other. Each of the diverse oligonucleotides can include 3′ and/or 5′ terminal regions of at least 6 nucleotides in length that are identical (or at least 70% identical) to corresponding terminal regions of each of the other diverse oligonucleotides. The terminal regions can be between 6 and 20 nucleotides in length, e.g., between 6 and 15, or 10 and 18 nucleotides in length. In a preferred embodiment, the terminal regions are exactly complementary to a corresponding site on the template nucleic acid. In a preferred embodiment, each of the diverse oligonucleotides includes a sequence corresponding to (e.g., partially complementary to) a common region of the template (e.g., of at least 5 or 10 nucleotides). Each diverse oligonucleotide can include a naturally occurring sequence or a synthetic sequence.

In a preferred embodiment, each diverse oligonucleotide encodes a CDR or fragment thereof, e.g., a fragment including at least 5 amino acids. In a much preferred embodiment, the diverse oligonucleotides further include 3′ and/or 5′ terminal regions that anneal to a sequence that flanks a sequence encoding a CDR (or its complement), e.g., a sequence that encodes a framework region (or its complement), e.g., at least one, two, three, four, or five nucleotides thereof. The terminal regions are preferably less varied than the sequence between the terminal regions among the diverse oligonucleotides. The CDR can be a heavy chain CDR (e.g., heavy chain CDR1, CDR2, and CDR3) or a light chain CDR (e.g., light chain CDR1, CDR2, and CDR3). In a preferred embodiment the diverse oligonucleotides preferably do not include the entire sequence of the framework regions which flank the CDR, e.g., contain less than 2, 5, 8, 10, or 15 of the amino acids of each of the flanking framework regions.

In another preferred embodiment, each diverse oligonucleotide encodes an enzyme active site residue, e.g., a residue that is within 2 Angstroms of a bound substrate or cofactor.

In one embodiment, the diverse nucleic acids include at least 10³, 10⁴, 10⁵, 10⁶, 10⁸, 10⁹, or 10¹⁰ different nucleic acids. The diverse nucleic acids can be, e.g., mRNA, cDNA, or genomic nucleic acids. Each diverse nucleic acid can be fixed to a solid support. In a preferred embodiment, the diverse nucleic acids are obtained from a mammalian cell, e.g., a hematopoietic cell such as a B or T cell. In another preferred embodiment, the mammalian cell is obtained from a subject having an immune disorder. The diverse nucleic acids can be obtained from a mammalian cell cultured in vitro. The cell can also be stimulated to undergo somatic mutagenesis of immunoglobulin genes, class switching of immunoglobulin genes, or proliferation. In one embodiment, the diverse nucleic acids are obtained from a cDNA pool from B cells, e.g., human B cells, e.g., from a subject afflicted with peripheral blood syndrome, vasculitis, an autoimmune disorder, or a neoplastic disorder. For example, the method can further include reverse transcribing cDNA from mRNA isolated from B cells.

The template nucleic acid can encode a polypeptide of at least 10, 20, 50, 100, or 200 amino acids. For example, the polypeptide can include a domain of a cell surface protein, an enzyme, a T cell receptor, an MHC protein, a protease inhibitor, a scaffold domain, or a transcription factor. In one embodiment, the polypeptide does not include an immunoglobulin domain, i.e., the polypeptide is not an antibody.

In one embodiment, the polypeptide has a binding activity or is preselected for a binding activity. In another embodiment, the polypeptide has an enzymatic activity or is preselected for an enzymatic activity. The polypeptide can be naturally occurring or synthetic, e.g., partially synthetic, e.g., a synthetic variant of a naturally occurring polypeptide. The preselecting can include identifying the polypeptide from a display library on the basis of the binding activity.

In a preferred embodiment, the polypeptide includes an immunoglobulin domain, e.g., a variable domain, e.g., a VH or VL domain. The sequence can further include an immunoglobulin constant domain, e.g., a CH1 or CL. With respect to a VH domain, the template can further include a sequence encoding a CH2 and CH3 domain. The VH or VL domain can include a synthetic CDR or a germline CDR (e.g., a human CDR). Further, the VH or VL domain can include a framework region, e.g., a human framework region. For example, the polypeptide can include a VH and CH1 domain or a VL and CL domain. The polypeptide can include both a VH and VL domain, e.g., as a single-chain Fv domain (ScFv). The polypeptide can be such that the VH and VL domains form, e.g., Fab fragments, F(ab′)₂, Fv fragments, and single-chain Fv fragments. The polypeptides include an antigen binding site, e.g., a functional antigen binding site. In a preferred embodiment the template includes at least one, and preferably two or three CDRs, and all or part of at least one framework region. For example, it can include at least one CDR, e.g., a CDR1, and all or part of the framework regions that flank CDR1.

In another preferred embodiment, the template nucleic acid encodes a second polypeptide. The first and second polypeptide can form a complex, e.g., the first and second polypeptide can be non-covalently bound or covalently bound, e.g., by one or more disulfides. For example, the complex can include a Fab.

The combining can include annealing at least some of the diverse oligonucleotides to the template nucleic acid strand. In one embodiment, the annealed oligonucleotides include diverse oligonucleotides of the subset and diverse oligonucleotides not of the subset to the template nucleic acid strand. Subsequent washing of the template nucleic acid strand dissociates the hybridized diverse oligonucleotides not of the subset. In another embodiment, the annealed diverse oligonucleotides are exclusively from the subset.

In one embodiment the conditions for the contacting include a temperature greater than 40° C. In another embodiment, the conditions include a temperature within 10 or 5° C. of a Tₘ, or a temperature greater than Tₘ-10°, Tₘ-5°, or Tₘ, wherein the Tₘ is the Tₘ of a segment of the template nucleic acid strand for its exact complement, and the segment is the region to which the diverse oligonucleotides hybridize. In one embodiment, the selected solution conditions are approximately a condition listed in Table 1. The hybridization conditions can include formamide or urea. The hybridization conditions can be selected so as to result in a preferred level of variation in the product, e.g., wherein the resulting molecules are at least 70, 80, 85, 90, 95, 97, or 98% homologous to the template. In some embodiments the level of homology is with regard to the entire length of the template, while in others it is with regard to the regions which correspond to diverse oligonucleotides.

In one embodiment, the template nucleic acid strand is limiting, and, for example, each diversity oligonucleotide of the population competes for the template nucleic acid strand under equilibrium binding conditions, e.g., conditions selected to favor competitive binding. In another embodiment, the template nucleic acid strand is not limiting. Exemplary molar ratios for the template nucleic acid strand to the diversity oligonucleotides include between 100:1 and 1:100; 10:1 and 1:10; 5:1 and 1:5; 10:1 and 1:1; 1:1 and 1:10.

In one embodiment, the subjecting includes separating at least some of the subset of diverse oligonucleotides that can anneal to the template nucleic acid strand from the remaining diverse oligonucleotides. The separating can include washing the template nucleic acid strand. The template nucleic acid can be attached to a solid support. For example, the template nucleic acid strand can be immobilized on a solid support, e.g., by a covalent or non-covalent linkage. The washing conditions can be more stringent than conditions for the contacting. In another embodiment, the separating includes a size separation, e.g., using a membrane porous to unannealed diverse oligonucleotides but not annealed diverse oligonucleotides, a gel exclusion method, a sedimentation method, or an electrophoretic method.

In a preferred embodiment, a plurality of template nucleic acid strands is provided. The template nucleic acid strands of the plurality can differ from one another. For example, the template nucleic acid strands can be at least 50% (e.g., at least 60%, 70%, or 80%) identical to each other. The template nucleic acid strands can encode polypeptides that share the same scaffold domain. In a preferred embodiment, each template nucleic acid strand of the plurality encodes a polypeptide that has an activity or is preselected for an activity.

For example, the providing of one or more template nucleic acids includes: (1) providing a display library, each member of which includes a nucleic acid that encodes a polypeptide and the encoded polypeptide; (2) identifying members of the display library for which the encoded polypeptide has at least a threshold activity; and (3) providing (e.g., isolating) template nucleic acid replicates for at least one of the identified members of the display library.

In one embodiment, each template strand or template strand complement encodes a polypeptide domain that, preferably, has at least a threshold activity. The method can further include screening the polypeptide encoded by the diversified strand complement (or a complement thereof), e.g., for an improved level of activity that exceeds the threshold activity. The threshold activity can be less than about 50, 10, 1, 0.1, or 0.01% of the improved level of activity. In a preferred embodiment, the one or more template nucleic acid strands is a plurality of template nucleic acid strands. In one embodiment, each template nucleic acid strand of the plurality of template nucleic acid strands is the same. In another embodiment, the plurality of template nucleic acid strands includes at least 2, 4, 8, 12, 30, 100, or 150 different template nucleic acid strands. In a preferred embodiment, the plurality of template nucleic acid strands includes different strands such that each or its complement encodes a polypeptide that includes a domain with at least a threshold activity of interest. For example, the strands of the plurality can include strands, each encoding a different polypeptide that is homologous (e.g., at least 40, 50, 60, 70, 80, 90, 95%) to the other encoded polypeptides, and/or has at least a threshold activity, e.g., a threshold measure of the same activity as the other polypeptides.

In another embodiment, the sequence of the template nucleic acid strand is not known at the time of the annealing. For example, the complete sequence of the template nucleic acid strand may be undetermined. More particularly, the sequence of the template nucleic acid strand in a region to which a diversity oligonucleotide can anneal is not known at the time of the annealing. An example of such a region is a region that encodes a CDR of an immunoglobulin variable domain.

In a preferred embodiment, the template nucleic acid(s) comprise DNA. In another embodiment, they comprise RNA.

The template strand can be linear or circular. In one embodiment, the template strand is immobilized on a solid support, e.g., using a covalent or non-covalent linkage. The template strand can include uracil at at least some nucleotides. The template strand can further include a unique restriction enzyme site, one or more selectable markers, e.g., one functional selectable marker and one marker that includes a lesion, one or more bacteriophage genes, e.g., a gene encoding a major or minor coat protein, e.g., filamentous phage gene III. Each template nucleic acid can be tagged or fixed to a solid support.

In another embodiment, the template nucleic strand includes a sequence encoding a transcription factor functional domain (e.g., for a two-hybrid assay), a cytotoxin, a label (e.g., green fluorescent protein or luciferase).

In one embodiment, the template strand comprises a promoter, e.g., a prokaryotic promoter, e.g., a bacteriophage promoter such as the T7, T3, or SP6 promoter. In another embodiment, the template strand includes a signal peptide, e.g., a eukaryotic or prokaryotic signal peptide.

In another embodiment, the template includes a nucleic acid sequence that encodes an enzyme or an inactivated enzyme, (e.g., as the sequence to be varied)

In one embodiment, the diversified nucleic acids are homologous (e.g., at least 30% homologous, more preferably at least about 40%, 50%, 60%, 70%, 80%, 90%, or more homologous) to one of the plurality of template nucleic acid strands. Preferably the diversified nucleic acids are homologous to each template nucleic acid strand of the plurality. In another embodiment, the diversified nucleic acids are homologous (e.g., at least 30% homologous, more preferably at least about 40%, 50%, 60%, 70%, or more homologous) to a reference domain, and each of the template nucleic acids is homologous to the reference domain.

In one embodiment, the annealed oligonucleotide is both extended and ligated. In another embodiment, the annealed oligonucleotide is extended. The extending and/or ligating can occur at least partially in a cell. Preferably, the extending and/or ligating occurs in vitro. The extending can be effected by a DNA polymerase or an RNA polymerase. Examples of DNA polymerases include E. coli polymerase I, T4 DNA polymerase, and reverse transcriptase (an RNA-dependent DNA polymerase). In a preferred embodiment, the DNA polymerase is a non-strand displacing DNA polymerase (e.g., T4 or T7 DNA polymerase). In another preferred embodiment, the DNA polymerase is a thermostable DNA polymerase. Another preferred DNA polymerase is the Klenow fragment of E. coli polymerase I or any DNA polymerase that lacks a 3′ to 5′ exonuclease activity.

In one embodiment, the method includes separating the diversified strand from the template strand. In another embodiment, the method includes separating diversified strand-template strand heteroduplexes from homoduplexes, e.g., using a mismatch binding protein. In another embodiment, the method can further include one or more of: amplifying the diversified strand, selectively disabling the template strand, and isolating the diversified strand.

The method can further include ligating the extended, hybridized diverse oligonucleotides. The method can include optionally introducing the diversified strand, a replicate, or complement thereof into cells, and/or optionally, translating the diversified strand, a replicate, or complement thereof.

In a preferred embodiment, the method further includes synthesizing a polypeptide encoded by the diversified strand or its complement. The translating can be in vitro or in vivo (i.e., in a host cell, e.g., a cultured cell or a transgenic cell that is part of an animal or plant). The host cell can be a prokaryotic cell (e.g., a bacterial cell) or is eukaryotic cell (e.g., a fungal cell, such as yeast, or a mammalian cell).

In one embodiment, the polypeptide is attached to the host cell surface (e.g., a yeast or mammalian cell surface, e.g., by means of a transmembrane protein or domain thereof or a peripheral membrane protein) or a virus surface, e.g., a filamentous phage coat protein or fragment thereof. The attachment can be direct or indirect (e.g., bridged), and can be covalent or non-covalent. In another embodiment, the polypeptide is attached to a solid support, e.g., a bead, particle, three-dimensional matrix, or planar array.

The method can further include constructing a library that includes the diversified strands, e.g., by introducing the diversified strand into a host cells with other diversified strands.

The method can further include screening the diversified strands or the complements thereof, e.g., using a method described herein. Exemplary methods include a display library, a polypeptide array, an in vitro assay, or an in vivo assay.

In another aspect, the invention features a method that includes: a) providing i) a plurality of template nucleic acid strands and ii) diverse nucleic acids; b) annealing a cleavage-directing oligonucleotide to a plurality of members of the diverse nucleic acids to form cleavable regions; c) cleaving the cleavable regions to form a plurality of diverse oligonucleotides; d) combining the plurality of diverse oligonucleotides and the plurality of template nucleic acid strands in a mixture; e) subjecting the mixture to conditions such that only a subset of the plurality of diverse oligonucleotides can anneal to the template nucleic acid strands of the plurality of template nucleic acid strands; and f) for each template nucleic acid strand of the plurality, extending and/or ligating an annealed oligonucleotide of the subset to form a diversified strand that is partially complementary to the respective template nucleic acid strand. In a preferred embodiment, the method further includes: g) constructing a library of nucleic acids from the diversified strands formed from each template nucleic acid strand of the plurality.

The invention also features a library (e.g., a library of nucleic acids or polypeptides, or a display library) constructed by the method described above. A library of polypeptides can be arrayed. The display library can include members for which the diversified strand (or complement thereof) encodes a polypeptide that is attached to the nucleic acid. For example, the polypeptide can be attached to the coat of a bacteriophage. The polypeptide can be attached to a bacteriophage minor coat protein domain, e.g., the full-length gene III protein or the anchor domain of the gene III protein.

Referring again to the method featured above, the method can further include translating each diversified strand or a complement thereof. In a preferred embodiment, the template nucleic acid strands of the plurality differ from one another. For example, the template nucleic acid strands can be at least 50% (e.g., at least 60%, 70%, or 80%) identical to each other. The template nucleic acid strands can encode polypeptides that share the same scaffold domain. In a preferred embodiment, each template nucleic acid strand of the plurality encodes a polypeptide that has an activity or is preselected for an activity.

In a preferred embodiment, the plurality of template nucleic acids includes at least two (e.g., at least 10, 20, 50, 75, 100, or 250) different template nucleic acids and replicates thereof. The template nucleic acid strands of the plurality can differ from one another. For example, the template nucleic acid strands can be at least 50% (e.g., at least 60%, 70%, or 80%) identical to each other. The template nucleic acid strands can encode polypeptides that share the same scaffold domain. In a preferred embodiment, each template nucleic acid strand of the plurality encodes a polypeptide that has an activity or is preselected for an activity.

In one embodiment, the providing of a plurality template nucleic acids includes: (1) providing a display library, each member of which includes a nucleic acid that encodes a polypeptide and the encoded polypeptide; (2) identifying members of the display library for which the encoded polypeptide has at least a threshold activity; and (3) providing (e.g., isolating) template nucleic acid replicates for at least one of the identified members of the display library.

In one embodiment, each template strand or template strand complement encodes a polypeptide domain that, preferably, has at least a threshold activity. The method can further include screening the polypeptide encoded by the diversified strand complement (or a complement thereof), e.g., for an improved level of activity that exceeds the threshold activity. The threshold activity can be less than-about 50, 10, 1, 0.1, or 0.01% of the improved level of activity. In a preferred embodiment, the one or more template nucleic acid strands is a plurality of template nucleic acid strands. In one embodiment, each template nucleic acid strand of the plurality of template nucleic acid strands is the same. In another embodiment, the plurality of template nucleic acid strands includes at least 2, 4, 8, 12, 30, 100, or 150 different template nucleic acid strands. In a preferred embodiment, the plurality of template nucleic acid strands includes different strands such that each or its complement encodes a polypeptide that includes a domain with at least a threshold activity of interest. For example, the strands of the plurality can include strands, each encoding a different polypeptide that is homologous (e.g., at least 40, 50, 60, 70, 80, 90, 95%) to the other encoded polypeptides, and/or has at least a threshold activity, e.g., a threshold measure of the same activity as the other polypeptides.

In another embodiment, the sequence of the template nucleic acid strand is not known at the time of the annealing. For example, the complete sequence of the template nucleic acid strand may be undetermined. More particularly, the sequence of the template nucleic acid strand in a region to which a diversity oligonucleotide can anneal is not known at the time of the annealing. An example of such a region is a region that encodes a CDR of an immunoglobulin variable domain.

In a preferred embodiment, the template nucleic acid(s) comprise DNA. In another embodiment, they comprise RNA.

The template strand can be linear or circular. In one embodiment, the template strand is immobilized on a solid support, e.g., using a covalent or non-covalent linkage. The template strand can include uracil at at least some nucleotides. The template strand can further include a unique restriction enzyme site, one or more selectable markers, e.g., one functional selectable marker and one marker that includes a lesion, one or more bacteriophage genes, e.g., a gene encoding a major or minor coat protein, e.g., filamentous phage gene III. Each template nucleic acid can be tagged or fixed to a solid support.

In another embodiment, the template nucleic strand includes a sequence encoding a transcription factor functional domain (e.g., for a two-hybrid assay), a cytotoxin, a label (e.g., green fluorescent protein or luciferase).

In one embodiment, the template strand comprises a promoter, e.g., a prokaryotic promoter, e.g., a bacteriophage promoter such as the T7, T3, or SP6 promoter. In another embodiment, the template strand includes a signal peptide, e.g., a eukaryotic or prokaryotic signal peptide.

In another embodiment, the template includes a nucleic acid sequence that encodes an enzyme or an inactivated enzyme, (e.g., as the sequence to be varied).

In one embodiment, the cleavage-directing oligonucleotide includes a stem-loop structure, e.g., a structure that includes a recognition site for a Type IIS restriction enzyme. The cleaving is effected by the Type IIS restriction enzyme. In another embodiment, the cleaving is effected by a Type II restriction enzyme, e.g., an enzyme hat recognizes a site of six basepairs, or less than six basepairs, e.g., five or four basepairs. In a preferred embodiment, the cleaving occurs at a temperature greater than 40° C.

In one embodiment, the cleavage-directing oligonucleotide forms a heteroduplex with the diverse nucleic acid and the cleavable region is fully complementary to the diverse nucleic acid within the heteroduplex. In a preferred embodiment, at least two cleavage-directing oligonucleotides are annealed to each of the diverse nucleic acids, e.g., one directs the cleavage of a 5′ terminus of a diverse oligonucleotide and the other directs the cleavage of a 3′ terminus of the diverse oligonucleotide. In a preferred embodiment, at least three pairs of cleavage-directing oligonucleotides are annealed. For example, the pairs can release at least one, two, or three diverse oligonucleotides. In one embodiment, the released diverse oligonucleotides encode one or more of: CDR1, CDR2, and CDR3 of an immunoglobulin variable domain. The diverse oligonucleotides can be released sequentially or concurrently.

In one embodiment, the diverse oligonucleotides include at least 10³, 10⁴, 10⁵, 10⁶, 10⁸, 10⁹, or 10¹⁰ different oligonucleotides. In one embodiment, each diverse oligonucleotide is less than 200, 120, 80, 70, 65, 60, 55, 50, 45, 40, or 35 nucleotides in length. The diverse oligonucleotides can be at least about 20, 25, 30, 35, 40, 45, 50, or 60 nucleotides in length. Each diverse oligonucleotide can be at least 40%, 50%, 60%, 70%, 80%, 90%, 95%, or 98% identical to at least another diverse oligonucleotide. For example, a diverse oligonucleotide can have 1, 2, 3, or at least 4 mismatches with respect to another diverse oligonucleotide.

In a preferred embodiment, each of the diverse oligonucleotides is of equal length as the others or are within 30, 20, 15, or 10% of the average length of the diverse oligonucleotides. In one embodiment, the diverse oligonucleotides of the plurality all have a length within 8, 6, 4, 3, 2, or 1 nucleotide of each other. Each of the diverse oligonucleotides can include 3′ and/or 5′ terminal regions of at least 6 nucleotides in length that are identical (or at least 70% identical) to corresponding terminal regions of each of the other diverse oligonucleotides. The terminal regions can be between 6 and 20 nucleotides in length, e.g., between 6 and 15, or 10 and 18 nucleotides in length. In a preferred embodiment, the terminal regions are exactly complementary to a corresponding site on the template nucleic acid. In a preferred embodiment, each of the diverse oligonucleotides includes a sequence corresponding to (e.g., partially complementary to) a common region of the template (e.g., of at least 5 or 10 nucleotides). Each diverse oligonucleotide can include a naturally occurring sequence or a synthetic sequence.

In a preferred embodiment, each diverse oligonucleotide encodes a CDR or fragment thereof, e.g., a fragment including at least 5 amino acids. In a much preferred embodiment, the diverse oligonucleotides further include 3′ and/or 5′ terminal regions that anneal to a sequence that flanks a sequence encoding a CDR (or its complement), e.g., a sequence that encodes a framework region (or its complement), e.g., at least one, two, three, four, or five nucleotides thereof. The terminal regions are preferably less varied than the sequence between the terminal regions among the diverse oligonucleotides. The CDR can be a heavy chain CDR (e.g., heavy chain CDR1, CDR2, and CDR3) or a light chain CDR (e.g., light chain CDR1, CDR2, and CDR3). In a preferred embodiment the diverse oligonucleotides preferably do not include the entire sequence of the framework regions which flank the CDR, e.g., contain less than 2, 5, 8, 10, or 15 of the amino acids of each of the flanking framework regions.

In another preferred embodiment, each diverse oligonucleotide encodes an enzyme active site residue, e.g., a residue that is within 2 Angstroms of a bound substrate or cofactor.

In one embodiment, the diverse nucleic acids include at least 10³, 10⁴, 10⁵, 10⁶, 10⁸, 10⁹, or 10¹⁰ different nucleic acids. The diverse nucleic acids can be, e.g., mRNA, cDNA, or genomic nucleic acids. Each diverse nucleic acid can be fixed to a solid support. In a preferred embodiment, the diverse nucleic acids are obtained from a mammalian cell, e.g., a hematopoietic cell such as a B or T cell. In another preferred embodiment, the mammalian cell is obtained from a subject having an immune disorder. The diverse nucleic acids can be obtained from a mammalian cell cultured in vitro. The cell can also be stimulated to undergo somatic mutagenesis of immunoglobulin genes, class switching of immunoglobulin genes, or proliferation. In one embodiment, the diverse nucleic acids are obtained from a cDNA pool from B cells, e.g., human B cells, e.g., from a subject afflicted with peripheral blood syndrome, vasculitis, an autoimmune disorder, or a neoplastic disorder. For example, the method can further include reverse transcribing cDNA from mRNA isolated from B cells.

The template nucleic acid can encode a polypeptide of at least 10, 20, 50, 100, or 200 amino acids. For example, the polypeptide can include a domain of a cell surface protein, an enzyme, a T cell receptor, an MHC protein, a protease inhibitor, a scaffold domain, or a transcription factor. In one embodiment, the polypeptide does not include an immunoglobulin domain, i.e., the polypeptide is not an antibody.

In one embodiment, the polypeptide has a binding activity or is preselected for a binding activity. In another embodiment, the polypeptide has an enzymatic activity or is preselected for an enzymatic activity. The polypeptide can be naturally occurring or synthetic, e.g., partially synthetic, e.g., a synthetic variant of a naturally occurring polypeptide. The preselecting can include identifying the polypeptide from a display library on the basis of the binding activity.

In a preferred embodiment, the polypeptide includes an immunoglobulin domain, e.g., a variable domain, e.g., a VH or VL domain. The sequence can further include an immunoglobulin constant domain, e.g., a CH1 or CL. With respect to a VH domain, the template can further include a sequence encoding a CH2 and CH3 domain. The VH or VL domain can include a synthetic CDR or a germline CDR (e.g., a human CDR). Further, the VH or VL domain can include a framework region, e.g., a human framework region. For example, the polypeptide can include a VH and CH1 domain or a VL and CL domain. The polypeptide can include both a VH and VL domain, e.g., as a single-chain Fv domain (ScFv). The polypeptide can be such that the VH and VL domains form, e.g., Fab fragments, F(ab′)₂, Fv fragments, and single-chain Fv fragments. The polypeptides include an antigen binding site, e.g., a functional antigen binding site. In a preferred embodiment the template includes at least one, and preferably two or three CDRs, and all or part of at least one framework region. For example, it can include at least one CDR, e.g., a CDR1, and all or part of the framework regions that flank CDR1.

In another preferred embodiment, the template nucleic acid encodes a second polypeptide. The first and second polypeptide can form a complex, e.g., the first and second polypeptide can be non-covalently bound or covalently bound, e.g., by one or more disulfides. For example, the complex can include a Fab.

The combining can include annealing at least some of the diverse oligonucleotides to the template nucleic acid strand. In one embodiment, the annealed oligonucleotides include diverse oligonucleotides of the subset and diverse oligonucleotides not of the subset to the template nucleic acid strand. Subsequent washing of the template nucleic acid strand dissociates the hybridized diverse oligonucleotides not of the subset. In another embodiment, the annealed diverse oligonucleotides are exclusively from the subset.

In one embodiment the conditions for the contacting include a temperature greater than 40° C. In another embodiment, the conditions include a temperature within 10 or 5° C. of a Tₘ, or a temperature greater than Tₘ-10°, Tₘ-5°, or Tₘ, wherein the Tₘ is the Tₘ of a segment of the template nucleic acid strand for its exact complement, and the segment is the region to which the diverse oligonucleotides hybridize. In one embodiment, the selected solution conditions are approximately a condition listed in Table 1. The hybridization conditions can include formamide or urea. The hybridization conditions can be selected so as to result in a preferred level of variation in the product, e.g., wherein the resulting molecules are at least 70, 80, 85, 90, 95, 97, or 98% homologous to the template. In some embodiments the level of homology is with regard to the entire length of the template, while in others it is with regard to the regions which correspond to diverse oligonucleotides.

In one embodiment, the template nucleic acid strand is limiting, and, for example, each diversity oligonucleotide of the population competes for the template nucleic acid strand under equilibrium binding conditions, e.g., conditions selected to favor competitive binding. In another embodiment, the template nucleic acid strand is not limiting. Exemplary molar ratios for the template nucleic acid strand to the diversity oligonucleotides include between 100:1 and 1:100; 10:1 and 1:10; 5:1 and 1:5; 10:1 and 1:1; 1:1 and 1:10.

In one embodiment, the subjecting includes separating at least some of the subset of diverse oligonucleotides that can anneal to the template nucleic acid strand from the remaining diverse oligonucleotides. The separating can include washing the template nucleic acid strand. The template nucleic acid can be attached to a solid support. For example, the template nucleic acid strand can be immobilized on a solid support, e.g., by a covalent or non-covalent linkage. The washing conditions can be more stringent than conditions for the contacting. In another embodiment, the separating includes a size separation, e.g., using a membrane porous to unannealed diverse oligonucleotides but not annealed diverse oligonucleotides, a gel exclusion method, a sedimentation method, or an electrophoretic method.

In one embodiment, the diversified nucleic acids are homologous (e.g., at least 30% homologous, more preferably at least about 40%, 50%, 60%, 70%, 80%, 90%, or more homologous) to one of the plurality of template nucleic acid strands. Preferably the diversified nucleic acids are homologous to each template nucleic acid strand of the plurality. In another embodiment, the diversified nucleic acids are homologous (e.g., at least 30% homologous, more preferably at least about 40%, 50%, 60%, 70%, or more homologous) to a reference domain, and each of the template nucleic acids is homologous to the reference domain.

In one embodiment, the annealed oligonucleotide is both extended and ligated. In another embodiment, the annealed oligonucleotide is extended. The extending and/or ligating can occur at least partially in a cell. Preferably, the extending and/or ligating occurs in vitro. The extending can be effected by a DNA polymerase or an RNA polymerase. Examples of DNA polymerases include E. coli polymerase I, T4 DNA polymerase, and reverse transcriptase (an RNA-dependent DNA polymerase). In a preferred embodiment, the DNA polymerase is a non-strand displacing DNA polymerase (e.g., T4 or T7 DNA polymerase). In another preferred embodiment, the DNA polymerase is a thermostable DNA polymerase. Another preferred DNA polymerase is the Klenow fragment of E. coli polymerase I or any DNA polymerase that lacks a 3′ to 5′ exonuclease activity.

In one embodiment, the method includes separating the diversified strand from the template strand. In another embodiment, the method includes separating diversified strand-template strand heteroduplexes from homoduplexes, e.g., using a mismatch binding protein. In another embodiment, the method can further include one or more of: amplifying the diversified strand, selectively disabling the template strand, and isolating the diversified strand.

The method can further include ligating the extended, hybridized diverse oligonucleotides. The method can include optionally introducing the diversified strand, a replicate, or complement thereof into cells, and/or optionally, translating the diversified strand, a replicate, or complement thereof.

In a preferred embodiment, the method further includes synthesizing a polypeptide encoded by the diversified strand or its complement. The translating can be in vitro or in vivo (i.e., in a host cell, e.g., a cultured cell or a transgenic cell that is part of an animal or plant). The host cell can be a prokaryotic cell (e.g., a bacterial cell) or is eukaryotic cell (e.g., a fungal cell, such as yeast, or a mammalian cell).

In one embodiment, the polypeptide is attached to the host cell surface (e.g., a yeast or mammalian cell surface, e.g., by means of a transmembrane protein or domain thereof or a peripheral membrane protein) or a virus surface, e.g., a filamentous phage coat protein or fragment thereof. The attachment can be direct or indirect (e.g., bridged), and can be covalent or non-covalent. In another embodiment, the polypeptide is attached to a solid support, e.g., a bead, particle, three-dimensional matrix, or planar array.

The method can further include constructing a library that includes the diversified strands, e.g., by introducing the diversified strand into a host cells with other diversified strands.

The method can further include screening the diversified strands or the complements thereof, e.g., using a method described herein. Exemplary methods include a display library, a polypeptide array, an in vitro assay, or an in vivo assay.

In another aspect, the invention features a method that include: a) providing i) a template nucleic acid strand and ii) a plurality of diverse oligonucleotides; b) contacting the plurality of diverse oligonucleotides and the template nucleic acid strand in a mixture; c) subjecting the mixture to conditions such that only a subset of the plurality of diverse oligonucleotides can anneal to the template nucleic acid strand; d) separating at least the diverse oligonucleotides not in the subset from the mixture; and e) extending and/or ligating an annealed oligonucleotide of the subset to form a diversified strand that is partially complementary to the template nucleic acid strand.

The separating can include washing the template nucleic acid strand. For example, the template nucleic acid strand can be immobilized on a solid support, e.g., by a covalent or non-covalent linkage. The washing conditions can be more stringent than conditions for the contacting. In another embodiment, the separating includes a size separation, e.g., using a membrane porous to unannealed diverse oligonucleotides but not annealed diverse oligonucleotides, a gel exclusion method, a sedimentation method, or an electrophoretic method.

In one embodiment, the providing of diverse oligonucleotides includes: providing a plurality of diverse nucleic acids; annealing at least a first pair of cleavage-directing oligonucleotides to a given strand of each diverse nucleic acid of the plurality to form cleavable regions for each given strand; and cleaving the cleavable regions of each given strand to yield at least the plurality of diverse oligonucleotides from the given strands, each diverse oligonucleotide being unique in the plurality of diverse oligonucleotides. In a preferred embodiment, the diverse nucleic acids are obtained from a cDNA pool from B cells, e.g., human B cells, e.g., from a subject afflicted with peripheral blood syndrome, vasculitis, an autoimmune disorder, or a neoplastic disorder. For example, the method can further include reverse transcribing cDNA from mRNA isolated from B cells.

In one embodiment, the cleavage-directing oligonucleotide includes a stem-loop structure, e.g., a structure that includes a recognition site for a Type IIS restriction enzyme. The cleaving is effected by the Type IIS restriction enzyme. In another embodiment, the cleaving is effected by a Type II restriction enzyme, e.g., an enzyme hat recognizes a site of six basepairs, or less than six basepairs, e.g., five or four basepairs. In a preferred embodiment, the cleaving occurs at a temperature greater than 40° C.

In one embodiment, the cleavage-directing oligonucleotide forms a heteroduplex with the diverse nucleic acid and the cleavable region is fully complementary to the diverse nucleic acid within the heteroduplex. In a preferred embodiment, at least two cleavage-directing oligonucleotides are annealed to each of the diverse nucleic acids, e.g., one directs the cleavage of a 5′ terminus of a diverse oligonucleotide and the other directs the cleavage of a 3′ terminus of the diverse oligonucleotide. In a preferred embodiment, at least three pairs of cleavage-directing oligonucleotides are annealed. For example, the pairs can release at least one, two, or three diverse oligonucleotides. In one embodiment, the released diverse oligonucleotides encode one or more of: CDR1, CDR2, and CDR3 of an immunoglobulin variable domain. The diverse oligonucleotides can be released sequentially or concurrently.

In one embodiment, the diverse oligonucleotides include at least 10³, 10⁴, 10⁵, 10⁶, 10⁸, 10⁹, or 10¹⁰ different oligonucleotides. In one embodiment, each diverse oligonucleotide is less than 200, 120, 80, 70, 65, 60, 55, 50, 45, 40, or 35 nucleotides in length. The diverse oligonucleotides can be at least about 20, 25, 30, 35, 40, 45, 50, or 60 nucleotides in length. Each diverse oligonucleotide can be at least 40%, 50%, 60%, 70%, 80%, 90%, 95%, or 98% identical to at least another diverse oligonucleotide. For example, a diverse oligonucleotide can have 1, 2, 3, or at least 4 mismatches with respect to another diverse oligonucleotide.

In a preferred embodiment, each of the diverse oligonucleotides is of equal length as the others or are within 30, 20, 15, or 10% of the average length of the diverse oligonucleotides. In one embodiment, the diverse oligonucleotides of the plurality all have a length within 8, 6, 4, 3, 2, or 1 nucleotide of each other. Each of the diverse oligonucleotides can include 3′ and/or 5′ terminal regions of at least 6 nucleotides in length that are identical (or at least 70% identical) to corresponding terminal regions of each of the other diverse oligonucleotides. The terminal regions can be between 6 and 20 nucleotides in length, e.g., between 6 and 15, or 10 and 18 nucleotides in length. In a preferred embodiment, the terminal regions are exactly complementary to a corresponding site on the template nucleic acid. In a preferred embodiment, each of the diverse oligonucleotides includes a sequence corresponding to (e.g., partially complementary to) a common region of the template (e.g., of at least 5 or 10 nucleotides). Each diverse oligonucleotide can include a naturally occurring sequence or a synthetic sequence. The diverse oligonucleotides can be constructed by chemical synthesis. In another embodiment, the diverse oligonucleotides are constructed by cleavage of a diverse nucleic acid strand.

In a preferred embodiment, each diverse oligonucleotide encodes a CDR or fragment thereof, e.g., a fragment including at least 5 amino acids. In a much preferred embodiment, the diverse oligonucleotides further include 3′ and/or 5′ terminal regions that anneal to a sequence that flanks a sequence encoding a CDR (or its complement), e.g., a sequence that encodes a framework region (or its complement), e.g., at least one, two, three, four, or five nucleotides thereof. The terminal regions are preferably less varied than the sequence between the terminal regions among the diverse oligonucleotides. The CDR can be a heavy chain CDR (e.g., heavy chain CDR1, CDR2, and CDR3) or a light chain CDR (e.g., light chain CDR1, CDR2, and CDR3). In a preferred embodiment the diverse oligonucleotides preferably do not include the entire sequence of the framework regions which flank the CDR, e.g., contain less than 2, 5, 8, 10, or 15 of the amino acids of each of the flanking framework regions.

In another preferred embodiment, each diverse oligonucleotide encodes an enzyme active site residue, e.g., a residue that is within 2 Angstroms of a bound substrate or cofactor.

In one embodiment, the diverse nucleic acids include at least 10³, 10⁴, 10⁵, 10⁶, 10⁸, 10⁹, or 10¹⁰ different nucleic acids. The diverse nucleic acids can be, e.g., mRNA, cDNA, or genomic nucleic acids. Each diverse nucleic acid can be fixed to a solid support. In a preferred embodiment, the diverse nucleic acids are obtained from a mammalian cell, e.g., a hematopoietic cell such as a B or T cell. In another preferred embodiment, the mammalian cell is obtained from a subject having an immune disorder. The diverse nucleic acids can be obtained from a mammalian cell cultured in vitro. The cell can also be stimulated to undergo somatic mutagenesis of immunoglobulin genes, class switching of immunoglobulin genes, or proliferation.

The template nucleic acid can encode a polypeptide of at least 10, 20, 50, 100, or 200 amino acids. For example, the polypeptide can include a domain of a cell surface protein, an enzyme, a T cell receptor, an MHC protein, a protease inhibitor, a scaffold domain, or a transcription factor. In one embodiment, the polypeptide does not include an immunoglobulin domain, i.e., the polypeptide is not an antibody.

In one embodiment, the polypeptide has a binding activity or is preselected for a binding activity. In another embodiment, the polypeptide has an enzymatic activity or is preselected for an enzymatic activity. The polypeptide can be naturally occurring or synthetic, e.g., partially synthetic, e.g., a synthetic variant of a naturally occurring polypeptide. The preselecting can include identifying the polypeptide from a display library on the basis of the binding activity.

In a preferred embodiment, the polypeptide includes an immunoglobulin domain, e.g., a variable domain, e.g., a VH or VL domain. The sequence can further include an immunoglobulin constant domain, e.g., a CH1 or CL. With respect to a VH domain, the template can further include a sequence encoding a CH2 and CH3 domain. The VH or VL domain can include a synthetic CDR or a germline CDR (e.g., a human CDR). Further, the VH or VL domain can include a framework region, e.g., a human framework region. For example, the polypeptide can include a VH and CH1 domain or a VL and CL domain. The polypeptide can include both a VH and VL domain, e.g., as a single-chain Fv domain (ScFv). The polypeptide can be such that the VH and VL domains form, e.g., Fab fragments, F(ab′)₂, Fv fragments, and single-chain Fv fragments. The polypeptides include an antigen binding site, e.g., a functional antigen binding site. In a preferred embodiment the template includes at least one, and preferably two or three CDRs, and all or part of at least one framework region. For example, it can include at least one CDR, e.g., a CDR1, and all or part of the framework regions which flank CDR1.

In another preferred embodiment, the template nucleic acid encodes a second polypeptide. The first and second polypeptide can form a complex, e.g., the first and second polypeptide can be non-covalently bound or covalently bound, e.g., by one or more disulfides. For example, the complex can include a Fab.

The combining can include annealing at least some of the diverse oligonucleotides to the template nucleic acid strand. In one embodiment, the annealed oligonucleotides include diverse oligonucleotides of the subset and diverse oligonucleotides not of the subset to the template nucleic acid strand. Subsequent washing of the template nucleic acid strand dissociates the hybridized diverse oligonucleotides not of the subset. In another embodiment, the annealed diverse oligonucleotides are exclusively from the subset.

In one embodiment the conditions for the contacting include a temperature greater than 40° C. In another embodiment, the conditions include a temperature within 10 or 5° C. of a Tₘ, or a temperature greater than Tₘ-10°, Tₘ-5°, or Tₘ, wherein the Tₘ is the Tₘ of a segment of the template nucleic acid strand for its exact complement, and the segment is the region to which the diverse oligonucleotides hybridize. In one embodiment, the selected solution conditions are approximately a condition listed in Table 1. The hybridization conditions can include formamide or urea. The hybridization conditions can be selected so as to result in a preferred level of variation in the product, e.g., wherein the resulting molecules are at least 70, 80, 85, 90, 95, 97, or 98% homologous to the template. In some embodiments the level of homology is with regard to the entire length of the template, while in others it is with regard to the regions which correspond to diverse oligonucleotides.

In one embodiment, the template nucleic acid strand is limiting, and, for example, each diversity oligonucleotide of the population competes for the template nucleic acid strand under equilibrium binding conditions, e.g., conditions selected to favor competitive binding. In another embodiment, the template nucleic acid strand is not limiting. Exemplary molar ratios for the template nucleic acid strand to the diversity oligonucleotides include between 100:1 and 1:100; 10:1 and 1:10; 5:1 and 1:5; 10:1 and 1:1; 1:1 and 1:10.

In a preferred embodiment, a plurality of template nucleic acid strands are provided. The template nucleic acid strands of the plurality can differ from one another. For example, the template nucleic acid strands can be at least 50% (e.g., at least 60%, 70%, or 80%) identical to each other. The template nucleic acid strands can encode polypeptides that share the same scaffold domain. In a preferred embodiment, each template nucleic acid strand of the plurality encodes a polypeptide that has an activity or is preselected for an activity.

For example, the providing of one or more template nucleic acids includes: (1) providing a display library, each member of which includes a nucleic acid that encodes a polypeptide and the encoded polypeptide; (2) identifying members of the display library for which the encoded polypeptide has at least a threshold activity; and (3) providing (e.g., isolating) template nucleic acid replicates for at least one of the identified members of the display library.

In one embodiment, each template strand or template strand complement encodes a polypeptide domain that, preferably, has at least a threshold activity. The method can further include screening the polypeptide encoded by the diversified strand complement (or a complement thereof), e.g., for an improved level of activity that exceeds the threshold activity. The threshold activity can be less than about 50, 10, 1, 0.1, or 0.01% of the improved level of activity. In a preferred embodiment, the one or more template nucleic acid strands is a plurality of template nucleic acid strands. In one embodiment, each template nucleic acid strand of the plurality of template nucleic acid strands is the same. In another embodiment, the plurality of template nucleic acid strands includes at least 2, 4, 8, 12, 30, 100, or 150 different template nucleic acid strands. In a preferred embodiment, the plurality of template nucleic acid strands includes different strands such that each or its complement encodes a polypeptide that includes a domain with at least a threshold activity of interest. For example, the strands of the plurality can include strands, each encoding a different polypeptide that is homologous (e.g., at least 40, 50, 60, 70, 80, 90, 95%) to the other encoded polypeptides, and/or has at least a threshold activity, e.g., a threshold measure of the same activity as the other polypeptides.

In another embodiment, the sequence of the template nucleic acid strand is not known at the time of the annealing. For example, the complete sequence of the template nucleic acid strand may be undetermined. More particularly, the sequence of the template nucleic acid strand in a region to which a diversity oligonucleotide can anneal is not known at the time of the annealing. An example of such a region is a region that encodes a CDR of an immunoglobulin variable domain.

In a preferred embodiment, the template nucleic acid(s) comprise DNA. In another embodiment, they comprise RNA.

The template strand can be linear or circular. In one embodiment, the template strand is immobilized on a solid support, e.g., using a covalent or non-covalent linkage. The template strand can include uracil at at least some nucleotides. The template strand can further include a unique restriction enzyme site, one or more selectable markers, e.g., one functional selectable marker and one marker that includes a lesion, one or more bacteriophage genes, e.g., a gene encoding a major or minor coat protein, e.g., filamentous phage gene III. Each template nucleic acid can be tagged or fixed to a solid support.

In another embodiment, the template nucleic strand includes a sequence encoding a transcription factor functional domain (e.g., for a two-hybrid assay), a cytotoxin, a label (e.g., green fluorescent protein or luciferase).

In one embodiment, the template strand comprises a promoter, e.g., a prokaryotic promoter, e.g., a bacteriophage promoter such as the T7, T3, or SP6 promoter. In another embodiment, the template strand includes a signal peptide, e.g., a eukaryotic or prokaryotic signal peptide.

In another embodiment, the template includes a nucleic acid sequence that encodes an enzyme or an inactivated enzyme, (e.g., as the sequence to be varied)

In one embodiment, the diversified nucleic acids are homologous (e.g., at least 30% homologous, more preferably at least about 40%, 50%, 60%, 70%, 80%, 90%, or more homologous) to one of the plurality of template nucleic acid strands. Preferably the diversified nucleic acids are homologous to each template nucleic acid strand of the plurality. In another embodiment, the diversified nucleic acids are homologous (e.g., at least 30% homologous, more preferably at least about 40%, 50%, 60%, 70%, or more homologous) to a reference domain, and each of the template nucleic acids is homologous to the reference domain.

In one embodiment, the annealed oligonucleotide is both extended and ligated. In another embodiment, the annealed oligonucleotide is extended. The extending and/or ligating can occur at least partially in a cell. Preferably, the extending and/or ligating occurs in vitro. The extending can be effected by a DNA polymerase or an RNA polymerase. Examples of DNA polymerases include E. coli polymerase I, T4 DNA polymerase, and reverse transcriptase (an RNA-dependent DNA polymerase). In a preferred embodiment, the DNA polymerase is a non-strand displacing DNA polymerase (e.g., T4 or T7 DNA polymerase). In another preferred embodiment, the DNA polymerase is a thermostable DNA polymerase. Another preferred DNA polymerase is the Klenow fragment of E. coli polymerase I or any DNA polymerase that lacks a 3′ to 5′ exonuclease activity.

In one embodiment, the method includes separating the diversified strand from the template strand. In another embodiment, the method includes separating diversified strand-template strand heteroduplexes from homoduplexes, e.g., using a mismatch binding protein. In another embodiment, the method can further include one or more of: amplifying the diversified strand, selectively disabling the template strand, and isolating the diversified strand.

The method can further include ligating the extended, hybridized diverse oligonucleotides. The method can include optionally introducing the diversified strand, a replicate, or complement thereof into cells, and/or optionally, translating the diversified strand, a replicate, or complement thereof.

In a preferred embodiment, the method further includes synthesizing a polypeptide encoded by the diversified strand or its complement. The translating can be in vitro or in vivo (i.e., in a host cell, e.g., a cultured cell or a transgenic cell that is part of an animal or plant). The host cell can be a prokaryotic cell (e.g., a bacterial cell) or is eukaryotic cell (e.g., a fungal cell, such as yeast, or a mammalian cell).

In one embodiment, the polypeptide is attached to the host cell surface (e.g., a yeast or mammalian cell surface, e.g., by means of a transmembrane protein or domain thereof or a peripheral membrane protein) or a virus surface, e.g., a filamentous phage coat protein or fragment thereof. The attachment can be direct or indirect (e.g., bridged), and can be covalent or non-covalent. In another embodiment, the polypeptide is attached to a solid support, e.g., a bead, particle, three-dimensional matrix, or planar array.

The method can further include constructing a library that includes the diversified strands, e.g., by introducing the diversified strand into a host cells with other diversified strands.

The method can further include screening the diversified strands or the complements thereof, e.g., using a method described herein. Exemplary methods include a display library, a polypeptide array, an in vitro assay, or an in vivo assay.

In still another aspect, the invention features a method that includes: a) providing i) a template nucleic acid strand, and ii) a plurality of diverse oligonucleotides, wherein the template nucleic acid strand or complement thereof encodes an immunoglobulin variable domain and each diverse oligonucleotide of the plurality encodes a sequence that includes at least a portion of a CDR; b) contacting the plurality of diverse oligonucleotides and the template nucleic acid strand in a mixture; c) subjecting the mixture to conditions such that only a subset of the plurality of diverse oligonucleotides can anneal to the template nucleic acid strand; and d) extending and/or ligating an annealed oligonucleotide of the subset to form a diversified strand that is partially complementary to the template nucleic acid strand.

In a preferred embodiment, each of the diverse oligonucleotides encodes a sequence that includes a CDR (e.g., a whole CDR). In another preferred embodiment, each of the diverse oligonucleotides encodes a sequence that flanks a CDR (e.g., part of a framework region). In still another embodiment, each of the diverse oligonucleotides encodes a sequence that does not include a CDR flanking region (e.g., part of a framework region). In a preferred embodiment, each diverse oligonucleotide includes 3′ and 5′ terminal regions that anneal to a sequence that flanks a sequence encoding a CDR (or its complement), e.g., a sequence that encodes a framework region (or its complement), e.g., at least one, two, three, four, or five nucleotides thereof. The terminal regions are preferably less varied than the sequence between the terminal regions among the diverse oligonucleotides. The CDR can be a heavy chain CDR (e.g., heavy chain CDR1, CDR2, and CDR3) or a light chain CDR (e.g., light chain CDR1, CDR2, and CDR3). In a preferred embodiment the diverse oligonucleotides preferably do not include the entire sequence of the framework regions which flank the CDR, e.g., contain less than 2, 5, 8, 10, or 15 of the amino acids of each of the flanking framework regions.

In a preferred embodiment, the immunoglobulin variable domain comprises a VH or VL domain. The sequence can further include an immunoglobulin constant domain, e.g., a CH1 or CL. With respect to a VH domain, the template can further include a sequence encoding a CH2 and CH3 domain. The VH or VL domain can include a synthetic CDR or a germline CDR (e.g., a human CDR). Further, the VH or VL domain can include a framework region, e.g., a human framework region. For example, the polypeptide can include a VH and CH1 domain or a VL and CL domain. The polypeptide can include both a VH and VL domain, e.g., as a single-chain Fv domain (ScFv). The polypeptide can be such that the VH and VL domains form, e.g., Fab fragments, F(ab′)₂, Fv fragments, and single-chain Fv fragments. The polypeptides include an antigen binding site, e.g., a functional antigen binding site. In a preferred embodiment the template includes at least one, and preferably two or three CDRs, and all or part of at least one framework region. For example, it can include at least one CDR, e.g., a CDR1, and all or part of the framework regions which flank CDR1.

In another preferred embodiment, the template nucleic acid encodes a second polypeptide. The first and second polypeptide can form a complex, e.g., the first and second polypeptide can be non-covalently bound or covalently bound, e.g., by one or more disulfides. For example, the complex can include a Fab.

In one embodiment, the providing of diverse oligonucleotides includes: providing a plurality of diverse nucleic acids; annealing at least a first pair of cleavage-directing oligonucleotides to a given strand of each diverse nucleic acid of the plurality to form cleavable regions for each given strand; and cleaving the cleavable regions of each given strand to yield at least the plurality of diverse oligonucleotides from the given strands, each diverse oligonucleotide being unique in the plurality of diverse oligonucleotides. In a preferred embodiment, the diverse nucleic acids are obtained from a cDNA pool from B cells, e.g., human B cells, e.g., from a subject afflicted with peripheral blood syndrome, vasculitis, an autoimmune disorder, or a neoplastic disorder. For example, the method can further include reverse transcribing cDNA from mRNA isolated from B cells.

In one embodiment, the cleavage-directing oligonucleotide includes a stem-loop structure, e.g., a structure that includes a recognition site for a Type IIS restriction enzyme. The cleaving is effected by the Type IIS restriction enzyme. In another embodiment, the cleaving is effected by a Type II restriction enzyme, e.g., an enzyme hat recognizes a site of six basepairs, or less than six basepairs, e.g., five or four basepairs. In a preferred embodiment, the cleaving occurs at a temperature greater than 40° C.

In one embodiment, the cleavage-directing oligonucleotide forms a heteroduplex with the diverse nucleic acid and the cleavable region is fully complementary to the diverse nucleic acid within the heteroduplex. In a preferred embodiment, at least two cleavage-directing oligonucleotides are annealed to each of the diverse nucleic acids, e.g., one directs the cleavage of a 5′ terminus of a diverse oligonucleotide and the other directs the cleavage of a 3′ terminus of the diverse oligonucleotide. In a preferred embodiment, at least three pairs of cleavage-directing oligonucleotides are annealed. For example, the pairs can release at least one, two, or three diverse oligonucleotides. In one embodiment, the released diverse oligonucleotides encode one or more of: CDR1, CDR2, and CDR3 of an immunoglobulin variable domain. The diverse oligonucleotides can be released sequentially or concurrently.

In one embodiment, the diverse oligonucleotides include at least 10³, 10⁴, 10⁵, 10⁶, 10⁸, 10⁹, or 10¹⁰ different oligonucleotides. In one embodiment, each diverse oligonucleotide is less than 200, 120, 80, 70, 65, 60, 55, 50, 45, 40, or 35 nucleotides in length. The diverse oligonucleotides can be at least about 20, 25, 30, 35, 40, 45, 50, or 60 nucleotides in length. Each diverse oligonucleotide can be at least 40%, 50%, 60%, 70%, 80%, 90%, 95%, or 98% identical to at least another diverse oligonucleotide. For example, a diverse oligonucleotide can have 1, 2, 3, or at least 4 mismatches with respect to another diverse oligonucleotide.

In a preferred embodiment, each of the diverse oligonucleotides is of equal length as the others or are within 30, 20, 15, or 10% of the average length of the diverse oligonucleotides. In one embodiment, the diverse oligonucleotides of the plurality all have a length within 8, 6, 4, 3, 2, or 1 nucleotide of each other. Each of the diverse oligonucleotides can include 3′ and/or 5′ terminal regions of at least 6 nucleotides in length that are identical (or at least 70% identical) to corresponding terminal regions of each of the other diverse oligonucleotides. The terminal regions can be between 6 and 20 nucleotides in length, e.g., between 6 and 15, or 10 and 18 nucleotides in length. In a preferred embodiment, the terminal regions are exactly complementary to a corresponding site on the template nucleic acid. In a preferred embodiment, each of the diverse oligonucleotides includes a sequence corresponding to (e.g., partially complementary to) a common region of the template (e.g., of at least 5 or 10 nucleotides). Each diverse oligonucleotide can include a naturally occurring sequence or a synthetic sequence. The diverse oligonucleotides can be constructed by chemical synthesis. In another embodiment, the diverse oligonucleotides are constructed by cleavage of a diverse nucleic acid strand.

In another preferred embodiment, each diverse-oligonucleotide encodes an enzyme active site residue, e.g., a residue that is within 2 Angstroms of a bound substrate or cofactor.

In one embodiment, the diverse nucleic acids include at least 10³, 10⁴, 10⁵, 10⁶, 10⁸, 10⁹, or 10¹⁰ different nucleic acids. The diverse nucleic acids can be, e.g., mRNA, cDNA, or genomic nucleic acids. Each diverse nucleic acid can be fixed to a solid support. In a preferred embodiment, the diverse nucleic acids are obtained from a mammalian cell, e.g., a hematopoietic cell such as a B or T cell. In another preferred embodiment, the mammalian cell is obtained from a subject having an immune disorder. The diverse nucleic acids can be obtained from a mammalian cell cultured in vitro. The cell can also be stimulated to undergo somatic mutagenesis of immunoglobulin genes, class switching of immunoglobulin genes, or proliferation.

The template nucleic acid can encode a polypeptide of at least 10, 20, 50, 100, or 200 amino acids.

In one embodiment, the polypeptide has a binding activity or is preselected for a binding activity. In another embodiment, the polypeptide has an enzymatic activity or is preselected for an enzymatic activity (e.g., the polypeptide is a catalytic antibody). The polypeptide can be naturally occurring or synthetic, e.g., partially synthetic, e.g., a synthetic variant of a naturally occurring polypeptide. The preselecting can include identifying the polypeptide from a display library on the basis of the binding activity.

The combining can include annealing at least some of the diverse oligonucleotides to the template nucleic acid strand. In one embodiment, the annealed oligonucleotides include diverse oligonucleotides of the subset and diverse oligonucleotides not of the subset to the template nucleic acid strand. Subsequent washing of the template nucleic acid strand dissociates the hybridized diverse oligonucleotides not of the subset. In another embodiment, the annealed diverse oligonucleotides are exclusively from the subset.

In one embodiment the conditions for the contacting include a temperature greater than 40° C. In another embodiment, the conditions include a temperature within 10 or 5° C. of a Tₘ, or a temperature greater than Tₘ-10°, Tₘ-5°, or Tₘ, wherein the Tₘ is the Tₘ of a segment of the template nucleic acid strand for its exact complement, and the segment is the region to which the diverse oligonucleotides hybridize. In one embodiment, the selected solution conditions are approximately a condition listed in Table 1. The hybridization conditions can include formamide or urea. The hybridization conditions can be selected so as to result in a preferred level of variation in the product, e.g., wherein the resulting molecules are at least 70, 80, 85, 90, 95, 97, or 98% homologous to the template. In some embodiments the level of homology is with regard to the entire length of the template, while in others it is with regard to the regions which correspond to diverse oligonucleotides.

In one embodiment, the template nucleic acid strand is limiting, and, for example, each diversity oligonucleotide of the population competes for the template nucleic acid strand under equilibrium binding conditions, e.g., conditions selected to favor competitive binding. In another embodiment, the template nucleic acid strand is not limiting. Exemplary molar ratios for the template nucleic acid strand to the diversity oligonucleotides include between 100:1 and 1:100; 10:1 and 1:10; 5:1 and 1:5; 10:1 and 1:1; 1:1 and 1:10.

In one embodiment, the subjecting includes separating at least some of the subset of diverse oligonucleotides that can anneal to the template nucleic acid strand from the remaining diverse oligonucleotides of the plurality. The separating can include washing the template nucleic acid strand. The template nucleic acid can be attached to a solid support. For example, the template nucleic acid strand can be immobilized on a solid support, e.g., by a covalent or non-covalent linkage. The washing conditions can be more stringent than conditions for the contacting. In another embodiment, the separating includes a size separation, e.g., using a membrane porous to unannealed diverse oligonucleotides but not annealed diverse oligonucleotides, a gel exclusion method, a sedimentation method, or an electrophoretic method.

In a preferred embodiment, a plurality of template nucleic acid strands are provided. The template nucleic acid strands of the plurality can differ from one another. For example, the template nucleic acid strands can be at least 50% (e.g., at least 60%, 70%, or 80%) identical to each other. The template nucleic acid strands can encode polypeptides that share the same scaffold domain. In a preferred embodiment, each template nucleic acid strand of the plurality encodes a polypeptide that has an activity or is preselected for an activity.

For example, the providing of one or more template nucleic acids includes: (1) providing a display library, each member of which includes a nucleic acid that encodes a polypeptide and the encoded polypeptide; (2) identifying members of the display library for which the encoded polypeptide has at least a threshold activity; and (3) providing (e.g., isolating) template nucleic acid replicates for at least one of the identified members of the display library.

In one embodiment, each template strand or template strand complement encodes a polypeptide domain that, preferably, has at least a threshold activity. The method can further include screening the polypeptide encoded by the diversified strand complement (or a complement thereof), e.g., for an improved level of activity that exceeds the threshold activity. The threshold activity can be less than about 50, 10, 1, 0.1, or 0.01% of the improved level of activity. In a preferred embodiment, the one or more template nucleic acid strands is a plurality of template nucleic acid strands. In one embodiment, each template nucleic acid strand of the plurality of template nucleic acid strands is the same. In another embodiment, the plurality of template nucleic acid strands includes at least 2, 4, 8, 12, 30, 100, or 150 different template nucleic acid strands. In a preferred embodiment, the plurality of template nucleic acid strands includes different strands such that each or its complement encodes a polypeptide that includes a domain with at least a threshold activity of interest. For example, the strands of the plurality can include strands, each encoding a different polypeptide that is homologous (e.g., at least 40, 50, 60, 70, 80, 90, 95%) to the other encoded polypeptides, and/or has at least a threshold activity, e.g., a threshold measure of the same activity as the other polypeptides.

In another embodiment, the sequence of the template nucleic acid strand is not known at the time of the annealing. For example, the complete sequence of the template nucleic acid strand may be undetermined. More particularly, the sequence of the template nucleic acid strand in a region to which a diversity oligonucleotide can anneal is not known at the time of the annealing. An example of such a region is a region that encodes a CDR of an immunoglobulin variable domain.

In a preferred embodiment, the template nucleic acid(s) comprise DNA. In another embodiment, they comprise RNA.

The template strand can be linear or circular. In one embodiment, the template strand is immobilized on a solid support, e.g., using a covalent or non-covalent linkage. The template strand can include uracil at at least some nucleotides. The template strand can further include a unique restriction enzyme site, one or more selectable markers, e.g., one functional selectable marker and one marker that includes a lesion, one or more bacteriophage genes, e.g., a gene encoding a major or minor coat protein, e.g., filamentous phage gene III. Each template nucleic acid can be tagged or fixed to a solid support.

In another embodiment, the template nucleic strand includes a sequence encoding a transcription factor functional domain (e.g., for a two-hybrid assay), a cytotoxin, a label (e.g., green fluorescent protein or luciferase).

In one embodiment, the template strand comprises a promoter, e.g., a prokaryotic promoter, e.g., a bacteriophage promoter such as the T7, T3, or SP6 promoter. In another embodiment, the template strand includes a signal peptide, e.g., a eukaryotic or prokaryotic signal peptide.

In another embodiment, the template includes a nucleic acid sequence that encodes an enzyme or an inactivated enzyme, (e.g., as the sequence to be varied)

In one embodiment, the diversified nucleic acids are homologous (e.g., at least 30% homologous, more preferably at least about 40%, 50%, 60%, 70%, 80%, 90%, or more homologous) to one of the plurality of template nucleic acid strands. Preferably the diversified nucleic acids are homologous to each template nucleic acid strand of the plurality. In another embodiment, the diversified nucleic acids are homologous (e.g., at least 30% homologous, more preferably at least about 40%, 50%, 60%, 70%, or more homologous) to a reference domain, and each of the template nucleic acids is homologous to the reference domain.

In one embodiment, the annealed oligonucleotide is both extended and ligated. In another embodiment, the annealed oligonucleotide is extended. The extending and/or ligating can occur at least partially in a cell. Preferably, the extending and/or ligating occurs in vitro. The extending can be effected by a DNA polymerase or an RNA polymerase. Examples of DNA polymerases include E. coli polymerase I, T4 DNA polymerase, and reverse transcriptase (an RNA-dependent DNA polymerase). In a preferred embodiment, the DNA polymerase is a non-strand displacing DNA polymerase (e.g., T4 or T7 DNA polymerase). In another preferred embodiment, the DNA polymerase is a thermostable DNA polymerase. Another preferred DNA polymerase is the Klenow fragment of E. coli polymerase I or any DNA polymerase that lacks a 3′ to 5′ exonuclease activity.

In one embodiment, the method includes separating the diversified strand from the template strand. In another embodiment, the method includes separating diversified strand-template strand heteroduplexes from homoduplexes, e.g., using a mismatch binding protein. In another embodiment, the method can further include one or more of: amplifying the diversified strand, selectively disabling the template strand, and isolating the diversified strand.

The method can further include ligating the extended, hybridized diverse oligonucleotides. The method can include optionally introducing the diversified strand, a replicate, or complement thereof into cells, and/or optionally, translating the diversified strand, a replicate, or complement thereof.

In a preferred embodiment, the method further includes synthesizing a polypeptide encoded by the diversified strand or its complement. The translating can be in vitro or in vivo (i.e., in a host cell, e.g., a cultured cell or a transgenic cell that is part of an animal or plant). The host cell can be a prokaryotic cell (e.g., a bacterial cell) or is eukaryotic cell (e.g., a fungal cell, such as yeast, or a mammalian cell).

In one embodiment, the polypeptide is attached to the host cell surface (e.g., a yeast or mammalian cell surface, e.g., by means of a transmembrane protein or domain thereof or a peripheral membrane protein) or a virus surface, e.g., a filamentous phage coat protein or fragment thereof. The attachment can be direct or indirect (e.g., bridged), and can be covalent or non-covalent. In another embodiment, the polypeptide is attached to a solid support, e.g., a bead, particle, three-dimensional matrix, or planar array.

The method can further include constructing a library that includes the diversified strands, e.g., by introducing the diversified strand into a host cells with other diversified strands.

The method can further include screening the diversified strands or the complements thereof, e.g., using a method described herein. Exemplary methods include a display library, a polypeptide array, an in vitro assay, or an in vivo assay.

In another aspect, the invention features a method that includes: a) providing i) a template nucleic acid strand and ii) a plurality of diverse oligonucleotides, wherein each diverse oligonucleotide of the plurality (1) is of equal length as the other diverse oligonucleotides or within 10% (or within 30, 20, 10, 5%) of the average of all the diverse oligonucleotide lengths, and/or (2) includes 3′ and 5′ terminal regions of at least 6 nucleotides in length, the terminal regions being substantially identical (or at least 70% identical) to the corresponding terminal regions of each of the other diverse oligonucleotides; b) contacting the plurality of diverse oligonucleotides and the template nucleic acid strand in a mixture; c) subjecting the mixture to conditions such that only a subset of the plurality of diverse oligonucleotides can anneal to the template nucleic acid strand; and d) extending and/or ligating an annealed oligonucleotide of the subset to form a diversified strand that is partially complementary to the template nucleic acid strand. In one embodiment, the providing of diverse oligonucleotides includes: providing a plurality of diverse nucleic acids; annealing at least a first pair of cleavage-directing oligonucleotides to a given strand of each diverse nucleic acid of the plurality to form cleavable regions for each given strand; and cleaving the cleavable regions of each given strand to yield at least the plurality of diverse oligonucleotides from the given strands, each diverse oligonucleotide being unique in the plurality of diverse oligonucleotides. In a preferred embodiment, the diverse nucleic acids are obtained from a cDNA pool from B cells, e.g., human B cells, e.g., from a subject afflicted with peripheral blood syndrome, vasculitis, an autoimmune disorder, or a neoplastic disorder. For example, the method can further include reverse transcribing cDNA from mRNA isolated from B cells.

In one embodiment, the cleavage-directing oligonucleotide includes a stem-loop structure, e.g., a structure that includes a recognition site for a Type IIS restriction enzyme. The cleaving is effected by the Type IIS restriction enzyme. In another embodiment, the cleaving is effected by a Type II restriction enzyme, e.g., an enzyme hat recognizes a site of six basepairs, or less than six basepairs, e.g., five or four basepairs. In a preferred embodiment, the cleaving occurs at a temperature greater than 40° C.

In one embodiment, the cleavage-directing oligonucleotide forms a heteroduplex with the diverse nucleic acid and the cleavable region is fully complementary to the diverse nucleic acid within the heteroduplex. In a preferred embodiment, at least two cleavage-directing oligonucleotides are annealed to each of the diverse nucleic acids, e.g., one directs the cleavage of a 5′ terminus of a diverse oligonucleotide and the other directs the cleavage of a 3′ terminus of the diverse oligonucleotide. In a preferred embodiment, at least three pairs of cleavage-directing oligonucleotides are annealed. For example, the pairs can release at least one, two, or three diverse oligonucleotides. In one embodiment, the released diverse oligonucleotides encode one or more of: CDR1, CDR2, and CDR3 of an immunoglobulin variable domain. The diverse oligonucleotides can be released sequentially or concurrently.

In one embodiment, the diverse oligonucleotides include at least 10³, 10⁴, 10⁵, 10⁶, 10⁸, 10⁹, or 10¹⁰ different oligonucleotides. In one embodiment, each diverse oligonucleotide is less than 200, 120, 80, 70, 65, 60, 55, 50, 45, 40, or 35 nucleotides in length. The diverse oligonucleotides can be at least about 20, 25, 30, 35, 40, 45, 50, or 60 nucleotides in length. Each diverse oligonucleotide can be at least 40%, 50%, 60%, 70%, 80%, 90%, 95%, or 98% identical to at least another diverse oligonucleotide. For example, a diverse oligonucleotide can have 1, 2, 3, or at least 4 mismatches with respect to another diverse oligonucleotide.

In a preferred embodiment, each of the diverse oligonucleotides is of equal length as the other diverse oligonucleotides. In one embodiment, the diverse oligonucleotides of the plurality all have a length within 8, 6, 4, 3, 2, or 1 nucleotide of each other. In a preferred embodiment, the terminal regions are exactly complementary to a corresponding site on the template nucleic acid. In a preferred embodiment, each of the diverse oligonucleotides includes a sequence corresponding to (e.g., partially complementary to) a common region of the template (e.g., of at least 5 or 10 nucleotides). Each diverse oligonucleotide can include a naturally occurring sequence or a synthetic sequence. The diverse oligonucleotides can be constructed by chemical synthesis. In another embodiment, the diverse oligonucleotides are constructed by cleavage of a diverse nucleic acid strand.

In a preferred embodiment, each diverse oligonucleotide encodes a CDR or fragment thereof, e.g., a fragment including at least 5 amino acids. In a much preferred embodiment, the diverse oligonucleotides further include 3′ and/or 5′ terminal regions that anneal to a sequence that flanks a sequence encoding a CDR (or its complement), e.g., a sequence that encodes a framework region (or its complement), e.g., at least one, two, three, four, or five nucleotides thereof. The terminal regions are preferably less varied than the sequence between the terminal regions among the diverse oligonucleotides. The CDR can be a heavy chain CDR (e.g., heavy chain CDR1, CDR2, and CDR3) or a light chain CDR (e.g., light chain CDR1, CDR2, and CDR3). In a preferred embodiment the diverse oligonucleotides preferably do not include the entire sequence of the framework regions which flank the CDR, e.g., contain less than 2, 5, 8, 10, or 15 of the amino acids of each of the flanking framework regions.

In another preferred embodiment, each diverse oligonucleotide encodes an enzyme active site residue, e.g., a residue that is within 2 Angstroms of a bound substrate or cofactor.

In one embodiment, the diverse nucleic acids include at least 10³, 10⁴, 10⁵, 10⁶, 10⁸, 10⁹, or 10¹⁰ different nucleic acids. The diverse nucleic acids can be, e.g., mRNA, cDNA, or genomic nucleic acids. Each diverse nucleic acid can be fixed to a solid support. In a preferred embodiment, the diverse nucleic acids are obtained from a mammalian cell, e.g., a hematopoietic cell such as a B or T cell. In another preferred embodiment, the mammalian cell is obtained from a subject having an immune disorder. The diverse nucleic acids can be obtained from a mammalian cell cultured in vitro. The cell can also be stimulated to undergo somatic mutagenesis of immunoglobulin genes, class switching of immunoglobulin genes, or proliferation.

The template nucleic acid can encode a polypeptide of at least 10, 20, 50, 100, or 200 amino acids. For example, the polypeptide can include a domain of a cell surface protein, an enzyme, a T cell receptor, an MHC protein, a protease inhibitor, a scaffold domain, or a transcription factor. In one embodiment, the polypeptide does not include an immunoglobulin domain, i.e., the polypeptide is not an antibody.

In one embodiment, the polypeptide has a binding activity or is preselected for a binding activity. In another embodiment, the polypeptide has an enzymatic activity or is preselected for an enzymatic activity. The polypeptide can be naturally occurring or synthetic, e.g., partially synthetic, e.g., a synthetic variant of a naturally occurring polypeptide. The preselecting can include identifying the polypeptide from a display library on the basis of the binding activity.

In a preferred embodiment, the polypeptide includes an immunoglobulin domain, e.g., a variable domain, e.g., a VH or VL domain. The sequence can further include an immunoglobulin constant domain, e.g., a CH1 or CL. With respect to a VH domain, the template can further include a sequence encoding a CH2 and CH3 domain. The VH or VL domain can include a synthetic CDR or a germline CDR (e.g., a human CDR). Further, the VH or VL domain can include a framework region, e.g., a human framework region. For example, the polypeptide can include a VH and CH1 domain or a VL and CL domain. The polypeptide can include both a VH and VL domain, e.g., as a single-chain Fv domain (ScFv). The polypeptide can be such that the VH and VL domains form, e.g., Fab fragments, F(ab′)₂, Fv fragments, and single-chain Fv fragments. The polypeptides include an antigen binding site, e.g., a functional antigen binding site. In a preferred embodiment the template includes at least one, and preferably two or three CDRs, and all or part of at least one framework region. For example, it can include at least one CDR, e.g., a CDR1, and all or part of the framework regions which flank CDR1.

In another preferred embodiment, the template nucleic acid encodes a second polypeptide. The first and second polypeptide can form a complex, e.g., the first and second polypeptide can be non-covalently bound or covalently bound, e.g., by one or more disulfides. For example, the complex can include a Fab.

The combining can include annealing at least some of the diverse oligonucleotides to the template nucleic acid strand. In one embodiment, the annealed oligonucleotides include diverse oligonucleotides of the subset and diverse oligonucleotides not of the subset to the template nucleic acid strand. Subsequent washing of the template nucleic acid strand dissociates the hybridized diverse oligonucleotides not of the subset. In another embodiment, the annealed diverse oligonucleotides are exclusively from the subset.

In one embodiment the conditions for the contacting include a temperature greater than 40° C. In another embodiment, the conditions include a temperature within 10 or 5° C. of a Tₘ, or a temperature greater than Tₘ-10^{°, T}ₘ-5°, or Tₘ, wherein the Tₘ is the Tₘ of a segment of the template nucleic acid strand for its exact complement, and the segment is the region to which the diverse oligonucleotides hybridize. In one embodiment, the selected solution conditions are approximately a condition listed in Table 1. The hybridization conditions can include formamide or urea. The hybridization conditions can be selected so as to result in a preferred level of variation in the product, e.g., wherein the resulting molecules are at least 70, 80, 85, 90, 95, 97, or 98% homologous to the template. In some embodiments the level of homology is with regard to the entire length of the template, while in others it is with regard to the regions that correspond to diverse oligonucleotides.

In one embodiment, the template nucleic acid strand is limiting, and, for example, each diversity oligonucleotide of the population competes for the template nucleic acid strand under equilibrium binding conditions, e.g., conditions selected to favor competitive binding. In another embodiment, the template nucleic acid strand is not limiting. Exemplary molar ratios for the template nucleic acid strand to the diversity oligonucleotides include between 100:1 and 1:100; 10:1 and 1:10; 5:1 and 1:5; 10:1 and 1:1; 1:1 and 1:10.

In one embodiment, the subjecting includes separating at least some of the subset of diverse oligonucleotides that can anneal to the template nucleic acid strand from the remaining diverse oligonucleotides of the plurality. The separating can include washing the template nucleic acid strand. The template nucleic acid can be attached to a solid support. For example, the template nucleic acid strand can be immobilized on a solid support, e.g., by a covalent or non-covalent linkage. The washing conditions can be more stringent than conditions for the contacting. In another embodiment, the separating includes a size separation, e.g., using a membrane porous to unannealed diverse oligonucleotides but not annealed diverse oligonucleotides, a gel exclusion method, a sedimentation method, or an electrophoretic method.

In a preferred embodiment, a plurality of template nucleic acid strands are provided. The template nucleic acid strands of the plurality can differ from one another. For example, the template nucleic acid strands can be at least 50% (e.g., at least 60%, 70%, or 80%) identical to each other. The template nucleic acid strands can encode polypeptides that share the same scaffold domain. In a preferred embodiment, each template nucleic acid strand of the plurality encodes a polypeptide that has an activity or is preselected for an activity.

For example, the providing of one or more template nucleic acids includes: (1) providing a display library, each member of which includes a nucleic acid that encodes a polypeptide and the encoded polypeptide; (2) identifying members of the display library for which the encoded polypeptide has at least a threshold activity; and (3) providing (e.g., isolating) template nucleic acid replicates for at least one of the identified members of the display library.

In one embodiment, each template strand or template strand complement encodes a polypeptide domain that, preferably, has at least a threshold activity. The method can further include screening the polypeptide encoded by the diversified strand complement (or a complement thereof), e.g., for an improved level of activity that exceeds the threshold activity. The threshold activity can be less than about 50, 10, 1, 0.1, or 0.01% of the improved level of activity. In a preferred embodiment, the one or more template nucleic acid strands is a plurality of template nucleic acid strands. In one embodiment, each template nucleic acid strand of the plurality of template nucleic acid strands is the same. In another embodiment, the plurality of template nucleic acid strands includes at least 2, 4, 8, 12, 30, 100, or 150 different template nucleic acid strands. In a preferred embodiment, the plurality of template nucleic acid strands includes different strands such that each or its complement encodes a polypeptide that includes a domain with at least a threshold activity of interest. For example, the strands of the plurality can include strands, each encoding a different polypeptide that is homologous (e.g., at least 40, 50, 60, 70, 80, 90, 95%) to the other encoded polypeptides, and/or has at least a threshold activity, e.g., a threshold measure of the same activity as the other polypeptides.

In another embodiment, the sequence of the template nucleic acid strand is not known at the time of the annealing. For example, the complete sequence of the template nucleic acid strand may be undetermined. More particularly, the sequence of the template nucleic acid strand in a region to which a diversity oligonucleotide can anneal is not known at the time of the annealing. An example of such a region is a region that encodes a CDR of an immunoglobulin variable domain.

In a preferred embodiment, the template nucleic acid(s) comprise DNA. In another embodiment, they comprise RNA.

The template strand can be linear or circular. In one embodiment, the template strand is immobilized on a solid support, e.g., using a covalent or non-covalent linkage. The template strand can include uracil at at least some nucleotides. The template strand can further include a unique restriction enzyme site, one or more selectable markers, e.g., one functional selectable marker and one marker that includes a lesion, one or more bacteriophage genes, e.g., a gene encoding a major or minor coat protein, e.g., filamentous phage gene III. Each template nucleic acid can be tagged or fixed to a solid support.

In another embodiment, the template nucleic strand includes a sequence encoding a transcription factor functional domain (e.g., for a two-hybrid assay), a cytotoxin, a label (e.g., green fluorescent protein or luciferase).

In one embodiment, the template strand comprises a promoter, e.g., a prokaryotic promoter, e.g., a bacteriophage promoter such as the T7, T3, or SP6 promoter. In another embodiment, the template strand includes a signal peptide, e.g., a eukaryotic or prokaryotic signal peptide.

In another embodiment, the template includes a nucleic acid sequence that encodes an enzyme or an inactivated enzyme, (e.g., as the sequence to be varied)

In one embodiment, the diversified nucleic acids are homologous (e.g., at least 30% homologous, more preferably at least about 40%, 50%, 60%, 70%, 80%, 90%, or more homologous) to one of the plurality of template nucleic acid strands. Preferably the diversified nucleic acids are homologous to each template nucleic acid strand of the plurality. In another embodiment, the diversified nucleic acids are homologous (e.g., at least 30% homologous, more preferably at least about 40%, 50%, 60%, 70%, or more homologous) to a reference domain, and each of the template nucleic acids is homologous to the reference domain.

In one embodiment, the annealed oligonucleotide is both extended and ligated. In another embodiment, the annealed oligonucleotide is extended. The extending and/or ligating can occur at least partially in a cell. Preferably, the extending and/or ligating occurs in vitro. The extending can be effected by a DNA polymerase or an RNA polymerase. Examples of DNA polymerases include E. coli polymerase I, T4 DNA polymerase, and reverse transcriptase (an RNA-dependent DNA polymerase). In a preferred embodiment, the DNA polymerase is a non-strand displacing DNA polymerase (e.g., T4 or T7 DNA polymerase). In another preferred embodiment, the DNA polymerase is a thermostable DNA polymerase. Another preferred DNA polymerase is the Klenow fragment of E. coli polymerase I or any DNA polymerase that lacks a 3′ to 5′ exonuclease activity.

In one embodiment, the method includes separating the diversified strand from the template strand. In another embodiment, the method includes separating diversified strand-template strand heteroduplexes from homoduplexes, e.g., using a mismatch binding protein. In another embodiment, the method can further include one or more of: amplifying the diversified strand, selectively disabling the template strand, and isolating the diversified strand.

The method can further include ligating the extended, hybridized diverse oligonucleotides. The method can include optionally introducing the diversified strand, a replicate, or complement thereof into cells, and/or optionally, translating the diversified strand, a replicate, or complement thereof.

In a preferred embodiment, the method further includes synthesizing a polypeptide encoded by the diversified strand or its complement. The translating can be in vitro or in vivo (i.e., in a host cell, e.g., a cultured cell or a transgenic cell that is part of an animal or plant). The host cell can be a prokaryotic cell (e.g., a bacterial cell) or is eukaryotic cell (e.g., a fungal cell, such as yeast, or a mammalian cell).

In one embodiment, the polypeptide is attached to the host cell surface (e.g., a yeast or mammalian cell surface, e.g., by means of a transmembrane protein or domain thereof or a peripheral membrane protein) or a virus surface, e.g., a filamentous phage coat protein or fragment thereof. The attachment can be direct or indirect (e.g., bridged), and can be covalent or non-covalent. In another embodiment, the polypeptide is attached to a solid support, e.g., a bead, particle, three-dimensional matrix, or planar array.

The method can further include constructing a library that includes the diversified strands, e.g., by introducing the diversified strand into a host cells with other diversified strands.

The method can further include screening the diversified strands or the complements thereof, e.g., using a method described herein. Exemplary methods include a display library, a polypeptide array, an in vitro assay, or an in vivo assay.

In another aspect, the invention features a method that includes: a) providing a display library and a plurality of diverse oligonucleotides; b) identifying members of the display library which display polypeptides that have at least a threshold degree of a given activity; c) providing (e.g., isolating) template nucleic acid replicates for at least one of the identified members of the display library; d) combining the plurality of diverse oligonucleotides and the template nucleic acid replicates in a mixture; e) subjecting the mixture to conditions such that only a subset of the plurality of diverse oligonucleotides can anneal to the template nucleic acid replicates; and f) extending and/or ligating an annealed oligonucleotide of the subset to form a diversified strand that is partially complementary to the template nucleic acid strand.

The display library can be a phage display library or a cell display library, e.g., a eukaryotic cell display library, e.g., a yeast display library.

In one embodiment, the replicates of each template nucleic acid are combined with the diverse oligonucleotides in a separate container from the replicates of the other template nucleic acids. In another embodiment, they are combined in the same container.

In one embodiment, the providing of diverse oligonucleotides includes: providing a plurality of diverse nucleic acids; annealing at least a first pair of cleavage-directing oligonucleotides to a given strand of each diverse nucleic acid of the plurality to form cleavable regions for each given strand; and cleaving the cleavable regions of each given strand to yield at least the plurality of diverse oligonucleotides from the given strands, each diverse oligonucleotide being unique in the plurality of diverse oligonucleotides. In a preferred embodiment, the diverse nucleic acids are obtained from a cDNA pool from B cells, e.g., human B cells, e.g., from a subject afflicted with peripheral blood syndrome, vasculitis, an autoimmune disorder, or a neoplastic disorder. For example, the method can further include reverse transcribing cDNA from mRNA isolated from B cells.

In one embodiment, the cleavage-directing oligonucleotide includes a stem-loop structure, e.g., a structure that includes a recognition site for a Type IIS restriction enzyme. The cleaving is effected by the Type IIS restriction enzyme. In another embodiment, the cleaving is effected by a Type II restriction enzyme, e.g., an enzyme hat recognizes a site of six basepairs, or less than six basepairs, e.g., five or four basepairs. In a preferred embodiment, the cleaving occurs at a temperature greater than 40° C.

In one embodiment, the cleavage-directing oligonucleotide forms a heteroduplex with the diverse nucleic acid and the cleavable region is fully complementary to the diverse nucleic acid within the heteroduplex. In a preferred embodiment, at least two cleavage-directing oligonucleotides are annealed to each of the diverse nucleic acids, e.g., one directs the cleavage of a 5′ terminus of a diverse oligonucleotide and the other directs the cleavage of a 3′ terminus of the diverse oligonucleotide. In a preferred embodiment, at least three pairs of cleavage-directing oligonucleotides are annealed. For example, the pairs can release at least one, two, or three diverse oligonucleotides. In one embodiment, the released diverse oligonucleotides encode one or more of: CDR1, CDR2, and CDR3 of an immunoglobulin variable domain. The diverse oligonucleotides can be released sequentially or concurrently.

In one embodiment, the diverse oligonucleotides include at least 10³, 10⁴, 10⁵, 10⁶, 10⁸, 10⁹, or 10¹⁰ different oligonucleotides. In one embodiment, each diverse oligonucleotide is less than 200, 120, 80, 70, 65, 60, 55, 50, 45, 40, or 35 nucleotides in length. The diverse oligonucleotides can be at least about 20, 25, 30, 35, 40, 45, 50, or 60 nucleotides in length. Each diverse oligonucleotide can be at least 40%, 50%, 60%, 70%, 80%, 90%, 95%, or 98% identical to at least another diverse oligonucleotide. For example, a diverse oligonucleotide can have 1, 2, 3, or at least 4 mismatches with respect to another diverse oligonucleotide.

In a preferred embodiment, each of the diverse oligonucleotides is of equal length as the others or are within 30, 20, 15, or 10% of the average length of the diverse oligonucleotides. In one embodiment, the diverse oligonucleotides of the plurality all have a length within 8, 6, 4, 3, 2, or 1 nucleotide of each other. Each of the diverse oligonucleotides can include 3′ and/or 5′ terminal regions of at least 6 nucleotides in length that are identical (or at least 70% identical) to corresponding terminal regions of each of the other diverse oligonucleotides. The terminal regions can be between 6 and 20 nucleotides in length, e.g., between 6 and 15, or 10 and 18 nucleotides-in length. In a preferred embodiment, the terminal regions are exactly complementary to a corresponding site on the template nucleic acid. In a preferred embodiment, each of the diverse oligonucleotides includes a sequence corresponding to (e.g., partially complementary to) a common region of the template (e.g., of at least 5 or 10 nucleotides). Each diverse oligonucleotide can include a naturally occurring sequence or a synthetic sequence. The diverse oligonucleotides can be constructed by chemical synthesis. In another embodiment, the diverse oligonucleotides are constructed by cleavage of a diverse nucleic acid strand.

In a preferred embodiment, each diverse oligonucleotide encodes a CDR or fragment thereof, e.g., a fragment including at least 5 amino acids. In a much preferred embodiment, the diverse oligonucleotides further include 3′ and/or 5′ terminal regions that anneal to a sequence that flanks a sequence encoding a CDR (or its complement), e.g., a sequence that encodes a framework region (or its complement), e.g., at least one, two, three, four, or five nucleotides thereof. The terminal regions are preferably less varied than the sequence between the terminal regions among the diverse oligonucleotides. The CDR can be a heavy chain CDR (e.g., heavy chain CDR1, CDR2, and CDR3) or a light chain CDR (e.g., light chain CDR1, CDR2, and CDR3). In a preferred embodiment the diverse oligonucleotides preferably do not include the entire sequence of the framework regions which flank the CDR, e.g., contain less than 2, 5, 8, 10, or 15 of the amino acids of each of the flanking framework regions.

In another preferred embodiment, each diverse oligonucleotide encodes an enzyme active site residue, e.g., a residue that is within 2 Angstroms of a bound substrate or cofactor.

In one embodiment, the diverse nucleic acids include at least 10³, 10⁴, 10⁵, 10⁶, 10⁸, 10⁹, or 10¹⁰ different nucleic acids. The diverse nucleic acids can be, e.g., mRNA, cDNA, or genomic nucleic acids. Each diverse nucleic acid can be fixed to a solid support. In a preferred embodiment, the diverse nucleic acids are obtained from a mammalian cell, e.g., a hematopoietic cell such as a B or T cell. In another preferred embodiment, the mammalian cell is obtained from a subject having an immune disorder. The diverse nucleic acids can be obtained from a mammalian cell cultured in vitro. The cell can also be stimulated to undergo somatic mutagenesis of immunoglobulin genes, class switching of immunoglobulin genes, or proliferation.

The template nucleic acid can encode a polypeptide of at least 10, 20, 50, 100, or 200 amino acids. For example, the polypeptide can include a domain of a cell surface protein, an enzyme, a T cell receptor, an MHC protein, a protease inhibitor, a scaffold domain, or a transcription factor. In one embodiment, the polypeptide does not include an immunoglobulin domain, i.e., the polypeptide is not an antibody.

In one embodiment, the polypeptide has a binding activity or is preselected for a binding activity. In another embodiment, the polypeptide has an enzymatic activity or is preselected for an enzymatic activity. The polypeptide can be naturally occurring or synthetic, e.g., partially synthetic, e.g., a synthetic variant of a naturally occurring polypeptide. The preselecting can include identifying the polypeptide from a display library on the basis of the binding activity.

In a preferred embodiment, the polypeptide includes an immunoglobulin domain, e.g., a variable domain, e.g., a VH or VL domain. The sequence can further include an immunoglobulin constant domain, e.g., a CH1 or CL. With respect to a VH domain, the template can further include a sequence encoding a CH2 and CH3 domain. The VH or VL domain can include a synthetic CDR or a germline CDR (e.g., a human CDR). Further, the VH or VL domain can include a framework region, e.g., a human framework region. For example, the polypeptide can include a VH and CH1 domain or a VL and CL domain. The polypeptide can include both a VH and VL domain, e.g., as a single-chain Fv domain (ScFv). The polypeptide can be such that the VH and VL domains form, e.g., Fab fragments, F(ab′)₂, Fv fragments, and single-chain Fv fragments. The polypeptides include an antigen binding site, e.g., a functional antigen binding site. In a preferred embodiment the template includes at least one, and preferably two or three CDRs, and all or part of at least one framework region. For example, it can include at least one CDR, e.g., a CDR1, and all or part of the framework regions which flank CDR1.

In another preferred embodiment, the template nucleic acid encodes a second polypeptide. The first and second polypeptide can form a complex, e.g., the first and second polypeptide can be non-covalently bound or covalently bound, e.g., by one or more disulfides. For example, the complex can include a Fab.

The combining can include annealing at least-some of the diverse oligonucleotides to the template nucleic acid strand. In one embodiment, the annealed oligonucleotides include diverse oligonucleotides of the subset and diverse oligonucleotides not of the subset to the template nucleic acid strand. Subsequent washing of the template nucleic acid strand dissociates the hybridized diverse oligonucleotides not of the subset. In another embodiment, the annealed diverse oligonucleotides are exclusively from the subset.

In one embodiment the conditions for the contacting include a temperature greater than 40° C. In another embodiment, the conditions include a temperature within 10 or 5° C. of a Tₘ, or a temperature greater than Tₘ-10°, Tₘ-5°, or Tₘ, wherein the Tₘ is the Tₘ of a segment of the template nucleic acid strand for its exact complement, and the segment is the region to which the diverse oligonucleotides hybridize. In one embodiment, the selected solution conditions are approximately a condition listed in Table 1. The hybridization conditions can include formamide or urea. The hybridization conditions can be selected so as to result in a preferred level of variation in the product, e.g., wherein the resulting molecules are at least 70, 80, 85, 90, 95, 97, or 98% homologous to the template. In some embodiments the level of homology is with regard to the entire length of the template, while in others it is with regard to the regions which correspond to diverse oligonucleotides.

In one embodiment, the template nucleic acid strand is limiting, and, for example, each diversity oligonucleotide of the population competes for the template nucleic acid strand under equilibrium binding conditions, e.g., conditions selected to favor competitive binding. In another embodiment, the template nucleic acid strand is not limiting. Exemplary molar ratios for the template nucleic acid strand to the diversity oligonucleotides include between 100:1 and 1:100; 10:1 and 1:10; 5:1 and 1:5; 10:1 and 1:1; 1:1 and 1:10.

In one embodiment, the subjecting includes separating at least some of the subset of diverse oligonucleotides that can anneal to the template nucleic acid strand from the remaining diverse oligonucleotides of the plurality. The separating can include washing the template nucleic acid strand. The template nucleic acid can be attached to a solid support. For example, the template nucleic acid strand can be immobilized on a solid support, e.g., by a covalent or non-covalent linkage. The washing conditions can be more stringent than conditions for the contacting. In another embodiment, the separating includes a size separation, e.g., using a membrane porous to unannealed diverse oligonucleotides but not annealed diverse oligonucleotides, a gel exclusion method, a sedimentation method, or an electrophoretic method.

In a preferred embodiment, a plurality of template nucleic acid strands are provided. The template nucleic acid strands of the plurality can differ from one another. For example, the template nucleic acid strands can be at least 50% (e.g., at least 60%, 70%, or 80%) identical to each other. The template nucleic acid strands can encode polypeptides that share the same scaffold domain. In a preferred embodiment, each template nucleic acid strand of the plurality encodes a polypeptide that has an activity or is preselected for an activity.

In one embodiment, each template strand or template strand complement encodes a polypeptide domain that, preferably, has at least a threshold activity. The method can further include screening the polypeptide encoded by the diversified strand complement (or a complement thereof), e.g., for an improved level of activity that exceeds the threshold activity. The threshold activity can be less than about 50, 10, 1, 0.1, or 0.01% of the improved level of activity. In a preferred embodiment, the one or more template nucleic acid strands is a plurality of template nucleic acid strands. In one embodiment, each template nucleic acid strand of the plurality of template nucleic acid strands is the same. In another embodiment, the plurality of template nucleic acid strands includes at least 2, 4, 8, 12, 30, 100, or 150 different template nucleic acid strands. In a preferred embodiment, the plurality of template nucleic acid strands includes different strands such that each or its complement encodes a polypeptide that includes a domain with at least a threshold activity of interest. For example, the strands of the plurality can include strands, each encoding a different polypeptide that is homologous (e.g., at least 40, 50, 60, 70, 80, 90, 95%) to the other encoded polypeptides, and/or has at least a threshold activity, e.g., a threshold measure of the same activity as the other polypeptides.

In another embodiment, the sequence of the template nucleic acid strand is not known at the time of the annealing. For example, the complete sequence of the template nucleic acid strand may be undetermined. More particularly, the sequence of the template nucleic acid strand in a region to which a diversity oligonucleotide can anneal is not known at the time of the annealing. An example of such a region is a region that encodes a CDR of an immunoglobulin variable domain.

In a preferred embodiment, the template nucleic acid(s) comprise DNA. In another embodiment, they comprise RNA.

The template strand can be linear or circular. In one embodiment, the template strand is immobilized on a solid support, e.g., using a covalent or non-covalent linkage. The template strand can include uracil at at least some nucleotides. The template strand can further include a unique restriction enzyme site, one or more selectable markers, e.g., one functional selectable marker and one marker that includes a lesion, one or more bacteriophage genes, e.g., a gene encoding a major or minor coat protein, e.g., filamentous phage gene III. Each template nucleic acid can be tagged or fixed to a solid support.

In another embodiment, the template nucleic strand includes a sequence encoding a transcription factor functional domain (e.g., for a two-hybrid assay), a cytotoxin, a label (e.g., green fluorescent protein or luciferase).

In one embodiment, the template strand comprises a promoter, e.g., a prokaryotic promoter, e.g., a bacteriophage promoter such as the T7, T3, or SP6 promoter. In another embodiment, the template strand includes a signal peptide, e.g., a eukaryotic or prokaryotic signal peptide.

In another embodiment, the template includes a nucleic acid sequence that encodes an enzyme or an inactivated enzyme, (e.g., as the sequence to be varied)

In one embodiment, the diversified nucleic acids are homologous (e.g., at least 30% homologous, more preferably at least about 40%, 50%, 60%, 70%, 80%, 90%, or more homologous) to one of the plurality of template nucleic acid strands. Preferably the diversified nucleic acids are homologous to each template nucleic acid strand of the plurality. In another embodiment, the diversified nucleic acids are homologous (e.g., at least 30% homologous, more preferably at least about 40%, 50%, 60%, 70%, or more homologous) to a reference domain, and each of the template nucleic acids is homologous to the reference domain.

In one embodiment, the annealed oligonucleotide is both extended and ligated. In another embodiment, the annealed oligonucleotide is extended. The extending and/or ligating can occur at least partially in a cell. Preferably, the extending and/or ligating occurs in vitro. The extending can be effected by a DNA polymerase or an RNA polymerase. Examples of DNA polymerases include E. coli polymerase I, T4 DNA polymerase, and reverse transcriptase (an RNA-dependent DNA polymerase). In a preferred embodiment, the DNA polymerase is a non-strand displacing DNA polymerase (e.g., T4 or T7 DNA polymerase). In another preferred embodiment, the DNA polymerase is a thermostable DNA polymerase. Another preferred DNA polymerase is the Klenow fragment of E. coli polymerase I or any DNA polymerase that lacks a 3′ to 5′ exonuclease activity.

In one embodiment, the method includes separating the diversified strand from the template strand. In another embodiment, the method includes separating diversified strand-template strand heteroduplexes from homoduplexes, e.g., using a mismatch binding protein. In another embodiment, the method can further include one or more of: amplifying the diversified strand, selectively disabling the template strand, and isolating the diversified strand.

The method can further include ligating the extended, hybridized diverse oligonucleotides. The method can include optionally introducing the diversified strand, a replicate, or complement thereof into cells, and/or optionally, translating the diversified strand, a replicate, or complement thereof.

In a preferred embodiment, the method further includes synthesizing a polypeptide encoded by the diversified strand or its complement. The translating can be in vitro or in vivo (i.e., in a host cell, e.g., a cultured cell or a transgenic cell that is part of an animal or plant). The host cell can be a prokaryotic cell (e.g., a bacterial cell) or is eukaryotic cell (e.g., a fungal cell, such as yeast, or a mammalian cell).

In one embodiment, the polypeptide is attached to the host cell surface (e.g., a yeast or mammalian cell surface, e.g., by means of a transmembrane protein or domain thereof or a peripheral membrane protein) or a virus surface, e.g., a filamentous phage coat protein or fragment thereof. The attachment can be direct or indirect (e.g., bridged), and can be covalent or non-covalent. In another embodiment, the polypeptide is attached to a solid support, e.g., a bead, particle, three-dimensional matrix, or planar array.

The method can further include constructing a library that includes the diversified strands, e.g., by introducing the diversified strand into a host cells with other diversified strands.

The method can further include screening the diversified strands or the complements thereof, e.g., using a method described herein. Exemplary methods include a display library, a polypeptide array, an in vitro assay, or an in vivo assay.

In another aspect, the invention features a method of providing a library of genetic packages that present an immunoglobulin protein. The method includes: a) providing a first plurality of genetic packages, each package comprising an accessible protein that comprises an immunoglobulin variable domain and varies among the plurality of genetic packages and a coding nucleic acid that encodes the accessible protein; b) contacting the first plurality of genetic packages to a target; c) separating genetic packages of the first plurality that bind to the target from genetic packages that do not bind to the target; d) preparing template nucleic acids from at least one of the separated genetic packages that bind to the target, the template nucleic acids comprising a sequence from the coding nucleic acid of the respective genetic packages; e) providing a plurality of diversity oligonucleotides that can anneal to at least some of the template nucleic acids and that each comprise a nucleic acid sequence encoding a single CDR and a portion of the flanking framework regions, or a complement thereof; e) combining the diversity oligonucleotides and the template nucleic acids in a mixture; f) subjecting the mixture to conditions such that only a subset of the plurality of diversity oligonucleotides can anneal to the template nucleic acids; g) extending and/or ligating an annealed oligonucleotide of the subset to form a plurality of altered nucleic acid strands that each incorporate a diversity oligonucleotide and a sequence complementary to one of the template nucleic acids; and h) preparing a second plurality of genetic packages from the altered nucleic acid strands or complements thereof as coding nucleic acids for the accessible protein component of each respective genetic package, thereby providing a library of genetic packages that present an immunoglobulin protein. The method can further include: i) contacting the second plurality of genetic packages to a target; and j) separating genetic package of the second plurality that bind to the target from genetic packages that do not bind to the target. The method can further include other features described herein.

In another aspect, the invention features a method that includes a) providing a first plurality of genetic packages, each package comprising an accessible protein that comprise a varied region that varies among the plurality of genetic packages and that is at least 8, 20, 30, 90, or 120 amino acids in length, and includes less than 100, 60, 50, or 31 varied amino acid positions and less than 40, 30, 20, or 5 invariant amino acid and a coding nucleic acid that encodes the accessible protein; b) contacting the first plurality of genetic packages to a target; c) separating genetic packages of the first plurality that bind to the target from genetic packages that do not bind to the target; d) preparing template nucleic acids from at least one of the separated genetic packages that bind to the target, the template nucleic acids comprising a sequence from the coding nucleic acid of the respective genetic packages; e) providing a plurality of diversity oligonucleotides that can anneal to at least some of the template nucleic acids at a site that overlaps (e.g., partially overlaps, or spans) the sequence encoding the varied region, or a complement thereof, wherein the diversity oligonucleotides include at least 10² different nucleic acids sequences; e) combining the diversity oligonucleotides and the template nucleic acids in a mixture; f) subjecting the mixture to conditions such that only a subset of the plurality of diversity oligonucleotides can anneal to the template nucleic acids; g) extending and/or ligating an annealed oligonucleotide of the subset to form a plurality of altered nucleic acid strands that each incorporate a diversity oligonucleotide and a sequence complementary to one of the template nucleic acids; and h) preparing a second plurality of genetic packages from the altered nucleic acid strands or complements thereof as coding nucleic acids for the accessible protein component of each respective genetic package, thereby providing a library of genetic packages that present a varied peptide sequence. The method can include other features describe herein.

In a related aspect, the invention features a method that includes a) providing a template nucleic acid or a plurality of template nucleic acids, each encoding a peptide of less than 31, 25, 21, or 15 amino acids that independently binds to a target molecule and a plurality of diversity oligonucleotides that can anneal to at least one of the one or more template nucleic acids at a site that overlaps (e.g., partially overlaps, or spans) a sequence encoding the peptide, wherein the diversity oligonucleotides include at least 10² different nucleic acids sequences; b) combining the diversity oligonucleotides and the one or more template nucleic acids in a mixture; c) subjecting the mixture to conditions such that only a subset of the plurality of diversity oligonucleotides can anneal to the one or more template nucleic acids; d) extending and/or ligating an annealed oligonucleotide of the subset to form a plurality of altered nucleic acid strands that each incorporate a diversity oligonucleotide and a sequence complementary to one of the template nucleic acids. The method can further include, for example, preparing a plurality of genetic packages from the altered nucleic acid strands or complements thereof as coding nucleic acids for the accessible protein component of each respective genetic package, thereby providing a library of genetic packages that present a varied peptide sequence. The peptide can be fused to other amino acid sequences, e.g., a linker and/or gene III protein. The method can include other features describe herein.

In another aspect, the invention features a method that includes: a) providing i) a display library comprising members that each display a polypeptide comprising an element of immunoglobulin variable domain and ii) diverse oligonucleotides; b) identifying members of the display library which display polypeptides that have at least a threshold degree of a given activity; c) providing (e.g., isolating) template nucleic acid strands for at least one of the identified members of the display library; d) providing diverse nucleic acids that each encode a immunoglobulin variable domain; e) annealing a cleavage-directing oligonucleotide to a plurality of members of the diverse nucleic acids to form cleavable regions; f) cleaving the cleavable regions to form a plurality of diverse oligonucleotides, wherein each of the diverse oligonucleotides encodes a sequence that includes a CDR and each diverse oligonucleotide is within 10% of the average length of all the diverse oligonucleotides; g) contacting the plurality of diverse oligonucleotides and the template nucleic acid strand in a mixture; h) subjecting the mixture to conditions such that only a subset of the plurality of diverse oligonucleotides can anneal to the template nucleic acid strand; and i) extending and/or ligating an annealed oligonucleotide of the subset to form a diversified strand that is partially complementary to the template nucleic acid strand. The element of an immunoglobulin variable domain can comprise, e.g., one or more CDRs, and/or one or more FR regions (or portions thereof), preferably at least one CDR and at least one portion of an FR region, e.g., at least 2, 3, 4, or 5 amino acids of one or both FR regions flanking the at least one CDR.

In one embodiment, the cleavage-directing oligonucleotide includes a stem-loop structure, e.g., a structure that includes a recognition site for a Type IIS restriction enzyme. The cleaving is effected by the Type IIS restriction enzyme. In another embodiment, the cleaving is effected by a Type II restriction enzyme, e.g., an enzyme hat recognizes a site of six basepairs, or less than six basepairs, e.g., five or four basepairs. In a preferred embodiment, the cleaving occurs at a temperature greater than 40° C.

In one embodiment, the cleavage-directing oligonucleotide forms a heteroduplex with the diverse nucleic acid and the cleavable region is fully complementary to the diverse nucleic acid within the heteroduplex. In a preferred embodiment, at least two cleavage-directing oligonucleotides are annealed to each of the diverse nucleic acids, e.g., one directs the cleavage of a 5′ terminus of a diverse oligonucleotide and the other directs the cleavage of a 3′ terminus of the diverse oligonucleotide. In a preferred embodiment, at least three pairs of cleavage-directing oligonucleotides are annealed. For example, the pairs can release at least one, two, or three diverse oligonucleotides. In one embodiment, the released diverse oligonucleotides encode one or more of: CDR1, CDR2, and CDR3 of an immunoglobulin variable domain. The diverse oligonucleotides can be released sequentially or concurrently.

In one embodiment, the diverse oligonucleotides include at least 10³, 10⁴, 10⁵, 10⁶, 10⁸, 10⁹, or 10¹⁰ different oligonucleotides. In one embodiment, each diverse oligonucleotide is less than 200, 120, 80, 70, 65, 60, 55, 50, 45, 40, or 35 nucleotides in length. The diverse oligonucleotides can be at least about 20, 25, 30, 35, 40, 45, 50, or 60 nucleotides in length. Each diverse oligonucleotide can be at least 40%, 50%, 60%, 70%, 80%, 90%, 95%, or 98% identical to at least another diverse oligonucleotide. For example, a diverse oligonucleotide can have 1, 2, 3, or at least 4 mismatches with respect to another diverse oligonucleotide.

In a preferred embodiment, each of the diverse oligonucleotides is of equal length as the others or are within 30, 20, 15, or 10% of the average length of the diverse oligonucleotides. In one embodiment, the diverse oligonucleotides of the plurality all have a length within 8, 6, 4, 3, 2, or 1 nucleotide of each other. Each of the diverse oligonucleotides can include 3′ and/or 5′ terminal regions of at least 6 nucleotides in length that are identical (or at least 70% identical) to corresponding terminal regions of each of the other diverse oligonucleotides. The terminal regions can be between 6 and 20 nucleotides in length, e.g., between 6 and 15, or 10 and 18 nucleotides in length. In a preferred embodiment, the terminal regions are exactly complementary to a corresponding site on the template nucleic acid. In a preferred embodiment, each of the diverse oligonucleotides includes a sequence corresponding to (e.g., partially complementary to) a common region of the template (e.g., of at least 5 or 10 nucleotides). Each diverse oligonucleotide can include a naturally occurring sequence or a synthetic sequence. The diverse oligonucleotides can be constructed by chemical synthesis. In another embodiment, the diverse oligonucleotides are constructed by cleavage of a diverse nucleic acid strand.

In a preferred embodiment, each diverse oligonucleotide encodes a CDR or fragment thereof, e.g., a fragment including at least 5 amino acids. In a much preferred embodiment, the diverse oligonucleotides further include 3′ and/or 5′ terminal regions that anneal to a sequence that flanks a sequence encoding a CDR (or its complement), e.g., a sequence that encodes a framework region (or its complement), e.g., at least one, two, three, four, or five nucleotides thereof. The terminal regions are preferably less varied than the sequence between the terminal regions among the diverse oligonucleotides. The CDR can be a heavy chain CDR (e.g., heavy chain CDR1, CDR2, and CDR3) or a light chain CDR (e.g., light chain CDR1, CDR2, and CDR3). In a preferred embodiment the diverse oligonucleotides preferably do not include the entire sequence of the framework regions which flank the CDR, e.g., contain less than 2, 5, 8, 10, or 15 of the amino acids of each of the flanking framework regions.

In another preferred embodiment, each diverse oligonucleotide encodes an enzyme active site residue, e.g., a residue that is within 2 Angstroms of a bound substrate or cofactor.

In one embodiment, the diverse nucleic acids include at least 10³, 10⁴, 10⁵, 10⁶, 10⁸, 10⁹, or 10¹⁰ different nucleic acids. The diverse nucleic acids can be, e.g., mRNA, cDNA, or genomic nucleic acids. Each diverse nucleic acid can be fixed to a solid support. In a preferred embodiment, the diverse nucleic acids are obtained from a mammalian cell, e.g., a hematopoietic cell such as a B or T cell. In another preferred embodiment, the mammalian cell is obtained from a subject having an immune disorder. The diverse nucleic acids can be obtained from a mammalian cell cultured in vitro. The cell can also be stimulated to undergo somatic mutagenesis of immunoglobulin genes, class switching of immunoglobulin genes, or proliferation. In a preferred embodiment, the diverse nucleic acids are obtained from a cDNA pool from B cells, e.g., human B cells, e.g., from a subject afflicted with peripheral blood syndrome, vasculitis, an autoimmune disorder, or a neoplastic disorder. For example, the method can further include reverse transcribing cDNA from mRNA isolated from B cells.

The template nucleic acid can encode a polypeptide of at least 10, 20, 50, 100, or 200 amino acids. For example, the polypeptide can include a domain of a cell surface protein, an enzyme, a T cell receptor, an MHC protein, a protease inhibitor, a scaffold domain, or a transcription factor. In one embodiment, the polypeptide does not include an immunoglobulin domain, i.e., the polypeptide is not an antibody.

In one embodiment, the polypeptide has a binding activity or is preselected for a binding activity. In another embodiment, the polypeptide has an enzymatic activity or is preselected for an enzymatic activity. The polypeptide can be naturally occurring or synthetic, e.g., partially synthetic, e.g., a synthetic variant of a naturally occurring polypeptide. The preselecting can include identifying the polypeptide from a display library on the basis of the binding activity.

In a preferred embodiment, the immunoglobulin domain variable domain comprises a VH or VL domain. The sequence can further include an immunoglobulin constant domain, e.g., a CH1 or CL. With respect to a VH domain, the template can further include a sequence encoding a CH2 and CH3 domain. The VH or VL domain can include a synthetic CDR or a germline CDR (e.g., a human CDR). Further, the VH or VL domain can include a framework region, e.g., a human framework region. For example, the polypeptide can include a VH and CH1 domain or a VL and CL domain. The polypeptide can include both a VH and VL domain, e.g., as a single-chain Fv domain (ScFv). The polypeptide can be such that the VH and VL domains form, e.g., Fab fragments, F(ab′)₂, Fv fragments, and single-chain Fv fragments. The polypeptides include an antigen binding site, e.g., a functional antigen binding site. In a preferred embodiment the template includes at least one, and preferably two or three CDRs, and all or part of at least one framework region. For example, it can include at least one CDR, e.g., a CDR1, and all or part of the framework regions which flank CDR1.

In another preferred embodiment, the template nucleic acid encodes a second polypeptide. The first and second polypeptide can form a complex, e.g., the first and second polypeptide can be non-covalently bound or covalently bound, e.g., by one or more disulfides. For example, the complex can include a Fab.

The combining can include annealing at least some of the diverse oligonucleotides to the template nucleic acid strand. In one embodiment, the annealed oligonucleotides include diverse oligonucleotides of the subset and diverse oligonucleotides not of the subset to the template nucleic acid strand. Subsequent washing of the template nucleic acid strand dissociates the hybridized diverse oligonucleotides not of the subset. In another embodiment, the annealed diverse oligonucleotides are exclusively from the subset.

In one embodiment the conditions for the contacting include a temperature greater than 40° C. In another embodiment, the conditions include a temperature within 10 or 5° C. of a Tₘ, or a temperature greater than Tₘ-10°, Tₘ-5°, or Tₘ, wherein the Tₘ is the Tₘ of a segment of the template nucleic acid strand for its exact complement, and the segment is the region to which the diverse oligonucleotides hybridize. In one embodiment, the selected solution conditions are approximately a condition listed in Table 1. The hybridization conditions can include formamide or urea. The hybridization conditions can be selected so as to result in a preferred level of variation in the product, e.g., wherein the resulting molecules are at least 70, 80, 85, 90, 95, 97, or 98% homologous to the template. In some embodiments the level of homology is with regard to the entire length of the template, while in others it is with regard to the regions which correspond to diverse oligonucleotides.

In one embodiment, the template nucleic acid strand is limiting, and, for example, each diversity oligonucleotide of the population competes for the template nucleic acid strand under equilibrium binding conditions, e.g., conditions selected to favor competitive binding. In another embodiment, the template nucleic acid strand is not limiting. Exemplary molar ratios for the template nucleic acid strand to the diversity oligonucleotides include between 100:1 and 1:100; 10:1 and 1:10; 5:1 and 1:5; 10:1 and 1:1; 1:1 and 1:10.

In one embodiment, the subjecting includes separating at least some of the subset of diverse oligonucleotides that can anneal to the template nucleic acid strand from the remaining diverse oligonucleotides of the plurality. The separating can include washing the template nucleic acid strand. The template nucleic acid can be attached to a solid support. For example, the template nucleic acid strand can be immobilized on a solid support, e.g., by a covalent or non-covalent linkage. The washing conditions can be more stringent than conditions for the contacting. In another embodiment, the separating includes a size separation, e.g., using a membrane porous to unannealed diverse oligonucleotides but not annealed diverse oligonucleotides, a gel exclusion method, a sedimentation method, or an electrophoretic method.

In a preferred embodiment, a plurality of template nucleic acid strands are provided. The template nucleic acid strands of the plurality can differ from one another. For example, the template nucleic acid strands can be at least 50% (e.g., at least 60%, 70%, or 80%) identical to each other. The template nucleic acid strands can encode polypeptides that share the same scaffold domain. In a preferred embodiment, each template nucleic acid strand of the plurality encodes a polypeptide that has an activity or is preselected for an activity.

In one embodiment, each template strand or template strand complement encodes a polypeptide domain that, preferably, has at least a threshold activity. The method can further include screening the polypeptide encoded by the diversified strand complement (or a complement thereof), e.g., for an improved level of activity that exceeds the threshold activity. The threshold activity can be less than about 50, 10, 1, 0.1, or 0.01% of the improved level of activity. In a preferred embodiment, the one or more template nucleic acid strands is a plurality of template nucleic acid strands. In one embodiment, each template nucleic acid strand of the plurality of template nucleic acid strands is the same. In another embodiment, the plurality of template nucleic acid strands includes at least 2, 4, 8, 12, 30, 100, or 150 different template nucleic acid strands. In a preferred embodiment, the plurality of template nucleic acid strands includes different strands such that each or its complement encodes a polypeptide that includes a domain with at least a threshold activity of interest. For example, the strands of the plurality can include strands, each encoding a different polypeptide that is homologous (e.g., at least 40, 50, 60, 70, 80, 90, 95%) to the other encoded polypeptides, and/or has at least a threshold activity, e.g., a threshold measure of the same activity as the other polypeptides.

In another embodiment, the sequence of the template nucleic acid strand is not known at the time of the annealing. For example, the complete sequence of the template nucleic acid strand may be undetermined. More particularly, the sequence of the template nucleic acid strand in a region to which a diversity oligonucleotide can anneal is not known at the time of the annealing. An example of such a region is a region that encodes a CDR of an immunoglobulin variable domain.

In a preferred embodiment, the template nucleic acid(s) comprise DNA. In another embodiment, they comprise RNA.

The template strand can be linear or circular. In one embodiment, the template strand is immobilized on a solid support, e.g., using a covalent or non-covalent linkage. The template strand can include uracil at at least some nucleotides. The template strand can further include a unique restriction enzyme site, one or more selectable markers, e.g., one functional selectable marker and one marker that includes a lesion, one or more bacteriophage genes, e.g., a gene encoding a major or minor coat protein, e.g., filamentous phage gene III. Each template nucleic acid can be tagged or fixed to a solid support.

In another embodiment, the template nucleic strand includes a sequence encoding a transcription factor functional domain (e.g., for a two-hybrid assay), a cytotoxin, a label (e.g., green fluorescent protein or luciferase).

In one embodiment, the template strand comprises a promoter, e.g., a prokaryotic promoter, e.g., a bacteriophage promoter such as the T7, T3, or SP6 promoter. In another embodiment, the template strand includes a signal peptide, e.g., a eukaryotic or prokaryotic signal peptide.

In another embodiment, the template includes a nucleic acid sequence that encodes an enzyme or an inactivated enzyme, (e.g., as the sequence to be varied)

In one embodiment, the diversified nucleic acids are homologous (e.g., at least 30% homologous, more preferably at least about 40%, 50%,-60%, 70%, 80%, 90%, or more homologous) to one of the plurality of template nucleic acid strands. Preferably the diversified nucleic acids are homologous to each template nucleic acid strand of the plurality. In another embodiment, the diversified nucleic acids are homologous (e.g., at least 30% homologous, more preferably at least about 40%, 50%, 60%, 70%, or more homologous) to a reference domain, and each of the template nucleic acids is homologous to the reference domain.

In one embodiment, the annealed oligonucleotide is both extended and ligated. In another embodiment, the annealed oligonucleotide is extended. The extending and/or ligating can occur at least partially in a cell. Preferably, the extending and/or ligating occurs in vitro. The extending can be effected by a DNA polymerase or an RNA polymerase. Examples of DNA polymerases include E. coli polymerase I, T4 DNA polymerase, and reverse transcriptase (an RNA-dependent DNA polymerase). In a preferred embodiment, the DNA polymerase is a non-strand displacing DNA polymerase (e.g., T4 or T7 DNA polymerase). In another preferred embodiment, the DNA polymerase is a thermostable DNA polymerase. Another preferred DNA polymerase is the Klenow fragment of E. coli polymerase I or any DNA polymerase that lacks a 3′ to 5′ exonuclease activity.

In one embodiment, the method includes separating the diversified strand from the template strand. In another embodiment, the method includes separating diversified strand-template strand heteroduplexes from homoduplexes, e.g., using a mismatch binding protein. In another embodiment, the method can further include one or more of: amplifying the diversified strand, selectively disabling the template strand, and isolating the diversified strand.

The method can further include ligating the extended, hybridized diverse oligonucleotides. The method can include optionally introducing the diversified strand, a replicate, or complement thereof into cells, and/or optionally, translating the diversified strand, a replicate, or complement thereof.

In a preferred embodiment, the method further includes synthesizing a polypeptide encoded by the diversified strand or its complement. The translating can be in vitro or in vivo (i.e., in a host cell, e.g., a cultured cell or a transgenic cell that is part of an animal or plant). The host cell can be a prokaryotic cell (e.g., a bacterial cell) or is eukaryotic cell (e.g., a fungal cell, such as yeast, or a mammalian cell).

In one embodiment, the polypeptide is attached to the host cell surface (e.g., a yeast or mammalian cell surface, e.g., by means of a transmembrane protein or domain thereof or a peripheral membrane protein) or a virus surface, e.g., a filamentous phage coat protein or fragment thereof. The attachment can be direct or indirect (e.g., bridged), and can be covalent or non-covalent. In another embodiment, the polypeptide is attached to a solid support, e.g., a bead, particle, three-dimensional matrix, or planar array.

The method can further include constructing a library that includes the diversified strands, e.g., by introducing the diversified strand into a host cells with other diversified strands.

The method can further include screening the diversified strands or the complements thereof, e.g., using a method described herein. Exemplary methods include a display library, a polypeptide array, an in vitro assay, or an in vivo assay.

In another aspect, the invention features a method of providing an oligonucleotide. The method includes: a) providing a nucleic acid (or a plurality of diverse nucleic acids) that is attached to a solid support and that includes a single-stranded region; b) annealing a first cleavage-directing oligonucleotide to the nucleic acid to form a first double-stranded segment; c) cleaving the first double-stranded segment to release a first fragment from the solid support and a first-cleaved nucleic acid attached to the support; d) annealing a second cleavage-directing oligonucleotide to the first-cleaved nucleic acid to form a second double-stranded segment; e) cleaving the second double-stranded segment to release a second fragment from the support and a second-cleaved subject nucleic acid attached to the support; f) isolating the second fragment from the support thereby providing the oligonucleotide.

The invention also features a related method which provides a pool of diverse oligonucleotides. The method includes: a) providing a plurality of diverse nucleic acids, each nucleic acid of the plurality being attached to a solid support and including a single-stranded region; b) annealing a first cleavage-directing oligonucleotide to each nucleic acid of the plurality to form first double-stranded segments; c) cleaving the first double-stranded segments to release first fragments from the solid support and first-cleaved nucleic acids attached to the support; d) annealing a second cleavage-directing oligonucleotide to each of the first-cleaved nucleic acids to form second double-stranded segments; e) cleaving the second double-stranded segments to release second fragments from the support and second-cleaved subject nucleic acids attached to the support; f) isolating the second fragments from the support thereby providing a pool of diverse oligonucleotides.

The method can further include annealing the second fragment to a template nucleic acid and extending the second fragment. In a preferred embodiment, the first and/or second oligonucleotide includes a double-stranded segment that is recognized by a Type IIS enzyme.

In a preferred embodiment, the cleaving of the first and/or second double-stranded segment occurs at a temperature greater than 40° C., e.g., at least 45, 50, 55, or 60° C.

The invention also features reaction mixtures, reaction intermediates, and kits applicable for the method described herein. For example, the invention features a kit that includes a first, second, and third container. The first container includes a repertoire of diversity oligonucleotides from a natural source of CDR1, the second container includes a repertoire of diversity oligonucleotides from a natural source of CDR2, and the third container includes a repertoire of diversity oligonucleotides from a natural source of CDR3.

The term “random” describes an event whose outcome is almost entirely due to chance given the conditions. For example, the addition of a nucleotide to an oligonucleotide from a mixture of nucleotides is a chance event relative to the particular proportions of concentrations of nucleotides in the mixture. “Non-random” describes an event whose outcome is not determined almost entirely by chance. For example, the selection of an oligonucleotide from a mixture of oligonucleotides during a low-stringency hybridization to a given template nucleic acid is a non-random event.

An “oligonucleotide” is a polynucleotide of between 8 and 300 nucleotides in length, preferably less than 100 nucleotides.

A “cleavage-directing oligonucleotide” is a polynucleotide of less than 300 nucleotides, more preferably less than 100 nucleotides, and most preferably less than 50 nucleotides, and that includes at least a single-stranded segment which can anneal to a nucleic acid strand that includes a region complementary to the single-stranded segment such that a duplex (e.g., heteroduplex or homoduplex) formed by the annealing is cleavable by a site-specific endonuclease.

“Diverse nucleic acids” are a population of at least two nucleic acids that differ by at least one nucleotide. In a preferred embodiment, the population includes at least 50 unique members, e.g., at least 10^{3, 10}⁴, 10⁵, 10⁶, 10⁸, 10⁹, or 10¹⁰ unique members, or ranges therebetween. In one embodiment, at least some of the members of the population are at least 30% identical to each other. For example, the population can be a population of nucleic acid sequences that encode variable domains.

As used herein, “diverse oligonucleotides” are a population of at least two polynucleotides of less than 300 nucleotides that differ from one another by at least one nucleotide. In a preferred embodiment, the population includes at least 50 unique members, e.g., at least 10³, 10⁴, 10⁵, 10⁶, 10⁸, 10⁹, or 10¹⁰ unique members, or ranges therebetween. The population can be random or non-random with respect to sequence diversity. Further, the sequence diversity can be natural or synthetic in origin. In one embodiment, a diverse oligonucleotide includes a non-natural nucleotide.

As used herein, a “library” is a collection of diverse nucleic acids, preferably in a replicable form. In a preferred embodiment, it includes at least 50 unique members, e.g., at least 10³, 10⁵, 10⁶, 10⁸, 10⁹, 10¹¹, or 10¹² unique members, or ranges therebetween. The library can encode polypeptides which can be translated from nucleic acids of the library. The library can include functional nucleic acids that do not encode a polypeptide. Replication can occur in a cell, e.g., the library can be maintained in a vector nucleic acid that includes an origin of DNA replication. Replication can occur in vitro, e.g., using PCR. For example, the library nucleic acids can include an oligonucleotide binding site. A “display library” is a collection of entities; each entity includes an accessible polypeptide component and a recoverable component that encodes or identifies the peptide component. Examples of display libraries are described below. A “replicable genetic package” or “genetic package”, as used herein, refers to an entity having a genetic component, e.g. an RNA or DNA component, which encodes all or part of a polypeptide which is attached to the genetic package and accessible to a probe, e.g., a probe attached to an insoluble support. The polypeptide is heterologous to the genetic package. The polypeptide can be covalently or non-covalently attached to the replicable display package, e.g. it can be attached to an endogenous component of the genetic package (e.g., a phage coat protein domain or a cell surface protein domain), or the nucleic acid component itself (e.g., a DNA-protein fusion). The heterologous polypeptide can be fused (e.g., as a translational fusion) to the endogenous component, or attached by a non-peptide bond (e.g., a disulfide bond).

The term “phage” and “bacteriophage” refer to replicable bacteriophage particles, e.g., particles that include a phage genome or modified phage genome as well as particles that include a phagemid nucleic acid (e.g., an episome with a phage packaging signal, which may or may not include endogenous phage genes).

The term “polypeptide” refers to a polymer of three or more amino acids linked by a peptide bond. The polypeptide may include one or more unnatural amino acids. Typically, the polypeptide includes only natural amino acids. The term “peptide” refers to a polypeptide that is between three and thirty-two amino acids in length. A “protein” can include one or more polypeptide chains. Accordingly, the term “protein” encompasses polypeptides and peptides. A protein or polypeptide can also include one or more modifications, e.g., a glycosylation, amidation, phosphorylation, and so forth.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that a preparation of a polypeptide of interest is at least 10% pure (e.g., at least 20, 50, 70, 80, 90, 95% pure). The term “isolated nucleic acid molecule” or “purified nucleic acid molecule” includes nucleic acid molecules that are separated from other nucleic acid molecules present in the natural source of the nucleic acid. An “isolated” nucleic acid molecule, such as a cDNA molecule, can be substantially free of other cellular material, or culture medium when produced by recombinant techniques, or substantially free of chemical precursors or other chemicals when chemically synthesized.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology” specifically where numerical values of identity or homology are recited). The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available from the Genetics Computer Group, WI USA), using the Blossum 62 matrix, and a gap weight of 12 and a length weight of 4.

An “epitope” refers to the site on a target compound that is bound by a ligand, e.g., a peptide ligand or an antigen-binding ligand (e.g., a Fab or antibody). In the case where the target compound is a protein, for example, an epitope may refer to the amino acids that are bound by the ligand.

“Binding affinity” refers to the apparent association constant or Kₐ. The Kₐ is the reciprocal of the dissociation constant or K_{d}. A ligand-binding polypeptide may, for example, have a binding affinity of at least 10⁻⁵, 10⁻⁶, 10⁻⁷ or 10⁻⁸ M for a particular target molecule. Higher affinity binding of a ligand to a first target relative to a second target can be indicated by a higher Kₐ¹ (or a smaller numerical value K_{d}¹) for binding the first target than the Kₐ² (or numerical value K_{d}²) for binding the second target. In such cases the ligand has specificity for the first target relative to the second target. Binding affinity can be determined by a variety of methods including equilibrium dialysis, equilibrium binding, gel filtration, ELISA, or spectroscopy (e.g., using a fluorescence assay). These techniques can be used to measure the concentration of bound and free ligand as a function of ligand (or target) concentration. The concentration of bound ligand ([Bound]) is related to the concentration of free ligand ([Free]) and the concentration of binding sites for the ligand on the target where (N) is the number of binding sites per target molecule by the following equation:

[Bound]=N·[Free]/((1/Ka)+[Free])

It is possible to screen a population of nucleic acids varied by a method described herein for at least a minimal binding affinity, e.g., at least 10⁻⁵, 10⁻⁶, 10⁻⁷ or 10⁻⁸ M. The screening and variation can be repeated until at least a selected threshold binding affinity is achieved.

The following terms may be abbreviated: nucleotide=nt; single-stranded DNA=ssDNA; cleavage directing oligonucleotide=CDO; complementarity determining region=CDR; framework region=FR; RACE=rapid amplification of cDNA ends.

Controlling definitions with respect to immunoglobulin domains and related terms are provided below in the section entitled “Antibody Maturation.”

Other features and advantages of the invention will be apparent from the following detailed description, and from the claims. All patents and references cited herein are incorporated in their entirety by reference.

### DESCRIPTION OF THE DRAWINGS

FIG. 1 is a schematic of one embodiment of a method for cleaving diverse nucleic acids to create diverse oligonucleotides.

FIG. 2 is a schematic of one embodiment of the hybridization-controlled variation method.

FIG. 3A is a schematic of an exemplary method of preparing CDR pools by double cleavage-directing oligonucleotide (CDO)-mediated cleavage. The method includes providing an immobilized single stranded DNA obtained from a V-gene pool by RACE. As shown, the ssDNA includes a biotinylated terminus that is bound to immobilized streptavidin (S). As described in Example 5, oligonucleotides complementary with the regions bordering the CDR region of interest (in this figure, CDR1), are used to direct the cleavage at specific sites surrounding the CDRs of all templates that have sufficient homology with the oligonucleotides. If a diverse source of starting variable region genes is used, the result is a pool of CDR encoding ssDNAs, which can be used in a subsequent hybridization reaction.

FIGS. 3B, 3C, and 3D are a schematic of an exemplary method of preparation of Vkappa CDR1, 2, 3 respectively. With a similar principle as in FIG. 3A, Vkappa ssDNA is cleaved twice using oligonucleotides complementary with regions bordering the CDR of interest (‘adaptors’ or ‘cleavage-directing oligonucleotides, CDOs). This time the orientation has been reversed compared to FIG. 3A, which produces the reverse CDR strand. When this method is used, the CDR ssDNA produced can be directly used for hybridization to template ssDNA produced from antibody genes cloned into phage and phagemid vectors such as DY3F31. The sizes given are for an exemplary template; when repertoires are cleaved, fragments of different sizes will be generated.

FIGS. 3E, 3F, and 3G are schematics of an exemplary method of preparation of Vlambda CDR1, 2, 3 respectively. See also legend of FIG. 3B. For cleavage of the Vλ-CDR3, a mix of CDO's was used (“adaptor mix”); described in Example 10.

FIGS. 4A and 4B: Human Vλ-CDR1 preparation with CJ-cleavage with BstNI and BstEII. Using a double CDO-mediated cleavage and the preparation strategy depicted in FIG. 3, a CDR1 encoding ssDNA molecule was prepared from a single V-lambda template (derived from a clone isolated from a phage display library of human antibodies). The 802 nt fragment was cut first with BstNI, to obtain fragments of 531 nt and 262 nt. The latter fragment was retrieved from the beads (FIG. 4A). Lanes 1-3 Starting material to beads; 4-6)SN after BstNI digest; 7-8) Left on beads after BstN1. This fragment was than cut with BstEII, yielding a fragment of 188 nt left on the beads, and 74 nt coming off the beads and thus easily recovered (FIG. 4B).

FIG. 5: Preparation of a pool of lambda CDR1 regions via PCR. Using suitably designed oligonucleotides for binding to the FR1 or FR2 region of human V-lambda-I genes, the CDR1 region was amplified. A DNA preparation of human lambda light chains (L race) prepared according to example 4, or DNA from a single antibody clone, F2, was subjected to PCR with two different oligonucleotide-sets (left and right panel respectively). Lanes are 1) L race, 5 μl; 2)L race, 1 μl; 3) F2, 5 μl; 4) F2, 1 μl; 5) L race, 5 μl; 6) L race, 1 μl; 7)F2, 5 μl; 8) F2, 1 μl;

FIG. 6 is a schematic of an exemplary mutagenesis procedure for use with CDR pools. The method shows steps: Clone gene of interest into M13; Transform into dut ung strain; Isolate uracil-containing ssDNA; Anneal mutagenic primer; Synthesize second strand with T7 DNA Polymerase and T4 DNA ligase; Transform into WT strain Parental strand is not replicated.

FIG. 7: Analysis of mutations in a Vκ1 template, hybridization at various temperatures. Controlled-hybridization mutagenesis was carried out at the calculated Tₘ for hybridization for clone A11, which is a human Fab binding to streptavidin and utilizing a light chain of the Vκ1 family. This segment has the amino acid sequence RASQSISSYLN (SEQ ID NO:46). The CDR1 is completely germline (GL012; also SEQ ID NO:46). Controlled-hybridization mutagenesis was carried out using a pool of CDRs derived by double CDO-mediated cleavage of human B-cell derived kappa genes. The CDR fragments had 10 residues overlap in the FR1 region, 33 within the CDR regions, and 18 in FR2 (indicated by 10/33/18). Clones resulting from the mutagenesis using three hybridization temperatures were sequenced. Shown is a compilation of the mutations found in the resulting clone. An overview of the frequency of clones with mutations is as follows: Hybridization Tₘ: 71.6° C. ->9/20 (45%) of clones with newly formed strand; ->8/9 (89%) mutations introduced. Hybridization Tₘ: 69.6° C.: ->11/20 (55%) of clones with newly formed strand; ->3/11 (27%) mutations introduced. Hybridization Tₘ: 68.2° C.: ->15/19 (79%) of clones with newly formed strand and ->9/14 (64%) mutations introduced. Method: ssDNA of human Fab clone A11 in phage vector DY3F31 (with uracil; Kunkel method) was hybridized to a CDR1 fragment derived from a natural Vk pool, and the mutant strand rescued.

Clone No. 1 is SEQ ID NO:47, and so forth to No. 58 which is SEQ ID NO:62.

FIG. 8: Controlled-Hybridization Mutagenesis of a clone of the Vλ1 family. Controlled-Hybridization mutagenesis was applied to a template Vλ1 V-gene, of a human anti-streptavidin antibody clone F2, with CDR1 sequence indicated (top, left). Mutations were introduced using various conditions for hybridization, and clones obtained with the Kunkel mutagenesis procedure described in examples 13-16. Shown is a compilation of the resulting amino acids mutations found in the resulting mutant F2 clones. Clone F2 is based on the germline segment 1e; the CDR1 is completely germline, as indicated by the dashes (top, left). Most of the other lambda1-family germ lines (1f to 1b) have a shorter CDR1 region (indicated by the * at the position of the deletion, bottom, left). Controlled-hybridization mutagenesis captures somatic mutations introduced via hybridization with CDRs encoded by the same germ line, with up to 8 changes from the original sequence (top, right). Pending hybridization conditions, Controlled-hybridization mutagenesis can also lead to the replacement of the CDR1 of the clone by other relatively homologous germlines, possibly in combination with somatic mutations in the CDR1 or bordering FR regions (bottom, right).

Clone F2 and GL-le include the peptide sequence TGSSSNIGAGYDVH (SEQ ID NO:63). Clone No. GL-1f is SEQ ID NO:64, and so forth to No. GL-1b which is SEQ ID NO:69. On the right side, sequences refer respectively to SEQ ID NO:70 to SEQ ID NO:81.

FIG. 9: Analysis of mutations in a Vλ1 template, hybridization at the calculated Tₘ. Controlled-hybridization mutagenesis was carried out at the calculated Tₘ for hybridization for the F2 clone, 73.5° C., using a pool of CDRs derived by double CDO-mediated cleavage of human B-cell derived lambda genes. The CDR fragments had 10 residues overlap in the FR1 region, 42 within the CDR regions, and 18 in FR2 (indicated by 10/42/18). Clones resulting from the mutagenesis were sequenced. Shown is a compilation of the mutations found in the resulting clones, with on the right indicated the number of nucleotide changes (including deletions). Under these conditions, 77% of mutated clones are derived of the same germline as the starting template F2 (le).

Clone No. A2 is SEQ ID NO:82, and so forth to No. AW48 which is SEQ ID NO:94.

FIG. 10: Analysis of mutations in a Vλ1 template, hybridization below the calculated Tₘ. Controlled-hybridization mutagenesis was carried out at below the calculated Tₘ for hybridization for the F2 clone, chosen was 60° C., using a pool of CDRs derived by double CDO-mediated cleavage of human B-cell derived lambda genes. The CDR fragments had 10 residues overlap in the FR1 region, 42 within the CDR regions, and 18 in FR2 (indicated by 10/42/18). Clones resulting from the mutagenesis were sequenced. Shown is a compilation of the mutations found in the resulting clones, with on the right indicated the number of nucleotide changes (including deletions). Under these conditions, the majority of the clones have obtained a CDR1 sequence related to another germ line (indicated by the frequent deletion of three nucleotides; see also FIG. 5 for other germ line sequences).

Clone No. 1.6 is SEQ ID NO:95, and so forth to No. 11.5 which is SEQ ID NO:113.

### DETAILED DESCRIPTION

The invention provides, in part, a method of generating controlled mutations in a template nucleic acid sequence. The template serves as a guide for the improvement. The new variants that are generated can be screened for an improved property.

Diverse oligonucleotides are hybridized to the template nucleic acid. The hybridization of the diverse oligonucleotides is typically sensitive to the number of mismatches between the diverse oligonucleotides and the template. The hybridization conditions are controlled as required. For example, they can be chosen to favor few or many mismatches between the template and the diverse oligonucleotides.

In some cases, the use of hybridization to control mutation avoids the untempered discard of critical features of a nucleic acid sequence in an attempt to exchange them for other, potentially better features. As the diverse oligonucleotides must hybridize to the template nucleic acid under the controlled conditions, at least some of the original template nucleic acid sequence is retained. Generally, the diversified sequence is at least 50, 60, 70, 80, 90, 95 or 98% identical to the template.

The use of an identified or preselected template sequence to query a pool of enriching sequences can obviate the need for the custom synthesis of new oligonucleotides to alter a particular template. Further, each template serves as its own guide for diversification. Thus, multiple different templates can be independently diversified within the same reaction mixture. Efficient mutagenesis can result in a large proportion of new sequences that include a variation relative to the initial template.

One embodiment of the variation method includes the following modules: identifying a repertoire for diversity, producing diverse oligonucleotides, annealing diverse oligonucleotides using hybridization control, separating annealed oligonucleotides, synthesizing a diversity strand removing the template strands, and screening a library of diversity strands.

Repertoires for Diversity

The method relies on diverse oligonucleotides as a source of variation. The method then introduces the sequences provided by these oligonucleotides into the template nucleic acid. The sequences for diverse oligonucleotides can be obtained from a variety of sources.

Natural Sources. In a preferred embodiment, the oligonucleotides originate from a natural source. The natural source can be obtained from an intermediary source such as a library that serves as a repository of natural sequences.

The natural source can be genomic nucleic acid from a single species or multiple species. For example, within a single species, nucleic acids that encode families of related polypeptide domains or polypeptides can be used. Between species, nucleic acids that encode analogs of a polypeptide domain or polypeptide can be used. Further, both these spectra of diversity (e.g., intra- and inter-species diversity) can be used together.

Some additional examples of natural sources include immune cells, e.g., naïve immune cells of mammals, e.g., humans, primates, or rodents. Nucleic acids that encode immunoglobulin variable domains and T cell receptor domains can be isolated from these cells. For example, diverse oligonucleotides can be obtained from nucleic acid segments that encode the CDR regions of such domains. Further examples of obtaining diverse oligonucleotides from immune cells are provided below.

Another natural source is an environmental sample, e.g., a soil or water sample that includes diverse microorganisms. Nucleic acid is prepared from the sample. Primers that recognize the conserved nucleic acid features can be used to amplify a diverse pool of related nucleic acids from the different microorganisms that are in the sample. A pool of nucleic acids can be amplified from nucleic acid prepared from the sample. For example, degenerate primers that anneal to conserved regions of a nucleic acid encoding an enzyme can be used to amplify a pool of nucleic acids that encode different species variants of the enzyme.

The nucleic acid from a natural source can be a RNA (e.g., mRNA), cDNA, genomic DNA, or organelle DNA.

Preselected Sources. Diverse oligonucleotides can be obtained from a preselected source. The preselected source can be, e.g., a group of hits from the initial screen of a diversity library. For example, a diverse library of mutants of particular enzyme can be screened to identify thermostable variants of the enzyme. Diverse oligonucleotides are then obtained from the pool of thermostable variants. These diverse oligonucleotides can be used to introduce variations that increase the thermostability of a polypeptide while maintaining another property, e.g., substrate-specificity.

Random and Designed Synthetic Sources. Diverse oligonucleotides can include repertoires of random and designed synthetic sources. One exemplary repertoire of randomized oligonucleotides includes oligonucleotides that include randomized segments or oligonucleotides that are totally randomized. For example, the oligonucleotides synthesizers can produce segments that are “NNN” or “NNK” in order to create diverse oligonucleotides for varying nucleic acids that encode polypeptides. Other mixtures of nucleotide precursors can be used to restrict diversity to smaller quadrants of the codon table. In addition, activated trinucleotides can be used as subunits for constructing synthetic nucleic acids (see, e.g., Virnekas et al. (1994) Nucl Acids Res 22:5600-7). Oligonucleotides are synthesized on a solid phase support, one codon (i.e., trinucleotide) at a time. The trinucleotide or codon includes an activated phosphoramidite. This approach enables the synthesis of a nucleic acid that at a given position can encoded a selected number of amino acids. The frequency of these amino acids can be regulated by the proportion of codons in the mixture.

In some embodiments, the diversity segments are synthesized between constant regions or regions of lesser diversity. These constant regions can function to anchor the diverse oligonucleotide to the template nucleic acid. However, in order to provide at least some hybridization control during the annealing of the diverse oligonucleotides, the length and composition of the anchor segments are tailored such that the sequence of the diversity segments impacts whether the diverse oligonucleotide is annealed. The length and composition of the anchor segments can be tailored empirically or by estimating their contribution to the Tₘ of the diverse oligonucleotide, e.g., using methods described herein and as known in the art.

Diverse oligonucleotides can also be pooled from individual oligonucleotides. For example, a set of oligonucleotides can be designed with the assistance of computer software. The software can be used to maintain similar Tₘ (of each oligonucleotide for its exact complement) and to sample particular regions of sequence space. Typically, the set of oligonucleotides is designed so that it can be used to introduce variations into multiple different template nucleic acids that are related, e.g., related by the sequence of the scaffold.

The individual oligonucleotides can be synthesized using automated oligonucleotide synthesizers or in parallel on a planar solid support (e.g., as described herein). A diverse pool can be designed to include a controlled degree of variation, and at particular positions. The pool can then be used for a variety of different template nucleic acid sequences. Hybridization control provides a second level of control on the extent of variation. For example, the pool can be generally designed to vary the active site of serine proteases. The diverse oligonucleotides are designed to include sequences that can anchor, e.g., to highly conserved catalytic residues, while introducing diversity in the vicinity of these residues.

Constructing Diverse Oligonucleotides

Once a source of diverse oligonucleotides is selected, the diverse oligonucleotides are constructed. A variety of methods are available to construct the diverse oligonucleotides.

Initial Design. The construction is based in part on the intended usage of the diverse oligonucleotides. One exemplary design includes anchor regions at the 3′ and 5′ termini of the diverse oligonucleotides, and a central segment. In this case, the diverse oligonucleotides vary to the greatest extent in the central segment, and to a much lesser extent in the anchor regions.

The anchor regions can be designed to be complementary to an intended template—nucleic acid or a consensus sequence for likely template nucleic acids. Most preferably, the ultimate and/or penultimate nucleotide of the 3′ anchor region is exactly complementary to the intended template nucleic acid so that the 3′ anchor region can be easily extended.

Another feature of the design is that all the diverse oligonucleotides align with the same region of the intended template nucleic acid, e.g., they all overlap the same target site. Preferably, each is within 30, 20, or 10% of the average length. Preferably, they include substantially the same anchor regions.

PCR Amplification. PCR can be used to amplify diverse oligonucleotides. Forward and reverse primers are designed to anneal to conserved regions that flank a region of diversity, e.g., the anchor regions. The forward primer can be tagged, e.g., so that is bindable to a solid support. The forward and reverse primers are then used in a PCR amplification reaction using a diverse set of nucleic acids as the PCR template molecules. The forward primer can be present in excess over the reverse primer. The PCR products are then bound tog the solid support. The two strands of the PCR products are denatured, and the strands primed by the reverse primer are washed from the solid support. The strands primed by the forward primer are then released from the support, e.g., by breaking the bond between the forward primer and the tag. The released strands are isolated and, thus, available for use as a diverse oligonucleotides.

Oligonucleotide-Directed Cleavage. In a preferred embodiment, the variation method includes using an oligonucleotide to direct cleavage of nucleic acid strands that are a source of diversity. Generally, the oligonucleotide directs cleavage by the formation of a duplex (e.g., a homo- or hetero-duplex) between a single stranded region of the oligonucleotide and a single stranded region of a nucleic acid that is a source of diversity.

Some exemplary methods for such cleavage are described in U.S. Ser. No. 09/837,306, filed Apr. 17, 2001 and WO 01/79481. The methods can be used to cleave a homoduplex or heteroduplex formed by an individual single-stranded nucleic acid and a cleavage-directing oligonucleotide. The method can also be used to cleave homo- or heteroduplexes formed by a plurality of differing, yet related single-stranded nucleic acids that are a source of diversity and one or more cleavage-directing oligonucleotides. In one embodiment, the method enables natural sources of diversity to be readily accessed. For example, a population of diverse oligonucleotides can be excised from the sources and used to vary a template nucleic acid.

An exemplary application of the method to nucleic acids encoding immunoglobulin domains is described herein below.

In one embodiment, a single-stranded cleavage-directing oligonucleotide is used to form a restriction enzyme cleavage site, e.g., a site for a Type II restriction enzyme. The site can be, e.g., 6 nucleotides or less in length, e.g., 6, 5, or 4 basepairs in length. The method includes: (i) annealing the single-stranded cleavage-directing oligonucleotide to a subject nucleic acid to form a double-stranded region that includes a cleavable site; and (ii) cleaving the double-stranded region at the cleavable site, e.g., using a restriction endonuclease.

In a preferred embodiment, if the subject nucleic acid is an entirely single-stranded nucleic acid, the contacting and the cleaving steps are performed at a chosen temperature sufficient to maintain the subject nucleic acid in substantially single-stranded form in regions to which the cleavage-directing oligonucleotide does not anneal. Thus, the formation of hairpins and other secondary structures that may fortuitously include a recognition site for the restriction enzyme is prevented.

In another preferred embodiment, the cleavage-directing oligonucleotide is functionally complementary to the nucleic acid over a large enough region to allow the two strands to associate such that cleavage may occur at the chosen temperature and at the desired location, and the cleavage is carried out using a restriction endonuclease that is active at the chosen temperature. In a preferred embodiment, the cleavage is performed at a temperature of greater than 40° C., e.g., at least 40, 45, 50, 55, or 60° C. For example, the temperature can be between 40-65° C. or 45-60° C. In another embodiment, the cleavage is performed at a temperature of less than 40° C., e.g., an ambient temperature or a low temperature.

The use of oligonucleotides to form local double-stranded regions that include a restriction endonuclease recognition site allows sites that are well-positioned but not unique in the subject nucleic acid to be exploited. From a plurality of potential sites for a restriction endonuclease, typically only one particular site is rendered cleavable (i.e., double-stranded) by the annealing of the cleavage-directing oligonucleotide.

In a preferred embodiment, the subject nucleic acids are a source of diversity, e.g., the subject nucleic acids vary from each other. They can be obtained from a natural or synthetic source, e.g., as described elsewhere herein.

The cleavage-directing oligonucleotides are designed such that they direct cleavage at the same corresponding position in a substantial fraction of the subject nucleic acids in a diversity population. In one embodiment, a plurality of different cleavage-directing oligonucleotides is used so that an even more substantial fraction (e.g., at least 80%, 90%, or 99%) of the subject nucleic acids of the diversity population are cleaved.

Design of the cleavage-directing oligonucleotides can be done using computer software that analyzes nucleic acid sequences for restriction enzyme sites. The software can be configured to analyze a plurality of subject nucleic acid sequences and identify one or more sites that enable a substantial fraction of the sequences to be cleaved at the same corresponding position. For example, the software can tally the number of subject nucleic acid sequences that include a particular site at a particular position, and display to a user the percentage of sequences that would be cleaved by the use of the restriction endonuclease that recognizes the particular site. The user can also specify a window, e.g., of 30 to 50 nucleotides within one of the subject nucleic acid sequence or within an alignment of the sequences. The software then searches for a restriction enzyme or set of restriction enzymes that cleaves a substantial fraction of the subject nucleic acid sequences within the window.

In another embodiment of the oligonucleotide-directed cleavage method, the cleavage-directing oligonucleotide includes a double-stranded region, e.g., the cleavage-directing oligonucleotide includes a stem-loop structure. The stem forms a double stranded region which includes a recognition site for a Type IIS restriction endonuclease. The cleavage-directing oligonucleotide also includes a single-stranded region which can anneal to a single-stranded region of a subject nucleic acid to form a double-stranded region in which the Type IIS restriction endonucleases cleaves.

Cleavage-directing oligonucleotides that include a double-stranded region that has a Type II restriction endonuclease recognition site and a single-stranded region are termed “lollipop oligonucleotides,” herein. These lollipop oligonucleotides allow cleavage of any specific sequence of sufficient length and complexity since the single-stranded segment of a lollipop oligonucleotide can be programmed to hybridize to the intended target sequence. Accordingly, the cleaved site can be non-palidromic. On the one hand, these oligonucleotides enable specific and precise cleavage with respect to the location of the cleavage site. On the other hand, they also enable flexibility as they can tolerate mismatches (e.g., 1 to 3 mismatches) in the region of hybridization between the lollipop oligonucleotide and the target sequence. For some applications, this flexibility reduces bias in resultant products.

The sequence of the single-stranded DNA adapter or overlap portion of the lollipop oligonucleotide typically consists of about 14-22 bases. However, longer or shorter adapters may be used. The size depends on the ability of the adapter to associate with its functional complement in the single-stranded DNA and the temperature used for contacting the lollipop oligonucleotide and the single-stranded DNA at the temperature used for cleaving the DNA with the type IIS enzyme. The adapter must be functionally complementary to the single-stranded DNA over a large enough region to allow the two strands to associate such that the cleavage may occur at the chosen temperature and at the desired location. The single-stranded or overlap portions are preferably 14-20 bases, and more preferably 18-20 bases in length.

The site chosen for cleavage using the lollipop oligonucleotide is preferably one that is present in a substantial fraction of the subject nucleic acids. The sites can be non-palindromic, naturally occurring, or synthetic. In another embodiment, a plurality of lollipop oligonucleotides are used, e.g., if a single oligonucleotide is not sufficient to cleave a substantial fraction of the subject nucleic acids.

As described above, the double-stranded portion of the lollipop oligonucleotide includes a Type IIS endonuclease recognition site. Any Type IIS enzyme that is active at a temperature necessary to maintain the single-stranded DNA substantially in that form and to allow the single-stranded segment of the lollipop oligonucleotide to anneal long enough to the single-stranded DNA to permit cleavage at the desired site may be used.

The preferred Type IIS enzymes for use with lollipop oligonucleotides provide asymmetrical cleavage of the single-stranded DNA. Examples of such enzymes include: AarI, AceIII, Bbr7I, BbvI, BbvII, Bce83I, BceAI, BcefI, BciVI, BfiI, BinI, BscAI, BseRI, BsmFI, BspMI, EciI, Eco57I, FauI, FokI, GsuI, HgaI, HphI, MboII, MlyI, MmeI, MnlI, PleI, RleAI, SfaNI, SspD5I, Sth132I, StsI, TaqII, Tth111II, and UbaPI. One preferred Type IIS enzyme is FokI.

When a lollipop oligonucleotide having the FokI recognition site is used, for example, conditions can include one or more of: 1) excess of the lollipop oligonucleotide over target DNA present; 2) an activator of dimerization of the FokI enzyme; 3) a temperature between 45°-75° C., preferably above 50° C. and most preferably above 55° C.

Further examples illustrating the design of lollipop oligonucleotides can be found in U.S. Ser. No. 09/837,306, filed Apr. 17, 2001 and WO 01/79481. The lollipop oligonucleotides are designed to release a population of diverse oligonucleotides from a pool of diverse nucleic acids. The released diverse oligonucleotides can have substantially homogeneous termini and lengths.

In a preferred embodiment, after oligonucleotide-directed cleavage, the nucleic acid fragments generated by the cleavage are isolated. For example, one (or both) of the fragments generated by a single cleavage event can be used as a diverse oligonucleotide.

The cleavage reaction mixture can electrophoresed in a preparative gel that includes 6-16% acrylamide, 4 to 8M urea, and a gel running buffer such as 1×TBE (see, e.g., Chapter 10 In Sambrook & Russell (2001) Molecular Cloning: A Laboratory Manual, 3^{rd} Edition, Cold Spring Harbor Laboratory. After electrophoresis, the diverse oligonucleotides are then excised from the gel based on their length. In another example, diverse oligonucleotides can also be purified from the cleavage reaction mixture using HPLC or by low stringency hybridization to a complementary probe that is specific for the region to which the diverse oligonucleotides hybridize and excludes flanking sequences.

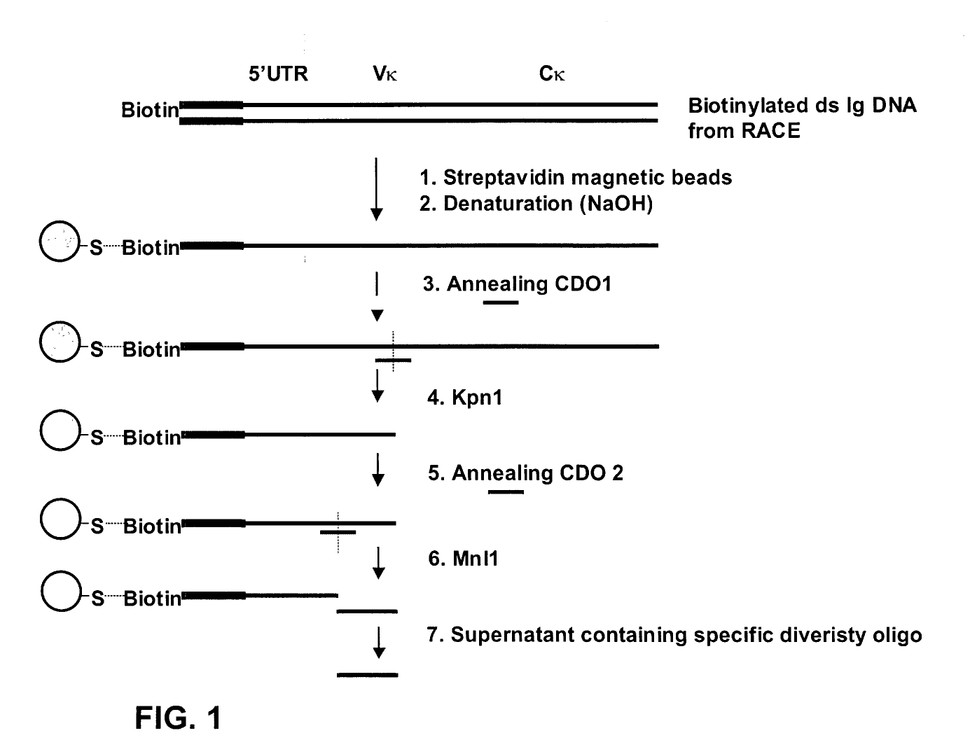

In another preferred embodiment, diverse oligonucleotides are separated by using sequential oligonucleotide-directed cleavage events on subject nucleic acids that are linked to a solid support, e.g., as diagrammed in FIG. 1.

Automated Oligonucleotide Synthesis. The diverse oligonucleotides can be synthesized, e.g., using automated oligonucleotide synthesizers. The synthesizers can be programmed to produce oligonucleotides that include at particular positions: a particular nucleotide, a mixture of nucleotides, a mixture of trinucleotides (or other oligomers), or an artificial nucleotide. The synthesizers typically use 3′ phosphoramidite-activated and 5′-protected subunits (e.g., nucleotides or trinucleotides) to sequentially add the subunits (or oligomers) to a growing nucleotide polymer coupled to a solid support.

In some embodiments, the diverse oligonucleotides include artificial bases. Some exemplary artificial bases include the “universal nucleotides” 3-nitropyrrole 2′-deoxynucloside and 5-nitroindole 2′-deoxynucleoside (5-nitroindole), and other nitro and cyano-substituted pyrrole deoxyribonucleotides (see, e.g., U.S. Pat. No. 5,780,233 and Loakes (2001) Nucleic Acids Res. 29:2437-2447). Other examples of artificial bases include inosine, the pyrimidine analogue 6H,8H-3,4-dihydropyrimido[4,5-c][1,2]oxazin-7-one, and the purine analogue N6-methoxy-2,6-diaminopurine.

Array-Based Synthesis. Oligonucleotides can also be synthesized on a planar solid support, e.g., using photolithography (see, e.g., U.S. Pat. No. 5,143,854, Fodor et al. (1991) Science 251:767-773; Fodor et al. (1993) Nature 364:555-556) or ink-jet printing (see, e.g., U.S. Pat. No. 5,474,796). These oligonucleotide synthetic methods can be programmed to produce a large number of diverse individual oligonucleotides. After synthesis, the oligonucleotides are released from the array, e.g., using a chemical treatment or an enzyme. The released oligonucleotides are pooled for the diversification method described herein.

Despite their length, diverse oligonucleotides created by any method described herein can be amplified (e.g., using PCR) or cloned into a plasmid for storage. For example, the diverse oligonucleotide can be inserted into a vector nucleic acid such that it can be released using a Type IIS restriction enzyme that generates flush ends.

Hybridization Control

Methods of the invention exploit hybridization control to control the degree of variation introduced into a template sequence. Hybridization is driven by hydrogen bonding between complementary DNA strands. The stability of a hybrid is determined in part by the solution conditions, the number of G-C basepairs, and the length of the hybrid. Mismatches between the two strands of the hybrid duplex (i.e., a heteroduplex) however, are destabilizing relative to a homoduplex formed of two strands that are entirely complementary, one of the two being either strand of the heteroduplex.

Hydrogen bonds between opposing, complementary bases—adenine and thymidine or guanine and cytosine—are consistent with the geometry of the double helix formed by two nucleic acid strands, particularly the B-form structure of double-stranded DNA. Mismatches are formed by opposing bases which are not complementary. For purine-purine and pyrimidine-pyrimidine pairs, the mismatches distort the double helical structure. Non-complementary purine-pyrimidine pairs can also form, but unlike complementary pairs, these pairs are unable to form the optimal hydrogen bonds available to complementary pairs.

The stability of any given hybrid can be measured or represented by the melting temperature (Tₘ) of the hybrid. The Tₘ is the temperature at which 50% of a given oligonucleotide is hybridized to its complementary strand, forming a hybrid. Typically, sequences with higher GC content have a higher Tₘ. Base-stacking interactions also affect the Tₘ, but to a lesser extent.

Tₘ is dependent on the solution conditions of the hybridization reaction. Tₘ increases with ionic strength since some cations bind preferentially to double-stranded duplexes.

Hybrid stability is also dependent on the presence or absence of destabilizing agents such as urea or formamide. Formamide is an ionizing solvent that can be used in aqueous buffers. The extent of depression of the Tₘ as a function of formamide can be estimated using equations described in Bolton and McCarthy (1962) Proc. Natl. Acad. Sci. USA 48:1390. For a GC content of between 30 and 75%, the Tₘ is depressed approximately 0.63° C. for each percentage of formamide.

The concentration of the nucleic acid strands of the hybrid is also a factor. Crowding agents such as polyethylene glycol and dextran sulfate can favor hybridization. For example, hybridization can be performed in solution conditions of about 2-20% dextran sulfate or 2 to 10% polyethylene glycol (PEG) 8000.

Quaternary ammonium salts can be used to accelerate the hybridization reaction.

Hybridization conditions can be selected to conform to the amount of variation desired. If little variation is desired, stringent conditions are used. If much variation is desired, reduced stringency conditions are used.

Under standard conditions, the Tₘ of a sequence for its perfect complement can be estimated using one of three equations. First, the “Wallace Rule” can be used to calculate the Tₘ of polynucleotides of about 15 to 20 nucleotides in length in conditions of about 1M NaCl or 6×SSC (Thein and Wallace (1986) In Human Genetic Diseases: A Practical Approach (ed. K. E. Davies), pages 33-50, IRL Press, Oxford, UK). (1×SSC is 0.15M NaCl and 15 mM sodium citrate)

Tₘ=2·(A+T)+4·(G+C) (1)

Second, the Baldino estimation provides the Tₘ of polynucleotides less than about 100 nucleotides in length, at cation concentrations of about 0.5 M, and GC contents of between 30 to 70% (Baldino et al. (1989) Methods Enzymol. 168:761-777; Bolton and McCarthy (1962) Proc. Natl. Acad. Sci. USA 48:1390). One preferred version of the Baldino estimation is set forth in Equation 2.

$${T}_{m} = 81.5 + 16.6 \cdot {log}_{10} [ Na + ] + 0.41 \cdot gc - \frac{500}{L} - \cdot 1.5 m - 0.63 f \qquad ( 2 )$$

wherein [Na+] is the concentration of sodium, gc is the percentage of G-C content, L is the length, m is the percentage of mismatches, and f is the percentage of formamide. This equation predicts the effect of mismatches by estimating a depression in Tₘ of 1.5° for each percentage of mismatch for a given polynucleotide.

Third, an accurate estimation of Tₘ can be determined by considering sequence-related thermodynamic data and nearest-neighbor interactions. Wetmur (1991) Crit. Rev. Biochem. Mol. Biol. 26:227-259 provides details of such a calculation. The nearest-neighbor interactions are significant with respect to mismatches as each mismatched region reduces the number of nearest neighbor interactions by two.

In cases where a narrow range of variation is required, it is possible to tune the hybridization conditions in order to control the range of variation. The template nucleic acid is combined with the pool of diversity oligonucleotides in replicates that each have the same hybridization solution. The diversity oligonucleotides are hybridized to the template at different temperatures. The annealed diverse oligonucleotides are extended to form diversity strands, which are cloned and sequenced. The extent of mutation is then determined from the sequence information to identify the temperature that provides the desired degree of mutation. This empirical method can also be applied to determine the extent of variation using different solutions conditions (e.g., at constant temperature).

Exemplary hybridization conditions are listed in Table 1. Notably, in some embodiments, discrimination is achieved after hybridization by washes at judiciously chosen conditions. Initially diverse oligonucleotides are annealed with little discrimination. Then, the less stable hybrids are dissociated using the stringent washes. Aliquots can be taken at intervals during an incremental washing process of increasing stringency. Each aliquot, then, includes hybrids for the formation of diversified strands of progressively less degree of variation.

In one preferred embodiment, the lengths of the diverse oligonucleotides used in a particular mutagenesis are similar, e.g., within 5 nucleotides of one another. Most preferably, they are the same length. Provided that all the diverse oligonucleotides have similar GC content, the Baldino equation (equation 2), indicates that all oligonucleotides would have similar Tₘ's for their perfect complements. Under these conditions, the number of mismatches provides the predominating control on the affinity of the diverse oligonucleotide for the template nucleic acid.

TABLE 1
Exemplary Hybridization Conditions

| Hybrid | Hybridization Temperature | Wash Temperature |
|---|---|---|
| Length (bp) | and Buffer | and Buffer |
| ≧50 | 65° C.; 1xSSC | 65° C.; 0.3xSSC |
| ≧50 | 42° C.; 1xSSC, 50% formamide | 65° C.; 0.3xSSC |
| <50 | Tₘ−5° C.; 1xSSC | Tₘ−5° C.; 1xSSC |
| ≧50 | 65° C.; 4xSSC -or- | 65° C.; 1xSSC |
| ≧50 | 42° C.; 4xSSC, 50% formamide | 65° C.; 1xSSC |
| <50 | Tₘ−5° C.; 4xSSC | Tₘ−5° C.; 4xSSC |
| ≧50 | 50° C.; 4xSSC | 50° C.; 2xSSC |
| ≧50 | 40° C.; 6xSSC, 50% formamide | 50° C.; 2xSSC |
| <50 | Tₘ−5° C.; 6xSSC | Tₘ−5° C.; 6xSSC |
| — | Tₘ+2° C.; | Tₘ+2° C.; |
| | 100 mM NaCl 50 mM | 100 mM NaCl 50 mM |
| | Tris-HCl 10 mM MgCl₂ | Tris-HCl 10 mM MgCl₂ |
| — | Tₘ−5° C.; | Tₘ−5° C.; |
| | 100 mM NaCl 50 mM | 100 mM NaCl 50 mM |
| | Tris-HCl 10 mM MgCl₂ | Tris-HCl 10 mM MgCl₂ |

In some implementations, the length of the diverse oligonucleotides is less than 90, 80, 70, or 60 nucleotides. Longer nucleic acids are generally more stable. Accordingly, the ability to control the number of hybridizing mismatches can be less for longer diverse oligonucleotides.

Separation of Unhybridized Diverse Oligonucleotides

In some embodiments, it is preferable to remove the unhybridized diverse oligonucleotides prior to extending hybridized diverse oligonucleotides. A variety of methods can be used to separate hybridized and unhybridized diverse oligonucleotides.

In a preferred embodiment, the template nucleic acid strand can be bound to an entity, e.g., an insoluble entity, e.g., a filter or particle. The insoluble entity can facilitate separation.

In a preferred embodiment, the template nucleic acid strand is tagged. The template is bound to a solid support using the tag, e.g., before, during, or after hybridization with the diverse oligonucleotides. Unhybridized diverse oligonucleotides are washed from the support, whereas the hybridized diverse oligonucleotides are retained. The solid support can be, e.g., a glass slide, a particle, or a filter.

In another preferred embodiment, the mixture is dialyzed to remove small nucleic acid fragments.

In still another preferred embodiment, the mixture is centrifuged against a dialysis membrane, e.g., using a Centricon filter. Small, unhybridized nucleic acid fragments pass through the membrane whereas the template nucleic acids and hybridized diverse oligonucleotides do not. The size of the pores in the membrane is judiciously chosen.

In another embodiment, the mixture of template nucleic acids is bound to a matrix that has affinity for nucleic acids. The matrix is selected such that small nucleic acid fragments, i.e., unhybridized oligonucleotides, do not bind efficiently to the matrix, whereas larger nucleic acids, e.g., template nucleic acids and hybridized oligonucleotides do.

If unhybridized oligonucleotides are not removed, the method can include extending the hybridized oligonucleotides using a DNA polymerase that is functional at the selective hybridization conditions of the reaction.

Diversity Strand Formation

After controlled hybridization of the diverse oligonucleotides, the annealed diverse oligonucleotides are extended using a nucleic acid polymerase, typically a DNA polymerase, e.g., a DNA-dependent DNA polymerase or an RNA-dependent DNA polymerase. Preferably, the DNA polymerase lacks 5′ to 3′ and/or 3′ to 5′ exonuclease activity. Most preferably the DNA polymerase lacks strand displacement activity. For example, T4 and T7 DNA polymerases do not significantly displace oligonucleotides when they advance and contact a previously annealed sequence. In a preferred embodiment, the DNA polymerase is T4 or T7 DNA polymerase. In another embodiment, the DNA polymerase is thermostable.

If the template nucleic acid is linear, an oligonucleotide is annealed to the 3′ most terminus of the template nucleic acid. Typically, the template nucleic acid is designed such that the 3′ end is constant regardless of differences that may be present in the remainder of the template nucleic acid. For example, the 3′ end may be a vector sequence that is the same regardless of the character of the sequence being mutagenized. The oligonucleotide that anneals to the 3′ most terminus is similarly extended.

If the template is circular, the diverse oligonucleotide may be sufficient to synthesize the diversity strand. Additional oligonucleotides can be used if it is necessary to increase the efficiency of priming the diversity strand.

A DNA ligase is then used to join the extended oligonucleotides. In the case where one oligonucleotide is extended on a circular template, the DNA ligase can be used to join the two ends. In a preferred embodiment, the DNA ligase is T4 DNA ligase. In another embodiment, the DNA ligase is a thermostable DNA ligase.

Removal of Template Strand

If desired, any one of a variety of methods can be used to efficiently recover the strand that incorporates variations from the diverse oligonucleotide (i.e., the diversity strand). Many available methods eliminate or reduce recovery of the template strand. Some examples include the following.

Uracil Incorporation. In this approach, the template strand includes uracil, e.g., uracil at a substantial number positions in substitution for thymidine. The uracil marked strand can be synthesized in a dut ung mutant E. coli strain, e.g., following the method of Kunkel (Kunkel (1985) Proc. Natl. Acad. Sci. USA 18:3439; U.S. Pat. No. 4,873,192). After a complementary strand is synthesized incorporating the diverse oligonucleotide, the duplex is transformed into an ung+ E. coli strain. Ung encodes a uracil N-glycosylase which digests uracil containing DNA strands. The transformed duplex is modified by the uracil N-glycosylase such that only the complementary strand that includes the diverse oligonucleotide is propagated.

In another embodiment, the uracil N-glycosylase treatment is effected in vitro, e.g., prior to a PCR reaction to prevent a template nucleic acid strand from being amplified.

Enhanced Antibiotic Resistance. In this approach, diverse oligonucleotides and a specialized oligonucleotide are annealed to the template strand. The template strand includes an ampicillin resistance gene. The specialized oligonucleotide anneals to the ampicillin resistance gene and alters the enzyme specificity of the encoded resistance factor. A diverse oligonucleotide anneals elsewhere on the template (i.e., in the region encoding a polypeptide being varied). Both oligonucleotides are extended to form a new strand—the diversity strand—which incorporates the mutations introduced by both oligonucleotides. When the two oligonucleotides are transformed into a host bacterial cell and the cell is grown in the presence of cefotaxime or ceftazidime, the diversity strand is selected for and is propagated. See, e.g., U.S. Pat. No. 5,780,270.

Restriction Site Disruption. The template strand can include a unique non-essential restriction enzyme cleavage site. The unique non-essential cleavage site is not cleavable while the template stand is single-stranded. The template removal method can include annealing a mismatch oligonucleotide that anneals to the restriction enzyme cleavage site. The mismatch oligonucleotide is extended concurrent with the diverse oligonucleotide. The mismatch oligonucleotide forms a heteroduplex with the template strand such that the restriction enzyme site is not cleavable, e.g., the mismatch is with a recognition or cleavage site of the restriction enzyme site. After the annealed diverse oligonucleotide and the mismatch oligonucleotide are extended, the reaction is digested with the restriction enzyme, e.g., to digest template strands to which the mismatch oligonucleotide did not anneal. The undigested heteroduplexes are then transformed into a repair defective (e.g., mutS) E. coli strain. See, e.g., U.S. Pat. No. 5,354,670; the Transformer™ protocol of Clontech, CA, USA; and the Chameleon® protocol of Stratagene, CA, USA.

Solid Phase Attachment. The template strand can be tagged, e.g., with a thiol. Before or after extension of the diverse oligonucleotides, the template strand can be fixed to a solid support. For example, the support can include a thiol that can form a disulfide bond to the thiol tag on the template strand. After the diversity strand is synthesized, the strands are denatured, and the diversity strand is washed from the support.

Still other method of removing the template strand include Eckstein's phosphorothioate technique (see, e.g., Chapter 5 of In Vitro Mutagenesis Protocols (1996) Ed. Trower, Humana Press),

Libraries: Construction and Expression

The diversity strands can be cloned into a vector, such as a plasmid or viral vector, e.g., to form a library of diverse nucleic acids. The vector may further comprise regulatory sequences, including for example, a promoter, operably linked to the nucleic acid(s) of interest. Large numbers of suitable vectors and promoters are known to those of skill in the art and are commercially available for generating the recombinant constructs. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, and pRIT5 (Pharmacia). Eukaryotic: pWLneo, pSV2cat, pOG44, PXTI, pSG (Stratagene) pSVK3, pBPV, pMSG, and pSVL (Pharmacia). One preferred class of preferred libraries is the display library, which is described below.

Methods well known to those skilled in the art can be used to construct vectors containing a polynucleotide described herein and appropriate transcriptional/translational control signals. These methods include in vitro recombinant DNA techniques, synthetic techniques and in vivo recombination/genetic recombination. See, for example, the techniques described in Sambrook & Russell, Molecular Cloning: A Laboratory Manual, 3^{rd} Edition, Cold Spring Harbor Laboratory, N.Y. (2001); Ausubel et al., Eds., Short Protocols in Molecular Biology: A Compendium of Methods from Current Protocols in Molecular Biology, Fifth Edition, Wiley N.Y. (2002) and Ausubel et al., Current Protocols in Molecular Biology, Greene Publishing Associates and Wiley Interscience, N.Y. (1989). Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P, and trc. Eukaryotic promoters include CMV immediate early, HSV thymidine kinase, early and late SV40, LTRs from retrovirus, mouse metallothionein-I, and various art-known tissue specific promoters.

Generally, recombinant expression vectors will include origins of replication and selectable markers permitting transformation of the host cell, e.g., the ampicillin resistance gene of E. coli and S. cerevisiae auxotrophic markers (such as URA3, LEU2, HIS3, and TRP1 genes), and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The polynucleotide can be assembled in appropriate phase with translation initiation and termination sequences, and preferably, a leader sequence capable of directing secretion of translated protein into the periplasmic space or extracellular medium. Optionally, a nucleic acid can encode a fusion protein including an N-terminal identification peptide imparting desired characteristics, e.g., stabilization or simplified purification of expressed recombinant product. Useful expression-vectors for bacteria are constructed by inserting a coding polynucleotide described herein together with suitable translation initiation and termination signals, optionally in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacteria can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and pGEM1 (Promega, Madison, Wis., USA).

The present invention further provides host cells containing the vectors, e.g., including a coding nucleic acid (e.g., varied by a method described herein), wherein the nucleic acid has been introduced into the host cell using known transformation, transfection or infection methods. For example, the host cells can include members of a library constructed from the diversity strand. The host cell can be a eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected, for example, by calcium phosphate transfection, DEAE, dextran mediated transfection, or electroporation (Davis, et al., Basic Methods in Molecular Biology (1986)).

Any host/vector system can be used to identify or characterize one or more of the regulatory elements that may be used in an implementation or to express a varied coding nucleic acid, e.g., as described herein. Exemplary host systems include, but are not limited to, eukaryotic hosts such as HeLa cells, CV-1 cell, COS cells, and Sf9 cells, as well as prokaryotic host such as E. coli and B. subtilis. Transgenic animals (e.g., Drosophila, C. elegans, mice, rats, goats, cows, and so forth) that express a varied coding nucleic acid can also produced.

The host of the present invention may also be a yeast or other fungus. In yeast, a number of vectors containing constitutive or inducible promoters may be used. For a review see, Current Protocols in Molecular Biology, Vol. 2, Ed. Ausubel et al., Greene Publish. Assoc. & Wiley Interscience, Ch. 13 (1988); Grant et al., Expression and Secretion Vectors for Yeast, in Methods in Enzymology, Ed. Wu & Grossman, Acad. Press, N.Y. 153:516-544 (1987); Glover, DNA Cloning, Vol. 11, IRL Press, Wash., D.C., Ch. 3 (1986); Bitter, Heterologous Gene Expression in Yeast, in Methods in Enzymology, Eds. Berger & Kimmel, Acad. Press, N.Y. 152:673-684 (1987); and The Molecular Biology of the Yeast Saccharomyces, Eds. Strathem et al., Cold Spring Harbor Press, Vols. 1 and 11 (1982).

The host cell may also be a prokaryotic cell such as E. coli, other enterobacteriaceae such as Serratia marescans, bacilli, various pseudomonads, or other prokaryotes which can be transformed, transfected, infected.

The present invention further provides host cells genetically engineered to contain polynucleotides described herein. For example, such host cells may contain nucleic acids introduced into the host cell using known transformation, transfection or infection methods. The present invention still further provides host cells genetically engineered to express polynucleotides that are in operative association with a regulatory sequence heterologous to the host cell which drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell.

Introduction of the recombinant construct into the host cell can be effected by calcium phosphate transfection, DEAE, dextran mediated transfection, or electroporation (Davis, L. et al., Basic Methods in Molecular Biology (1986)). The host cells containing one of polynucleotides, described herein, can be used in conventional manners to produce the gene product encoded by the isolated fragment (in the case of an ORF).

Any host/vector system can be used to express one or more of the diversity strands. These include, but are not limited to, eukaryotic hosts such as HeLa cells, CV-1 cell, COS cells, and Sf9 cells, as well as prokaryotic host such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated by reference.

Various mammalian cell culture systems can also be employed to express recombinant protein.

Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and also any necessary ribosome-binding sites, polyadenylation site, splice donor and acceptor sites, transcriptional termination sequences, and 5′ flanking nontranscribed sequences.

DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. In some embodiments, the template nucleic acid also encodes a polypeptide tag, e.g., penta- or hexa-histidine. The recombinant polypeptides encoded by a library of diversity strands can then be purified using affinity chromatography.

Microbial cells employed in expression of proteins can be disrupted by any convenient method, including freeze-thaw cycling, sonication, mechanical disruption, or use of cell lysing agents. A number of types of cells may act as suitable host cells for expression of the protein.

Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods. In another embodiment, cells and tissues may be engineered to express an endogenous gene comprising the polynucleotides described herein under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods.

Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting, including polyadenylation signals. mRNA stability elements, splice sites, leader sequences for enhancing or modifying transport or secretion properties of the protein, or other sequences which alter or improve the function or stability of protein or RNA molecules.

Additional guidance for preparing nucleic acid libraries, including display libraries (e.g., phage display libraries) and libraries encoding immunoglobulins can be found in WO 01/79481, WO 00/70023, PCT US02/12405, U.S. Ser. No. 09/837,306, filed Apr. 17, 2001; U.S. Ser. No. 10/045,674, filed Nov. 25, 2001; and U.S. Ser. No. 09/968,899, filed Nov. 19, 2001.

Screening Strategies

The process of controlled variation described here can be coupled to a variety of methods or systems. Some exemplary applications are as follows:

Secondary Screens. The methods described herein can be applied as a secondary screen after an initial screen. Generally, every screening method has a physical limitation on the number of sequences that can be sampled. The limitation may be imposed by transformation efficiency or the sheer mass of molecules required to explore every possible sequence. For example, sampling all possible polypeptides that are 50 amino acids in length would require sampling 20⁵⁰ or about 10⁶⁵ sequences. This is the equivalent of approximately 10⁴¹ moles of polypeptide.

One practical approach to sampling large portions of sequence space is to perform sequential screens. The sequential screens can be combined with a number of strategies. The strategies themselves are non-exclusive.

With respect to a first strategy, an initial screen is used to sparsely sample a large fraction of sequence space. The initial screen can rely on relatively low stringency or low threshold criteria for identifying hits among the sampled sequences. These hits identify local regions of the sequence space that have potential. The variation method described here is then used to more densely sample these local regions. The precise density of sampling that radiates from the region of each hit is controlled by the hybridization conditions.

With respect to a second strategy, an initial screen varies determinants that are known or suspected to have a primary role in a function of interest. These determinants are limited in number, but diversified maximally in order to reasonably sample possible combinations that have potential for the function. Then, the variation method is applied to introduce additional variations that might further improve or “mature” the properties of the initial hits. The method is particularly useful when the additional variations are in proximity or overlapping with the determinants. Hybridization control is used to introduce variations that tend to retain residues at the position of the determinants while not being completely constrained to the initially selected residues at these positions.

With respect to a third strategy, an initial screen identifies hits that are potentially useful. The variation method is then used to introduce-variation from a pre-selected repertoire, e.g., a repertoire which is independently known to have particular properties. For example, the naïve repertoire of nucleic acid sequences encoding immunoglobulin variable domains is one rich source of limited diversity. Another exemplary repertoire is a library of nucleic acids that encode proteins that have been selected for a particular property. For example, the library may encode thermostable variants of a particular protein.

Bulk Maturation. In one embodiment, different template nucleic acids are varied within the same reaction mixture. For example, the different template nucleic acids may be different independent hits that are isolated from a primary screen, e.g., the same primary screen or different primary screens. The different templates are hybridized to the same pool of diverse oligonucleotides under controlled hybridization conditions. After diversity strand synthesis, a library can be constructed from the diversity strands. The library includes diversified versions of the each different template nucleic acid.

In one embodiment, the reaction mixture is seeded with the different template nucleic acids such that each template nucleic acid is present at approximately the same concentration as the others. In another embodiment, the templates are seeded at varying concentration. Each template can be seeded as a function of (e.g., in proportion to or in inverse proportion to) its performance in an assay for a function of interest. For example, the library can be constructed such that templates that perform well in an initial screen are included at higher concentration than templates that perform poorly. This approach invests in the diversification of hits that perform well initially. In another example, templates that perform poorly are included at a higher concentration than templates that perform well. This approach is compatible with the hypothesis that hits that perform well initially may be close to their optimum performance, and thus, require less diversification than hits that perform poorly. In still other cases, template sequences are not included in any particular concentration, but are automatically transferred from a pool obtained from an initial screen. For some applications, this may be most expedient.

Primary Screens. The methods described herein can be used to construct a library of nucleic acids for a primary screen. The initial template for the library can be a nucleic acid encoding a particular polypeptide. The particular polypeptide can be a known polypeptide such as a naturally occurring enzyme. In another example, the particular polypeptide is a designed consensus polypeptide, e.g., a consensus immunoglobulin variable domain. The variation method is used to generate variations that differ from the initial template to a controlled extent. For example, the variation method may provide less diversity than the introduction of totally randomized synthetic sequences without hybridization control. However, reduced diversity allows for denser sampling of variants in the region of sequence space defined by the initial template.

Multiplexing Targets. It is possible to screen and mature multiple ligand binding polypeptides that bind to a plurality of different targets in the same reaction mixture. For example, a display library can be screened using an insoluble support that includes more than one different target molecule or using a cell that includes different target molecules on its surface. Members of the library that bind are isolated and varied according to a method described herein, e.g., varied in a single reaction mixture. A secondary library of ligand binding polypeptides is produced from the varied nucleic acids. This secondary library can be rescreened against the same set of complex target molecules or can be deconvolved, e.g., by screening against a subset or individual species of target molecules selected from the original set. In one embodiment, at least two, three, five, or ten different target molecules are screened in multiplex format. Typically, fewer than 30, 20, or 10 different target molecules are used.

Screening Methods

After a library of diversity nucleic acids is constructed, the library can be screened to identify members with a property. For example, the library can be screened to identify members that encode a polypeptide that has an improvement relative to a parental polypeptide encoded by a parental template nucleic acid. However, the library can also be screened to identify members that encode a polypeptide that has impairments relative to the parental polypeptide.

The screening can be performed, e.g., using an assay. The assay can be for a binding property, a catalytic property, a physiological property (e.g., cytotoxicity, renal clearance, immunogenicity), a structural property (e.g., stability, conformation, oligomerization state) or another functional property. The screen can be performed in vitro or in vivo.

Binding properties can be screened using a display library (see below), but also, for example, using a two-hybrid assay, an in vitro binding assay (e.g., using a protein array, or ELISA), or a biological assay (e.g., using cells).

Two-Hybrid Assay. Polypeptides encoded by diversity strands can be tested in a two-hybrid assay or three-hybrid assay to identify variants that bind to a target (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300). The two-hybrid system is based on the modular nature of most transcription factors, which consist of separable DNA-binding and activation domains. Briefly, the assay utilizes two different DNA constructs. In one construct, the gene that codes for a target protein is fused to a gene encoding the DNA binding domain of a known transcription factor (e.g., GAL-4). In the other construct, a varied sequence, e.g., from a library of diversity strands that encodes variants of a parental polypeptide, is fused to a gene that codes for the activation domain of the known transcription factor. (Alternatively the target protein can be fused to the activator domain). If the varied protein and the target protein are able to interact, in vivo, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the gene which encodes the variant protein which interacts with the target protein.

Two-hybrid arrays can be used for library-against-library screens. The variation method can be used generate variants in a first member of binding pair as well as a second member of the binding pair. The first member variants are fused to a transcriptional activation domain, and the second member variants are fused to a DNA binding domain. A matrix is constructed such that each first member variant is combined with each second member variant. Typically, yeast cells that include the respective partners are mated to construct the matrix. Reporter gene activity is monitored in the mated cells to identify combinations for which an interaction is indicated. The method is useful, for example, to redesign protein interaction interfaces and evolve new specificities.

Protein Arrays. Polypeptides encoded by each nucleic acid of a library of diversity strands can be immobilized on a solid support, for example, on a bead or an array. For a protein array, each of the polypeptides is immobilized at a unique address on a support. Typically, the address is a two-dimensional address.

Methods of producing polypeptide arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass.

For example, the array can be an array of antibodies, e.g., as described in De Wildt, supra. A protein array can be contacted with a labeled target to determine the extent of binding of the target to each immobilized polypeptide from the diversity strand library. Information about the extent of binding at each address of the array can be stored as a profile, e.g., in a computer database. The protein array can be produced in replicates and used to compare binding profiles, e.g., of a target and a non-target. Thus, protein arrays can be used to identify individual members of the diversity strand library that have desired binding properties with respect to one or more molecules.

ELISA. Polypeptides encoded by a diversity strand library can also be screened for a binding property using an ELISA assay. For example, each polypeptide is contacted to a microtitre plate whose bottom surface has been coated with the target, e.g., a limiting amount of the target. The plate is washed with buffer to remove non-specifically bound polypeptides. Then the amount of the polypeptide bound to the plate is determined by probing the plate with an antibody that can recognize the polypeptide, e.g., a tag or constant portion of the polypeptide. The antibody is linked to an enzyme such as alkaline phosphatase, which produces a calorimetric product when appropriate substrates are provided. The polypeptide can be purified from cells or assayed in a display library format, e.g., as a fusion to a filamentous bacteriophage coat. In another version of the ELISA assay, each polypeptide of a diversity strand library is used to coat a different well of a microtitre plate. The ELISA then proceeds using a constant target molecule to query each well.

Homogeneous Binding Assays. After a molecule is identified in a fraction, its binding interaction with a target can be analyzed using a homogenous assay, i.e., after all components of the assay are added, additional fluid manipulations are not required. For example, fluorescence energy transfer (FET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FET can be conveniently measured through standard fluorometric detection means well known in the art (e.g., using a fluorimeter). By titrating the amount of the first or second binding molecule, a binding curve can be generated to estimate the equilibrium binding constant.

Surface Plasmon Resonance (SPR). The binding interaction of a molecule isolated from library of diversity strands with a target can be analyzed using SPR. For example, after sequencing of a display library member present in a sample, and optionally verified, e.g., by ELISA, the displayed polypeptide can be produced in quantity and assayed for binding the target using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{d}), and kinetic parameters, including Kₒₙ, and K_{off}, for the binding of a biomolecule to a target. Such data can be used to compare different biomolecules. For example, proteins encoded by nucleic acid selected from a library of diversity strands can be compared to identify individuals that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by crystallography or NMR). As a result, an understanding of the physical interaction between the protein and its target can be formulated and used to guide other design processes.

Cellular Assays. A library of diversity strands can be screened by transforming the library into a host cell. For example, the library can include vector nucleic acid sequences that direct expression of the diversity strands such that polypeptides encoded by the diversity strands are produced, e.g., within the cell or secreted from the cell. If the parental host cell is impaired for a detectable intracellular activity, cells of the library can be identified for which the intracellular activity is restored. For example, the intracellular activity may be a defect in proliferative control, a metabolic activity, or a signaling activity.

In another embodiment, the library of cells is in the form of a cellular array. The cellular array can likewise be screened for any detectable activity.

A molecule in an eluted fraction can be also characterized for a functional activity, e.g., for its ability to affect cell differentiation or cell proliferation in culture (or in vivo or ex vivo). Numerous cell culture assays for differentiation and proliferation are known in the art. Some examples are as follows. Assays for embryonic stem cell differentiation (which will identify, among others, proteins that influence embryonic differentiation hematopoiesis) include, e.g., those described in: Johansson et al. (1995) Cellular Biology 15:141-151; Keller et al. (1993) Molecular and Cellular Biology 13:473-486; McClanahan et al. (1993) Blood 81:2903-2915. Assays for lymphocyte survival/apoptosis (which will identify, among others, proteins that prevent apoptosis after superantigen induction and proteins that regulate lymphocyte homeostasis) include, e.g., those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233 243, 1991; Zacharchuk, Journal of Immunology 145:4037 4045, 1990; Zamai et al., Cytometry 14:891-897, 1993; Gorczyca et al., International Journal of Oncology 1:639-648, 1992. Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. USA 88:7548-7551, 1991.

Exemplary assays for immunological properties are described below.

Automation

In one embodiment, at least some aspects of the variation method are automated. For example, clones isolated from a primary screen are stored in an arrayed format (e.g., microtitre plates). Data indicate the performance of each clone for a particular assay, e.g., a binding assay, an activity assay, or a cell-based assay, can be stored in database. Software can be used to access the database and select clones that meet particular criteria, e.g., exceed a threshold for an assay. The software can then direct a robotic arm to pick the selected clones from the stored array and prepare template nucleic acid from each clone. The robotic arm can further pool the template nucleic acids and dispense the pool in a reaction vessel with a population of diverse oligonucleotides. The reaction vessel can be similarly processed in an automated fashion, e.g., to separate annealed diverse oligonucleotides, form diversity strands, and remove the template nucleic acid strands. Isolated diversity strands can be used to construct a library of diversity strands. Likewise, this library can be screened using automated methods.

These automated methods are suitable for high throughput screening of polypeptides, e.g., synthetic immunoglobulins and other synthetic scaffold-based polypeptides.

In another embodiment, the variation method is performed in a microfluidic system. A microfluidic chip can be etched to include channels that deliver reagents, template nucleic acids, and diverse oligonucleotides to the reaction. Electrokinetic capillary flow can be used to move the reaction components to various regions of the chip (see, e.g., U.S. Pat. No. 6,033,546). The chip can also be controlled to regulate temperature and other factors pertinent for hybridization control. Further, electrokinetic capillary flow can be used to separate annealed from unannealed diverse oligonucleotides.

In one embodiment, the method can further include data analysis and/or other post-variation and post-screening steps. For example, sequences that are identified in a secondary screen of a library constructed using the variation method can be stored in a database, e.g., a computer database. The sequences can be analyzed, e.g., to identify determinants that improve a property of the template nucleic acid for the sequences. The sequences can also be analyzed, e.g., to identify alterations that may be non-beneficial, e.g., deleterious, to the template nucleic acid sequence. Such alterations might be identified in variants that have properties that are less optimal than the template. In another example, variants that are only modestly improved are compared with variants that are substantially improved in order to identify such non-beneficial alterations. The modestly improved variants may include beneficial alterations whose benefit is offset by the non-beneficial alterations.

In some applications, the sequences that encode the improved polypeptides are used to infer the sequence of the template nucleic acid, or the size of the template nucleic acid pool used for variation. Such an analysis can be done using parsimony methods, trees, and clades, e.g., using an assumption for the number of mutations that introduced per template nucleic acid strand under the selected hybridization condition.

Display Libraries

A display library is a collection of entities; each entity includes an accessible polypeptide component and a recoverable component that encodes or identifies the peptide component. The polypeptide component can be of any length, e.g. from three amino acids to over 300 amino acids. A variety of formats can be used for display.

Phage Display. One format utilizes viruses, particularly bacteriophages. This format is termed “phage display.” The varied polypeptide component is typically covalently linked to a bacteriophage coat protein or domain thereof. The linkage can be produced by a translational fusion encoded by a nucleic acid, and joining the varied polypeptide and the invariant bacteriophage coat protein or domain thereof. The linkage can also include a flexible peptide linker, a protease site, or an amino acid incorporated as a result of suppression of a stop codon. Phage display is described, for example, in Ladner et al, U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; WO 94/05781; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982. It is also possible to display multi-chain proteins, e.g., Fabs (see below). Further, the varied polypeptide component can be attached by a non-covalent interaction (e.g., fos-jun dimerization) or a non-peptide covalent bond (e.g., a disulfide linkage).

Phage display systems have been developed for filamentous phage (phage fl, fd, and M13) as well as other bacteriophage (e.g. T7 bacteriophage and lambdoid phages; see, e.g., Santini (1998) J. Mol. Biol. 282:125-135; Rosenberg et al. (1996) Innovations 6:1-6; Houshmand et al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In a preferred embodiment, the fusion is to a domain of the gene III protein, e.g., the anchor domain or “stump,” (see, e.g., U.S. Pat. No. 5,658,727 for a description of the gene III protein anchor domain).

The valency of the peptide component can also be controlled. Cloning of the sequence encoding the peptide component into the complete phage genome results in multivariant display since all replicates of the gene III protein are fused to the peptide component. For reduced valency, a phagemid system can be utilized. In this system, the nucleic acid encoding the peptide component fused to gene III is provided on a plasmid, typically of length less than 700 nucleotides. The plasmid includes a phage origin of replication so that the plasmid is incorporated into bacteriophage particles when bacterial cells bearing the plasmid are infected with helper phage, e.g. M13K01. The helper phage provides an intact copy of gene III and other phage genes required for phage replication and assembly. The helper phage has a defective origin such that the helper phage genome is not efficiently incorporated into phage particles relative to the plasmid that has a wild type origin.

Bacteriophage displaying the peptide component can be grown and harvested using standard phage preparatory methods, e.g. PEG precipitation from growth media.

After selection of individual display phages, the nucleic acid encoding the selected peptide components, by infecting cells using the selected phages. Individual colonies or plaques can be picked, the nucleic acid isolated and sequenced.

Peptide-Nucleic Acid Fusions. Another format utilizes peptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557 and U.S. Provisional Patent Application No. Serial No. 60/326,320, filed Oct. 1, 2001, titled “MULTI-CHAIN EUKARYOTIC DISPLAY VECTORS AND THE USES THEREOF.” This application describes a yeast display system that can be used to display immunoglobulin proteins such as Fab fragments, and the use of mating to generate combinations of heavy and light chains.

In one embodiment, variegate nucleic acid sequences are cloned into a vector for yeast display. The cloning joins the variegated sequence with a domain (or complete) yeast cell surface protein, e.g., Aga2, Aga1, Flo1, or Gas1. A domain of these proteins can anchor the polypeptide encoded by the variegated nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains. Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30 and Schaffitzel et al. (1999) J Immunol Methods. 231(1-2):119-35.

Other Display Formats. Yet another display format is a non-biological display in which the polypeptide component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

Scaffolds. Scaffolds for display can include: antibodies (e.g., Fab fragments, single chain Fv molecules (scFV), single domain antibodies, camelid antibodies, and camelized antibodies); T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains).

Appropriate criteria for evaluating a scaffolding domain can include: (1) amino acid sequence, (2) sequences of several homologous domains, (3) 3-dimensional structure, and/or (4) stability data over a range of pH, temperature, salinity, organic solvent, oxidant concentration. In one embodiment, the scaffolding domain is a small, stable protein domains, e.g., a protein of less than 100, 70, 50, 40 or 30 amino acids. The domain may include one or more disulfide bonds or may chelate a metal, e.g., zinc.

Examples of small scaffolding domains include: Kunitz domains (58 amino acids, 3 disulfide bonds), Cucurbida maxima trypsin inhibitor domains (31 amino acids, 3 disulfide bonds), domains related to guanylin (14 amino acids, 2 disulfide bonds), domains related to heat-stable enterotoxin IA from gram negative bacteria (18 amino acids, 3 disulfide bonds), EGF domains (50 amino acids, 3 disulfide bonds), kringle domains (60 amino acids, 3 disulfide bonds), fungal carbohydrate-binding domains (35 amino acids, 2 disulfide bonds), endothelin domains (18 amino acids, 2 disulfide bonds), and Streptococcal G IgG-binding domain (35 amino acids, no disulfide bonds).

Examples of small intracellular scaffolding domains include SH2, SH3, and EVH domains. Generally, any modular domain, intracellular or extracellular, can be used.

Another useful type of scaffolding domain is the immunoglobulin (Ig) and Ig superfamily domain. Embodiments using Ig domains for display are described below (see, e.g., “Antibody Maturation”).

Display technology can also be used to obtain ligands, e.g., antibody ligands, particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

Iterative Selection. In one preferred embodiment, display library technology is used in an iterative mode. A first display library is used to identify one or more ligands for a target. These identified ligands are then varied using a method described herein to form a second display library. Higher affinity ligands are then selected from the second library, e.g., by using higher stringency or more competitive binding and washing conditions.

If, for example, the identified ligands are antibodies, then mutagenesis can be directed to the CDR regions of the heavy or light chains as described herein. Further, mutagenesis can be directed to framework regions near or adjacent to the CDRs. Likewise, if the identified ligands are enzymes, mutagenesis can be directed to the active site.

Off-Rate Selection. Since a slow dissociation rate can be predictive of high affinity, particularly with respect to interactions between polypeptides and their targets, the methods described herein can be used to isolate variants with an improved kinetic dissociation rate (i.e. reduced) for a binding interaction to a target relative to the kinetic dissociation rate of the initial molecule.

To select for such variants from a display library that displays diversity strands, the library is contacted to an immobilized target. The immobilized target is then washed with a first solution that removes non-specifically or weakly bound biomolecules. Then the immobilized target is eluted with a second solution that includes a saturation amount of free target, i.e., replicates of the target that are not attached to the particle. The free target binds to biomolecules that dissociate from the target. Rebinding is effectively prevented by the saturating amount of free target relative to the much lower concentration of immobilized target.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include biomolecules that dissociate at a slower rate from the target than biomolecules in the early fractions.

Antibody Maturation

In one embodiment, the method is used to generate variants in an immunoglobulin domain, e.g., an immunoglobulin variable domain.

An “immunoglobulin domain” refers to a domain of immunoglobulin molecules, e.g., a variable or constant domain. An “immunoglobulin superfamily domain” refers to a domain that has a three-dimensional structure related to an immunoglobulin domain, but is from a non-immunoglobulin molecule. Immunoglobulin domains and immunoglobulin superfamily domains typically include two β-sheets formed of about seven β-strands, and a conserved disulphide bond (see, e.g., A. F. Williams and A. N. Barclay 1988 Ann. Rev Immunol. 6:381-405). Proteins that include domains of the Ig superfamily domains include T cell receptors, CD4, platelet derived growth factor receptor (PDGFR), and intercellular adhesion molecule (ICAM).

A preferred embodiment of immunoglobulin scaffolds is an antibody, particularly an antigen-binding fragment of an antibody. The term “antibody,” as used herein, refers to an immunoglobulin molecule or an antigen-binding portion thereof. A typical antibody includes two heavy (H) chain variable regions (abbreviated herein as VH), and two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extents of the framework region and CDRs have been precisely defined (see, Kabat, E. A., et al. (1991) Sequences of Proteins of immunological Interest, Fifth Edition, U.S. Department of Health and Human Services, NIH Publication No. 91-3242, and Chothia, C. et al. (1987) J. Mol. Biol. 196:901-917). The Kabat definitions are controlling herein. Each VH and VL is composed of three CDR's and four FRs, arranged from amino-terminus to carboxy-terminus in the following order: FR1, CDR1, FR2, CDR2, FR3, CDR3, FR4.

An antibody can also include a constant region as part of a light or heavy chain. Light chains can include a kappa or lambda constant region gene at the COOH—terminus. Heavy chains can include, for example, a gamma constant region (IgG1, IgG2, IgG3, IgG4; encoding about 330 amino acids).

The term “antigen-binding fragment” of an antibody (or simply “antibody portion,” or “fragment”), as used herein, refers to one or more fragments of a full-length antibody that retain the-ability to specifically bind to a target. Examples of antigen-binding fragments include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody.

The variation method described herein can be used to introduce diversity into any immunoglobulin domain, for example. The subject immunoglobulin domain typically has at least a minimal binding specificity for a target or a minimal activity, e.g., an equilibrium dissociation constant for binding of greater than 10 nM, 100 nM, or 1 μM. However, an immunoglobulin domain can also be naïve, e.g., a consensus immunoglobulin domain, which is not selected for a particular activity.

The nucleic acid sequence encoding a particular immunoglobulin domain is used as a template nucleic acid that functions to receive the introduced diversity. The nucleic acid can include other sequences, e.g., such that an antibody chain (e.g., a heavy or light chain) is encoded, or such that two antibody chains are encoded (e.g., a heavy and a light chain in the format of a Fab or a full-length antibody).

The single-stranded template nucleic acid can be obtained from a cloned nucleic acid sequence or from an un-cloned sequence. For example, the single-stranded template nucleic acid can be obtained as a single-stranded plasmid, e.g., a phage genome or a phagemid. The phage genome or phagemid includes a sequence that encodes the particular immunoglobulin domain. In another example, the single-stranded template is obtained from an amplification reaction, e.g., a PCR reaction. One of the PCR primers can be tagged so that one of the two amplified strands from the PCR reaction can be captured, e.g., using a solid support that recognizes the tag.

After, the template is rendered single-stranded, it is annealed to diverse oligonucleotides. Methods for providing diverse oligonucleotides for immunoglobulin domains are described below (see “Repertoire for Immunoglobulin Diversity”).

The method can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible.

Further, the method can equally be applied to introduce variation into different template nucleic acids, i.e., to vary different subject immunoglobulins. Such variation can be performed in parallel, e.g., in the same reaction vessel or independently. The multiple immunoglobulin domains can be unrelated in sequence (e.g., having differing hypervariable regions) or in property, although typically such domains are related by having a minimal specificity for a common target compound. For example, different antibodies obtained in a first display selection for a target compound can be matured in parallel.

In particular, the method can include a hybridization step that is tuned according to the amount of diversity required. Hybridization conditions can be varied. As discussed elsewhere herein, low stringency provides for many possible significant variations, e.g., variations which might improve an immunoglobulin domain having low affinity for the target into one having high affinity for the target. High stringency provides for fewer significant variations, e.g., while maintaining many features of the template yet introducing a few variations that might improve an immunoglobulin domain having a high affinity for a target into one with even higher affinity. If different regions are varied, for example, the hybridization conditions can be different for each region or the diverse oligonucleotides can be designed so that they are compatible for use-under the same conditions.

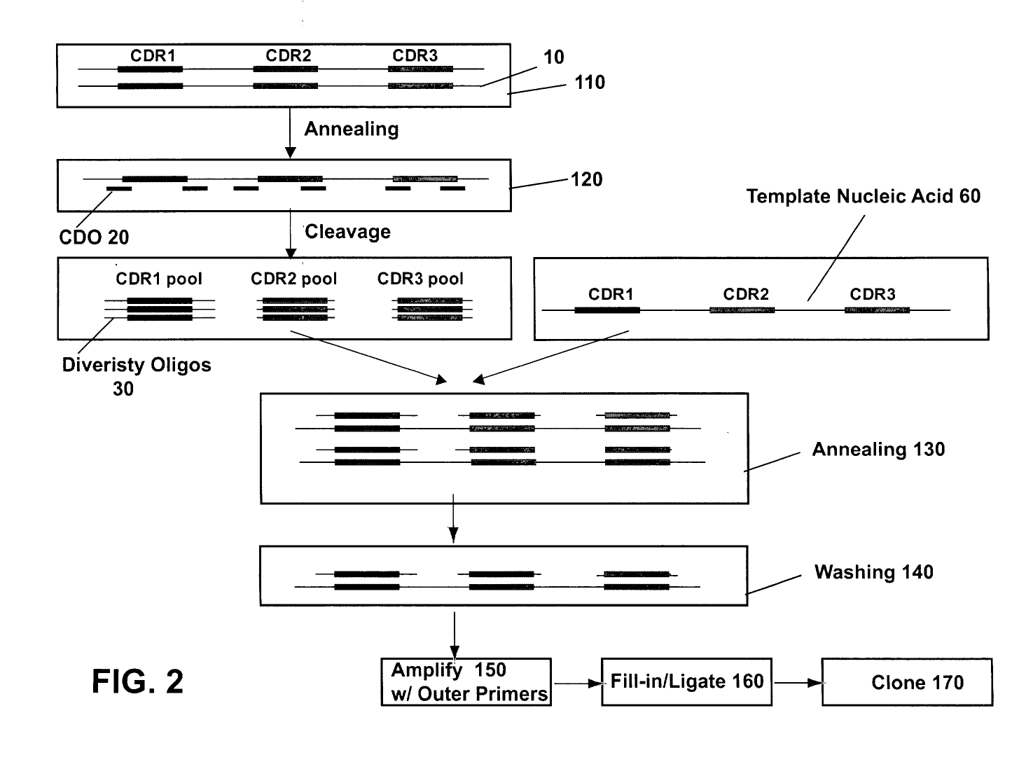

Referring now to FIG. 2, one exemplary antibody variation process is set forth. A pool of diverse nucleic acids (labeled “10” in FIG. 2), e.g., immunoglobulin cDNA from B cells, is provided. The pool of cDNAs encodes a diverse number of immunoglobulin variable domains. Cleavage-directing oligonucleotides (labeled “CDO 20” in FIG. 2) are annealed and used to excise diverse oligonucleotides (labeled “30” in FIG. 2). The method for these two steps is described in FIG. 1 and below. Three pools of diverse oligonucleotides 30, one pool for each CDR, are prepared either separately or from the same cDNA. Each diverse oligonucleotide includes a sequence encoding a single CDR and a portion of the flanking framework region, or a complement of such sequence.

The template nucleic acid (labeled “60” in FIG. 2) which encodes a variable domain is combined with one or more of the three pools of CDR diverse oligonucleotides. The diverse oligonucleotides are annealed to the template nucleic acid (see, e.g., block 130 of FIG. 2). The template nucleic acids are washed (see, e.g., block 140 of FIG. 2) to remove weakly bound diverse oligonucleotides. The washing conditions can be more stringent than the annealing conditions, i.e., the “tuned” hybridization conditions can be implemented during the washing phase of the hybridization reaction rather than (or in addition to) the initial annealing. The annealed oligonucleotides are then filled-in and ligated. The diversity strand can be amplified, e.g., using an outer primer. The amplified diversity strand can then be cloned. This process can be performed on multiple template nucleic acid strands, e.g., many replicates of one or more template nucleic acids, to form a library of diversified strands, e.g., a display library or an expression library.

Immunoglobulin domains can be displayed in a variety of formats. One format is the single chain Fv format (scFv). As generally described in McCafferty et al., Nature (1990) 348:552-554, scFv polypeptides include the complete VH and VL domains of an antibody joined by a flexible (Gly₄-Ser₃) linker. Such domains can have demonstrable antigen affinity. Some scFv's can form higher molecular weight species including dimers (Weidner et al. (1992) J. Biol. Chem. 267:10281-10288; Holliger et al. (1993) Proc. Natl. Acad. Sci. U.S.A. 90, 6444-6448) and trimers (Kortt et al., (1997) Protein Eng. 10: 423-433), which can complicate both selection-and characterization.

Another format is the Fab format. The Fab display format in which a variable domain from a heavy or light chain gene is linked to a phage coat protein domain (e.g., a minor coat protein domain, e.g., the gene III anchor domain or “stump”), and in some embodiments, also carries a tag for detection and purification. The other chain is expressed as a separate fragment which is secreted into the periplasm, where it can pair with the first chain that is fused to the phage coat protein (Hoogenboom, et al., (1991) Nucleic Acids Res. 19:4133-4137). In one embodiment, the variable domain from a heavy chain gene is fused to the phage coat protein and the light chain gene is expressed as a separate fragment. A Fab can also be displayed on the surface of a eukaryotic cell, e.g., a yeast cell. The Fab can be connected to a yeast cell surface protein.

The choice for the Fab format was based on the notion that the monomeric display of the Fab permits the rapid screening of large numbers of clones for kinetics of binding (off-rate) with crude protein fractions. This reduces the time for post-selection analysis dramatically when compared to that needed for selected single-chain Fv (scFv) antibodies from phagemid libraries (Vaughan, et al., (1996) Nat. Biotechnol. 14, 309-314; Sheets, et al., (1998) Proc. Natl. Acad. Sci. U.S.A. 95:6157-6162), or Fab fragments from other phage libraries (Griffiths, et al., (1993) EMBO J. 12:725-734).

Antibody Production. Some antibodies, e.g., Fabs, can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be shuffled into a bacterial cell that cannot suppress a stop codon. In this case, the Fab is not fused to the gene III protein and is secreted into the media.

Antibodies can also be produced in eukaryotic cells. In one embodiment, the antibodies (e.g., scFv's) are expressed in a yeast cell such as Pichia (see, e.g., Powers et al. (2001) J Immunol Methods. 251:123-35).

In one preferred embodiment, antibodies are produced in mammalian cells. Preferred mammalian host cells for expressing the clone antibodies or antigen-binding fragments thereof include Chinese Hamster Ovary (CHO cells) (including dhfr− CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, and a cell from a transgenic animal, e.g., a transgenic mammal. For example, the cell is a mammary epithelial cell.

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻host cells with methotrexate selection/amplification) and the neo gene (for G418 selection).

In an exemplary system for recombinant expression of a modified antibody, or antigen-binding portion thereof, a recombinant expression vector encoding both the antibody heavy chain and the antibody light chain is introduced into dhfr− CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G.

Antibody Assays. Antibody variants identified from phage display can be screened, e.g., for a binding property using ELISA or SPR. The antibody variants can also be purified, e.g., from a mammalian cell and used in a functional assay, e.g., an assay for complement activation and killing of a cell expressing the antigen or an assay for antibody-dependent cell-mediated cytotoxicity.

The methods described herein can be used to vary human or “humanized” antibodies, particularly those that recognize human antigens. Such antibodies can be used as therapeutics to treat human disorders such as cancer. Since the constant and framework regions of the antibody are human, these therapeutic antibodies may avoid themselves being recognized and targeted as antigens. The constant regions are also optimized to recruit effector functions of the human immune system. The in vitro “maturation” process also bypasses the inability of a normal human immune system to generate antibodies against self-antigens.

Repertoire for Immunoglobulin Diversity

The diverse oligonucleotides that are used to vary an immunoglobulin domain can be obtained from a variety of synthetic or natural sources.

In a preferred embodiment, immune cells can be used as a natural source of diversity for the variation of antibodies, MHC-complexes and T cell receptors. Some examples of immune cells are B cells and T cells. The immune cells can be obtained from, e.g., a human, a primate, mouse, rabbit, camel, or rodent. In a preferred embodiment, the cells are selected for a particular property. For example, T cells that are CD4⁺ and CD8⁻ can be selected. B cells at various stages of maturity can be selected.

In one preferred embodiment, fluorescent-activated cell sorting is used to sort B cells that express surface-bound IgM, IgD, or IgG molecules. Further B cells expressing different isotypes of IgG can be isolated. In another preferred embodiment, the B or T cell is cultured in vitro. The cells can be stimulated in vitro, e.g., by culturing with feeder cells or by adding mitogens or other modulatory reagents, such as antibodies to CD40, CD40 ligand or CD20, phorbol myristate acetate, bacterial lipopolysaccharide, concanavalin A, phytohemagglutinin or pokeweed mitogen.

In still another preferred embodiment, the cells are isolated from a subject that has an immunological disorder, e.g., systemic lupus erythematosus (SLE), rheumatoid arthritis, vasculitis, Sjogren syndrome, systemic sclerosis, or anti-phospholipid syndrome. The subject can be a human, or an animal, e.g., an animal model for the human disease, or an animal having an analogous disorder. In still another embodiment, the cells are isolated from a transgenic non-human animal that includes a human immunoglobulin locus.

In one preferred embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibodies (see, e.g., Bergthorsdottir et al. (2001) J Immunol. 166:2228). In another embodiment, the cells are naïve.

Diverse oligonucleotides from a natural repertoires can be obtained, for example from genomic DNA or mRNA is isolated from the afore-mentioned cells.

In a preferred embodiment, the cDNA is produced from the mRNAs using reverse transcription. For example, RNA is isolated from the cell. Full length (i.e., capped) mRNAs are separated (e.g. by degrading uncapped RNAs with calf intestinal phosphatase). The cap is then removed with tobacco acid pyrophosphatase and reverse transcription is used to produce the cDNAs.

The reverse transcription of the first (antisense) strand can be done in any manner with any suitable primer. See, e.g., de Haard et al. (1999) J. Biol. Chem 274:18218-30. The primer binding region can be constant among different immunoglobulins, e.g., in order to reverse transcribe different isotypes of immunoglobulin. The primer binding region can also be specific to a particular isotype of immunoglobulin. Typically, the primer is specific for a region that is 3′ to a sequence encoding at least one CDR.

In another embodiment, poly-dT primers may be used (and may be preferred for the heavy-chain genes).

In one embodiment, a synthetic sequence is ligated to the 3′ end of the reverse transcribed strand. The synthetic sequence can be used as a primer binding site for amplification after reverse transcription.

In one preferred embodiment, the reverse transcriptase primer or an amplification primer may be biotinylated, thus allowing the cDNA product to be immobilized on streptavidin (Sv) beads. Immobilization can also be effected using a primer labeled at the 5′ end with one of a) free amine group, b) thiol, c) carboxylic acid, or d) another group not found in DNA that can react to form a strong bond to a known partner on an insoluble medium. If, for example, a free amine (preferably primary amine) is provided at the 5′ end of a DNA primer, this amine can be reacted with carboxylic acid groups on a polymer bead using standard amide-forming chemistry.

After reverse transcription and/or amplification, the top strand RNA can be degraded using well-known enzymes, such as a combination of RNAseH and RNAseA, either before or after immobilization of complementary DNA strands. In another embodiment, an adaptor is ligated to a cDNA strand. The adaptor includes a tag for immobilizing the ligated strand to a solid support.

The diversity nucleic acid can be amplified before being used as a source of diverse oligonucleotides. Any method for amplifying nucleic acid sequences may be used for such amplification. Methods that maximize, and do not bias, diversity are preferred. A variety of techniques can be used for nucleic acid amplification. The polymerase chain reaction (PCR; U.S. Pat. Nos. 4,683,195 and 4,683,202, Saiki, et al. (1985) Science 230, 1350-1354) utilizes cycles of varying temperature to drive rounds of nucleic acid synthesis. Transcription-based methods utilize RNA synthesis by RNA polymerases to amplify nucleic acid (U.S. Pat. No. 6,066,457; U.S. Pat. No. 6,132,997; U.S. Pat. No. 5,716,785; Sarkar et. al., Science (1989) 244: 331-34; Stofler et al., Science (1988) 239: 491). NASBA (U.S. Pat. Nos. 5,130,238; 5,409,818; and 5,554,517) utilizes cycles of transcription, reverse-transcription, and RnaseH-based degradation to amplify a DNA sample. Still other amplification methods include rolling circle amplification (RCA; U.S. Pat. Nos. 5,854,033 and 6,143,495) and strand displacement amplification (SDA; U.S. Pat. Nos. 5,455,166 and 5,624,825).

The amplified nucleic acids can either be used directly to prepare diverse oligonucleotides, or can be stored, e.g., by cloning them into a vector and preparing a library that can serve as a source of diversity

To use the amplified nucleic acid directly as a source of diversity, the amplified nucleic acids are rendered single-stranded. For example, the strands can be separated by using a biotinylated primer, capturing the biotinylated product on streptavidin beads, denaturing the DNA, and washing away the complementary strand. Depending on which end of the captured DNA is wanted, one will choose to immobilize either the upper (sense) strand or the lower (antisense) strand. In another example, the upper strand or lower strand primer may be also biotinylated or otherwise tagged at the 5′ end with one of a) free amino group, b) thiol, c) carboxylic acid and d) another group not found in DNA that can react to form a strong bond to a known partner as an insoluble medium. These can then be used to immobilize and then isolate the tagged strand (formed by extension of the tagged primer) after amplification.

In one embodiment, the amplified single-stranded diversity nucleic acids are then cleaved in order to produce diverse oligonucleotides. Cleavage can be mediated by oligonucleotides, e.g., as described above and in the Examples.

Design of the cleavage-directing oligonucleotides for either method can be done using computer software that analyzes nucleic acid sequence encoding immunoglobulin genes, e.g., germline nucleic acid sequences from a catalog of germline sequences. See, e.g., on-line resources regarding antibody germline sequences provided by the Medical Research Council, Cambridge, UK, as can be located by a standard Internet search service such as GOOGLE™. For other families, similar comparisons exist and may be used to select appropriate regions for cleavage and to maintain diversity. Likewise, such sequences can be obtained from GenBank® at the National Center for Biotechnology Information (Bethesda Md.).

Cleavage-directing oligonucleotides are designed to excise nucleic acid, for example from nucleic acid encoding a CDR region. Optional criteria for the cleavage-directing oligonucleotides include one or more of: a) short length, e.g., between 12 and 24 nucleotides; b) location adjacent to, but not overlapping with hot spots of germline or somatic diversity; c) complementarity to >95% of nucleic acids encoding the Ig domain of interest; and d) availability and location of restriction enzyme recognition sites. Preferably, the available restriction enzyme recognition sites are for restriction enzymes that can cleave nucleic acid at a temperature above 40° C. or above 50° C. Ideally, the restriction enzymes are highly specific, for example, they do not cut single-stranded nucleic acid or short hairpins or heteroduplexes that might form from secondary structure present in an otherwise single-stranded template nucleic acid.

One design consideration is the identification of enzymes which cleave as many related members of a diverse pool of nucleic acid sequences as possible. Further, the method can include providing a cocktail of cleavage oligonucleotides and a cocktail of restriction enzymes in order to cleave different immunoglobulin chain family members in a single reaction. In another example, nucleic acids encoding different family members are provided separately in pools, and each pool is contacted with the cleavage oligonucleotide that anneals to the majority of the family, and is cleaved with the restriction enzyme that is useful for that family.

In a preferred embodiment, the nucleic acids of the repertoire are attached to a solid support and are cleaved sequentially according to the method described in Example 3. In another embodiment, the cleavage-directing oligonucleotides include a recognition site for a Type IIS restriction enzyme, e.g., as described above. Such a design obviates the need for a palindromic site or other site recognized by Type II restriction site.

Diverse oligonucleotides are collected from the digestion of the diverse nucleic acids and used, for example, in variation-introducing method described above.

Peptide and Scaffold Domain Variation

In one embodiment, a nucleic acid variation method described herein is used to vary a nucleic acid encoding a peptide, e.g., a peptide ligand that specifically binds to a target or, generally, to vary a nucleic acid encoding any proteinaceous domain, e.g., a domain that binds to a target or participates in binding to a target. The peptide ligand or other target-binding ligand be identified using a display library, e.g., as described below.

Synthetic Peptides. The binding ligand can include an artificial peptide of 32 amino acids or less, that independently binds to a target molecule. Some synthetic peptides can include one or more disulfide bonds. Other synthetic peptides, so-called “linear peptides,” are devoid of cysteines. Synthetic peptides may have little or no structure in solution (e.g., unstructured), heterogeneous structures (e.g., alternative conformations or “loosely structured), or a singular native structure (e.g., cooperatively folded). Some synthetic peptides adopt a particular structure when bound to a target molecule. Some exemplary synthetic peptides are so-called “cyclic peptides” that have at least disulfide bond, and, for example, a loop of about 4 to 12 non-cysteine residues. Many exemplary peptides are less than 28, 24, 20, or 18 amino acids in length.

Peptide sequences that independently bind a molecular target can be selected from a display library or an array of peptides. After identification, such peptides can be produced synthetically or by recombinant means. The sequences can be incorporated (e.g., inserted, appended, or attached) into longer sequences.

An exemplary phage display displays a short, variegated exogenous peptide on the surface of M13 phage. The peptide display library can be synthesized from synthetic oligonucleotides that are designed to have between 4 and 30 varied codon positions, e.g., a segment of 4, 5, 6, 7, 8, 10, 11, or 12 varied codons, flanked by codons for cysteine residues (or complement thereof). The pairs of cysteines are believed to form stable disulfide bonds, yielding a cyclic display peptide. The oligonucleotides can be cloned into a format suitable for display, e.g., so that the varied peptides are displayed at the amino terminus of protein III on the surface of the phage. For example, to produce a loop of four amino acids in a 12 amino acid long sequence, a library is constructed using a template sequence that includes three varied codon positions, a codon encoding cysteine, four varied codon positions, a codon encoding cysteine, and three varied codon positions. The varied codon positions can include a codon encoding any amino acid except cysteine. Such variation can be generated using trinucleotide subunits for nucleic acid synthesis. The patterning and extent of variation can also be precisely controlled, e.g., to generate loops of other sizes and compositions. Cysteine can be omitted altogether to prepare linear peptides. For example, the Lin20 library was constructed to display a single linear peptide in a 20-amino acid template. The amino acids at each position in the template were varied to permit any amino acid except cysteine (Cys).

The techniques discussed in Kay et al., Phage Display of Peptides and Proteins: A Laboratory Manual (Academic Press, Inc., San Diego 1996) and U.S. Pat. No. 5,223,409 are useful for preparing a library of potential binders corresponding to the selected parental template. The libraries described above can be prepared according to such techniques, and screened, e.g., as described above, for peptides that bind to a particular molecular target.

After one or more peptides are selected, template nucleic acids encoding the one or more peptides (or complements thereof) can be prepared. These peptides can be varied in a controlled manner by annealing a diverse set of oligonucleotides, e.g., the oligonucleotides used to construct the original library, under conditions such that only a subset of the oligonucleotides bind. The hybridization conditions favor the annealing oligonucleotides that encode a sequence that has some similarity to the template nucleic acid, so that at least some codons are retained from the originally selected peptides. Diversified nucleic acids that incorporate the annealed oligonucleotides are synthesized to prepare a secondary display library of peptides. In some implementations (e.g., for peptides less than 12 amino acids), it may not be necessary to extend these oligonucleotides, but merely to ligate them to a nucleic acid encoding an invariant sequence (e.g., the anchor protein). Thus, in these implementations, copying of the template strand is not required. For example the oligonucleotide mixture may be retrieved by denaturation of the oligonucleotide-template hybrids and directly cloned on the basis of complementary regions bordering the area of diversity, or after PCR of the retained oligonucleotides. Alternatively the mutant strands are rescued via a Kunkel mutagenesis procedure as described earlier.

An advantage of such mutagenesis procedure is that it is not necessary to characterize the sequences of individual clones, but that whole collections of selected populations can be mutagenized, even without understanding the genetic complexity of the selected population. Thus in one application the prior identification of a consensus sequence is not required. This approach will allow the affinity selection of clones that do not follow a particular consensus as defined after the first round of selection/screening/analysis, and are rare in the initially selected population; often frequency and consensus considerations are used to delete such clones for further analysis or maturation. When this strategy of mutagenesis by hybridization is applied for multiple rounds and carried out under increasing stringency (e.g., one or more of: increased stringency hybridization conditions, thereby gradually reducing the number of mutations introduced; and increased stringency selection, e.g. gradually increasing the stringency of washing when selection for binding to antigen), it is expected that the initial peptide or protein sequence is iteratively matured. The focused access of sequence space can be particularly useful.

Other Exemplary Scaffolds. Other exemplary scaffolds that can be variegated to produce a protein that binds to serum albumin and a particular target can include: extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (including inactivated proteases), RNase; chaperones, e.g., thioredoxin, and heat shock proteins; and intracellular signaling domains (such as SH2 and SH3 domains) and antibodies (e.g., Fab fragments, single chain Fv molecules (scFV), single domain antibodies, camelid antibodies, and camelized antibodies); T-cell receptors and MHC proteins. In many embodiments, the scaffold may be less than 50 amino acids in length.

U.S. Pat. No. 5,223,409 also describes a number of so-called “mini-proteins,” e.g., mini-proteins modeled after α-conotoxins (including variants GI, GII, and MI), mu-(GIIIA, GIIIB, GIIIC) or OMEGA-(GVIA, GVIB, GVIC, GVIIA, GVIIB, MVIIA, MVIIB, etc.) conotoxins. U.S. Pat. No. 6,423,498 describes an exemplary library of varied Kunitz domains and methods for constructing such a library.

As described above for peptide and immunoglobulin domains, after a domain is selected for a particular property, a template nucleic acid encoding it (and optionally other such domains) can be prepared and then varied by annealing diverse oligonucleotides, e.g., synthetic oligonucleotides or oligonucleotides derived from a natural source. The hybridization conditions are controlled to favor the annealing oligonucleotides that encode a sequence that has some similarity to the template nucleic acid, so that at least some codons are retained from the originally selected domains. A secondary display library can then be prepared and screened.

Targets for Ligand Design

The method can be used to generate variants in a polypeptide in order to identify a variant that binds a target, particularly to identify an improved variant that binds a target. Some exemplary targets include: cell surface proteins (e.g., glycosylated surface proteins or hypoglycosylated variants), cancer-associated proteins, cytokines, chemokines, peptide hormones, neurotransmitters, cell surface receptors (e.g., cell surface receptor kinases, seven transmembrane receptors, virus receptors and co-receptors, extracellular matrix binding proteins, cell-binding proteins, antigens of pathogens (e.g., bacterial antigens, malarial antigens, and so forth).

More specific examples include: integrins, cell attachment molecules or “CAMs” such as cadherins, selections, N-CAM, E-CAM, U-CAM, I-CAM and so forth); proteases, e.g., subtilisin, trypsin, chymotrypsin; a plasminogen activator, such as urokinase or human tissue-type plasminogen activator (t-PA); bombesin; factor IX, thrombin; CD-4; platelet-derived growth factor; insulin-like growth factor-I and -II; nerve growth factor; fibroblast growth factor (e.g., aFGF and bFGF); epidermal growth factor (EGF); transforming growth factor (TGF, e.g., TGF-α and TGF-β); insulin-like growth factor binding proteins; erythropoietin; thrombopoietin; mucins; human serum albumin; growth hormone (e.g., human growth hormone); proinsulin, insulin A-chain insulin B-chain; parathyroid hormone; thyroid stimulating hormone; thyroxine; follicle stimulating hormone; calcitonin; atrial natriuretic peptides A, B or C; leutinizing hormone; glucagon; factor VIII; hemopoietic growth factor; tumor necrosis factor (e.g., TNF-α and TNF-β); enkephalinase; Mullerian-inhibiting substance; gonadotropin-associated peptide; tissue factor protein; inhibin; activin; vascular endothelial growth factor; receptors for hormones or growth factors; protein A or D; rheumatoid factors; osteoinductive factors; an interferon, e.g., interferon-α,β,γ; colony stimulating factors (CSFs), e.g., M-CSF, GM-CSF, and G-CSF; interleukins (ILs), e.g., IL-1, IL-2, IL-3, IL-4, etc.; decay accelerating factor; immunoglobulin (constant or variable domains); and fragments of any of the above-listed polypeptides. In some embodiments, the target is associated with a disease, e.g., cancer.

Additional targets for binding interactions include transition state intermediates. For example, polypeptides can be selected that bind and stabilize transition state intermediates. Frequently, such polypeptides can catalyze a chemical reaction that proceeds through the intermediate. The polypeptide being varied can be totally synthetic, or based upon a natural scaffold, e.g., an antibody or an enzyme such as, proteases (blood-clotting proteases), enolases, cytochrome P450s, acyltransferases, methylases, TIM barrel enzymes, isomerases, acyl transferases, and so forth.

Non-Coding Nucleic Acid Variation

The method can also be used to introduce variation into nucleic acid sequences that do not encode polypeptides. Examples of such nucleic acid sequences include regulatory sequences (e.g., transcriptional, translational, and chromosomal regulatory sequences), ribozymes, and functional synthetic nucleic acids.

Nucleic acids that are artificial ligands and catalysts, so-called “nucleic acid aptamers” can be isolated from random pools of nucleic acid sequences (see, e.g., Ellington and Szostak (1990) Nature 346:818; and (1992) Nature 355:850; and Tuerk and Gold ((1990) Science 249:505 and (1991) J. Mol. Biol. 222:739; U.S. Pat. No. 5,910,408). Both RNA and DNA can have such binding and/or catalytic functions. The variation method described herein can be used to modify a template nucleic acid that has at least a threshold binding or catalytic activity, or its complement.

The following examples are merely illustrative of particular aspects of the invention described herein.

### EXAMPLES

### Example 1

### Design of Oligonucleotides for Cleavage of the PH1 Immunoglobulin Gene

PH1 is a human antibody directed to human MUC1, isolated from a phage antibody library (see US Published Application 2002/0146750, filed Mar. 30, 2001). Restriction enzyme sites were identified to excise CDR1 from the nucleic acid that encodes part of a signal sequence and the PH1 kappa light chain. This nucleic acid sequence and its translation are listed as follows.

| | | |
|---|---|---|
| 1 | TCTCACAGTGCACTTGAAATTGTGCTGACTCAGTCTCCACTCTCCCTGCCCGTCACCCCT | (SEQ ID NO:1) |
| 1 | S H S A L E I V L T Q S P L S L P V T P | (SEQ ID NO:2) |
| | CDR1 | |
| | ------------------------------------ | |
| 61 | GGAGAGCCGGCCTCCATCTCCTGCAGGTCTAGTCAGAGCCTCCTGCATAGTAATGGATAC | |
| 21 | G E P A S I S C R S S Q S L L H S N G Y | |
| | ----------- | |
| 121 | ACCTATTTGGATTGGTACCTCCAGAAGCCAGGGCAGTCTCCACAGCTCCTGATCTATTCG | |
| 41 | T Y L D W Y L Q K P G Q S P Q L L I Y S | |
| 181 | GGTTCTCATCGGGCCTCCGGGGTCCCTGACAGGTTCAGTGGCAGTGTATCAGGCACAGAT | |
| 61 | G S H R A S G V P D R F S G S V S G T D | |
| 241 | TTTACACTGAGAATCAGCAGAGTGGAGGCTGAGGATGTTGGAGTTTATTACTGCATGCAG | |
| 81 | F T L R I S R V E A E D V G V Y Y C M Q | |
| 301 | GGTCTACAGAGTCCATTCACTTTCGGCCCTGGGACCAAAGTGGATATCAAACGAGGAACT | |
| 101 | G L Q S P F T F G P G T K V D I K R G T | |
| 361 | GTGGCTGCACCATCTGTCTTCATCTTCCCGCCA | |
| 121 | V A A P S V F I F P P | |

The position of CDR1 is indicated above. Four regions of the nucleic acid sequence were analyzed for the presence of restriction enzyme site sequences from a database of 180 restriction enzyme site sequences. Table 2 lists the four regions, the sequence of each region, the restriction enzyme (RE) site identified, and the length of the overhang within the region. Most enzymes have optimal function at 37° C., but retain some activity at higher temperatures; other enzymes have optimal function at higher temperature. For example, BstNI has its optimal function at 60° C.

TABLE 2
Examples of Region to which Cleavage-Directing Oligonucleotides
may anneal and direct cleavage (for VκII family)

| | | | Nucl. |
|---|---|---|---|
| | | | Left |
| | | | after |
| Region | Sequence of Region | RE | RE |
| 3′ FR1 | GGAGAGCCGGCCTCCATCTCCTGC | Cfr10I | 18/14 |
| | (SEQLDNO:3) | Eco56I | 18/14 |
| | | HaeIII | 14/14 |
| | | HpaII | 17/15 |
| | | Mn1I | 3/4 |
| | | MspI | 17/15 |
| | | NaeI | 16/16 |
| 3′ FR1 & | ATCTCCTGCAGGTCTAGTCAGAGC | MaeI | 14/17 |
| 5′ CDR1 | (SEQ ID NO:4) | PstI | −1/3 |
| 5′ FR2 | TGGTACCTGCAGAAGCCAGGGCAG | BspMI | 14/18 |
| | (SEQ ID NO:5) | BstNI | 17/18 |
| | | EcoRII | 15/20 |
| | | ScrFI | 17/18 |
| 5′ FR2 | TGGTACCTGCAGAAGCCAGGGCAG | Acc65I | 2/6 |
| and | (SEQ ID NO:5) and | Asp718I | 2/6 |
| 5′ FR2 & | TATTTGGATTGGTACCTGCAGAAG | BanI | 2/6 |
| 3′ CDR1 | (SEQ ID NO:6) | KpnI | 6/2 |
| | | NlaIV | 4/4 |
| | | PstI | 11/7 |
| | | RsaI | 4/4 |

RE = Restriction Enzyme.
“Nucl. Left after RE” = the number of nucleotides remaining on the top and bottom strands after restriction.

The restriction enzymes listed above can be used to cleave the nucleic acid sequence of PH1 in a double stranded region formed by a cleavage-directing oligonucleotide to release an oligonucleotide encoding CDR1. For example, a cleavage directing oligonucleotide complementary to SEQ ID NO:3 can be annealed to a template nucleic acid encoding a VκII domain and cleaved with MnlI; and a cleavage directing oligonucleotide complementary to SEQ ID NO:5 can be annealed to the template and then cleaved with Asp718I to release an oligonucleotide encoding CDR1. When applied to a diverse population of VκII domain-encoding nucleic acids, diverse CDR1-encoding oligonucleotides are produced.

### Example 2

### Design of Oligonucleotides for Cleavage of Human Light Chain Immunoglobulin Genes

The analysis strategy described for PH1 was applied to a repertoire of genes encoding human immunoglobulin light chains. Families of these genes were scanned for the presence of useful restriction sites in the regions 3′ FR1, 3′FR1 & 5′CDR1, 5′FR2 and 5′FR2 & 3′ CDR1. The results are based on selected sequences from a database of 112 Vκ and 118 Vλ sequences and frequency information from de Wildt et al. (2000) Eur. J. Immunol. 30:254-261. Table 3 summarizes the number of enzymes identified for each isotype. In combination with an appropriate cleavage-directing oligonucleotide, the identified enzymes can be used to excise diverse oligonucleotides that encode light chain CDR1 from most immunoglobulin genes of the listed isotypes. Among the enzymes is BstNI which has its optimal function around 60° C.

Analysis of nucleic acid sequences encoding each of the predominant Vκ and Vλ light chain has led to the identification of restriction enzymes that are suitable for excision of diverse oligonucleotides from the sequences. The number of identified enzymes for each isotype is summarized as follows:

TABLE 3
Number of Identified Restriction Enzyme Sites for
VL CDR1 Nucleic Acid Excision

| Family | 5′ of CDR1 | 3′ of CDR1 | Frequency |
|---|---|---|---|
| Vκ1 | 4 (FR1) | 3 (FR2) | 27% |
| Vκ2 | 7 (FR1) | 11 (FR2) | 4% |
| | 2 (June) | 7 (Junc) | |
| Vκ3 | 2 (June) | 13 (FR2) | 24% |
| Vκ4 | 2 (FR1) | 9 (FR2) | 6% |
| Vλ1 | 2 (FR1) | 11 (FR2) | 18% |
| Vλ2 | 3 (FR1) | 9 (FR2) | 13% |
| Vλ3 | 5 (FR1) | 9 (FR2) | 11% |

Table 3 Legend:
The column labeled “Frequency” indicates the frequency of each VL chain isotype based on the findings of de Wildt et al.(2000) Eur. J. Immunol. 30:254-261.
“June” indicates that the sites were identified within the junction region that includes a less-diverse region within CDR1 as well as the adjoining framework.

Table 4 provides further details as an example of the sites identified for one of the isotypes, VκII.

TABLE 4
Sites useful for cleaving nucleic acid in the region of VκII CDR1

| 3′ FR1 | 5′FR2 | 3′FR1 + 5′CDR1 | 3′CDR1 + 5′FR2 |
|---|---|---|---|
| Cfr10I | Acc65I | MaeI | Acc65I |
| Eco56I | Asp781I | PstI | Asp781I |
| HpaII | BanI | | BanI |
| MspI | BspMI | | KpnI |
| NaeI | BstNI | | NlaIV |
| HaeIII | EcoRII | | PstI |
| MnII | KpnI | | RsaI |
| | NlaIV | | |
| | PstI | | |
| | RsaI | | |
| | ScrFI | | |

Table 4 Legend:
Sites in the 3′ region of framework 1(FR1) are in the column labeled “3′ FR1.” Sites in the 5′ region of framework 2 (FR2) are in the column labeled “5′ FR2.” Sites in either of these two regions are external to CDR1. In addition, sites that closely straddle the framework-CDR1 boundaries are in the columns labeled “3′ FR1 + 5′CDR1” and “3′CDR1 + 5′FR2.”

After sites are identified for each particular family of the repertoire, restriction enzymes that cleave all members of the same family in a substantially similar location were identified. One enzyme was identified for the 5′ region of CDR1 and one for the 3′ region.

Two sets of restriction enzymes were identified for releasing diverse oligonucleotides that encode CDR1 of PHI. The first set, MnlI and KpnI, releases a fragment of 57 nucleotides. Based on the estimation provided by Equation 2, these diversity oligonucleotides have a Tₘ of between 66.6 to 68.8° C. The second set, NaeI and BstNI, releases diverse oligonucleotides of 81 nucleotides in length. Its Tₘ is in the range of 73.2 to 74.8° C. The results for particular germ line segments (DPK15, DPK12, DPK18, DPK19, and DPK28) or rearranged Vκ genes (as for PH1) are listed in Table 5.

Information about restriction enzyme sites can be found from a variety of sources including catalogs published by commercial providers of restriction enzymes, e.g., New England Biolabs (MA, USA). These providers also have on-line resources that can be accessed using the Internet. These sources provide information about buffer conditions, temperature, and other reaction parameters for numerous restriction enzymes.

TABLE 5
Examples for the VκII family of diverse oligonucleotides
derived by digestion with the indicated enzyme pair of the
indicated germ line segment/clone using cleavage-directing
oligonucleotides at the 5′ and 3′ end of the CDR1.

| Enzyme | Derived | | | |
|---|---|---|---|---|
| pair | from | Sequence | Length | Tₘ* |
| MnlI + | PH1 | 5′-TGCAGGTCTAGTCAGAGCCTCCTGCATAGT | 57 | 68.1° C. |
| KpnI | | AATGGATACACCTATTTGGATTGGTAC-3′ | | |
| | | (SEQ ID NO:7) | | |
| MnlI + | DPK15 | 5′-TGCAGGTCTAGTCAGAGCCTCCTGCATAGT | 57 | 67.4° C. |
| KpnI | | AATGGATACAACTATTTGGATTGGTAC-3′ | | |
| | | (SEQ ID NO:8) | | |
| MnlI + | DPK18 + | 5′-TGCAGGTCTAGTCAAAGCCTCGTATACAGT | 57 | 66.6° C. |
| KpnI | DPK19 | GATGGAAACACCTACTTGAATTGGTTT-3′ | | |
| | | (SEQ ID NO:9) | | |
| MnlI + | DPK28 + | 5′-TGCAAGTCTAGTCAGAGCCTCCTGCATAGT | 57 | 67.4° C. |
| KpnI | DPK12 | GATGGAAAGACCTATTTGTATTGGTAC-3′ | | |
| | | (SEQ ID NO:10) | | |
| MnlI + | DPK16 | 5′-TGCAGGTCTAGTCAAAGCCTCGTACACAGT | 57 | 68.8° C. |
| KpnI | | GATGGAAACACCTACTTGAGTTGGCTT-3′ | | |
| | | (SEQ ID NO:11) | | |
| NaeI + | PH1 | 5′-GGCCTCCATCTCCTGCAGGTCTAGTCAGAG | 81 | 73.8° C. |
| BstNI | | CCTCCTGCATAGTAATGGATACACCTATTTGGA | | |
| | | TTGGTACCTGCAGAAGCC-3′ | | |
| | | (SEQ ID NO:12) | | |
| NaeI + | DPK15 | 5′-GGCCTCCATCTCCTGCAGGTCTAGTCAGAG | 81 | 73.2° C. |
| BstNI | | CCTCCTGCATAGTAATGGATACAACTATTTGGA | | |
| | | TTGGTACCTGCAGAAGCC-3′ | | |
| | | (SEQ ID NO:13) | | |
| NaeI + | DPK18 + | 5′-GGCCTCCATCTCCTGCAGGTCTAGTCAAAG | 81 | 73.2° C. |
| BstNI | DPK19 | CCTCGTATACAGTGATGGAAACACCTACTTGAA | | |
| | | TTGGTTTCAGCAGAGGCC-3′ | | |
| | | (SEQ ID NO:14) | | |
| NaeI + | DPK28 + | 5-′GGCCTCCATCTCCTGCAAGTCTAGTCAGAG | 81 | 73.2° C. |
| BstNI | DPK12 | CCTCCTGCATAGTGATGGAAAGACCTATTTGTA | | |
| | | TTGGTACCTGCAGAAGCC-3′ | | |
| | | (SEQ ID NO:15) | | |
| NaeI + | DPK16 | 5′-GGCCTCCATCTCCTGCAGGTCTAGTCAAAG | 81 | 74.8° C. |
| BstNI | | CCTCGTACACAGTGATGGAAACACCTACTTGAG | | |
| | | TTGGCTTCAGCAGAGGCC-3′ | | |
| | | (SEQ ID NO:16) | | |

*Tₘ was calculated for a DNA-DNA hybrid without mismatch.

Sites were first identified for the nucleic acid sequence encoding the PH1 antibody. The analysis was then extended to other VκII family members. Two sets of restriction enzymes were identified for releasing diverse oligonucleotides. The first set MnlI and KpnI releases a fragment of 57 nucleotides. Based on the Baldino estimation, these diverse oligonucleotides have a Tₘ of between 66.6 to 68.8° C. The second set, NaeI and BstNI releases a larger fragment, about 81 nucleotides. Its Tₘ is correspondingly higher, in the range of 73.2 to 74.8

Depending on the desired Tₘ, either of the two enzyme pairs (MnlI+KpnI or NaeI+BstNI) can be used to generate diverse oligonucleotides that encode CDR1 from human Vκ light chains.

### Example 3

### Cleavage of Human Light Chain Immunoglobulin Genes to Construct Diverse Oligonucleotides

The method described in this Example is set forth in FIG. 1. mRNA is prepared from cells that express an immunoglobulin, e.g., IgG or IgM. RACE is used to amplify nucleic acids that encode the immunoglobulins using primers specific for the constant region within the immunoglobulin light chain genes (kappa). After the reverse transcription reaction, amplification is performed with a 5′-biotinylated oligonucleotide and the primer based in the constant region. The top strand of the resulting ds-RACE amplified cDNA is labeled with biotin. Primers specific to immunoglobulin heavy and light chains are used for the RACE protocol. This double-stranded cDNA is attached to a solid support, e.g., streptavidin magnetic beads (Item 1 of FIG. 1). The cDNA is then denatured with alkali such that the top strand remains attached to the beads (Item 2 of FIG. 1). The beads are washed to remove the lower nucleic acid strand.

To provide a mixture of diverse oligonucleotides with a high homology to germ line segments derived from the human V-kappa II family, the following procedure can be applied. The first cleavage-directing oligonucleotide (abbreviated “CDO”) is annealed (Item 3 of FIG. 1). The sequence of the first cleavage-directing oligonucleotide is 5′-CTG CCC TGG CTT CTG CAG GTA CCA-3′ (SEQ ID NO:17). The appropriate KpnI restriction enzyme is added to cleave the cDNA top strand in the duplex region formed by the first cleavage-directing oligonucleotide (Item 4 of FIG. 1). The reaction mixture is then washed from the beads to remove the 3′ most terminal fragment created by the cleavage. Then a second cleavage-directing oligonucleotide (5′-GCA GGA GAT GGA GGC CGG CTC TCC-3′, SEQ ID NO:18) is annealed (Item 5 of FIG. 1) and then the MnlI restriction enzyme appropriate for the second oligonucleotide is added (Item 6 of FIG. 1). The cDNA top strand is cleaved again, releasing the region between the site cleaved by the first enzyme and the site cleaved by the second enzyme. This region includes the sequence encoding the CDR1. Accordingly, the reaction mixture is removed from the beads and collected (Item 7 of FIG. 1). The released region is concentrated and stored as a pool of diverse oligonucleotides.

The same RACE-material can be used in a similar manner to build pools of diverse oligonucleotides for the CDR1, 2 or 3 of different human Vκ families.

### Example 4

### Preparation of Genetic Material Encoding Repertoires of Naturally Diversified V-Genes from Human Peripheral Lymphocytes, by RACE and Amplification

Naturally diversified antibody gene pools are readily accessible by isolating them, for example, from somatically mutated V-genes of the B-cells of human subjects. Depending on the source of the B-cells, the antibody genes can display a certain level of mutations in FR and CDR regions. Diverse pools of CDRs can be isolated from such repertoires, for example, as follow. Here, we describe the first step of an exemplary procedure (exemplified for the light chain only, but a similar protocol can be followed for the heavy chain): the RACE (Rapid Amplification of cDNA Ends) to isolate V-gene pools from human PBLs for further manipulations.

Two separate repertoires of human-kappa chain and human lambda-chain mRNAs were prepared by treating poly(A+) RNA isolated from five healthy volunteers with calf intestinal phosphatase to remove the 5′-phosphate from all molecules that contain them, such as ribosomal RNA, fragmented mRNA, tRNA and genomic DNA. Full length mRNA (containing a protective 7-methyl cap structure) is unaffected. The RNA is then treated with tobacco acid pyrophosphatase to remove the cap structure from full length mRNAs leaving a 5′-monophosphate group.

Full length mRNA's were modified with an adaptor at the 5′ end RNArace (5′-CGACUGGAGCACGAGGACACUGACAUGGACUGAAGGAGUAGAAA-3′; SEQ ID NO:19) and then reversed transcribed. The cDNA is used for amplification with a 5′ primer OUTINV (5′-CGACTGGAGCACGAGGACACTGA-3′; SEQ ID NO:20) (also called later the GeneRACE™ adapter) and a 3′ primer complementary to a portion of the construct (constant) region of kappa, cKHyAD2 (5′-ACACTCTCCCCTGTTGAAGCTCTTTGTGAC-3′; SEQ ID NO:21) and lambda, clamHyAD (5′-TGAACATTCTGTAGGGGCCACTGTCTTCTCC-3′; SEQ ID NO:22) using the GeneRACE™ method and kit (Invitrogen). A 5′ biotinylated primer complementary to the adaptor BioININV (5′-Bio-GGACACTGACATGGACTGAAGGAGTA-3′; SEQ ID NO:23) and a 3′ primer complementary to a portion of the construct encoding the constant region of kappa, HuCkfornest (5′-AGGCCCTGATGGGTGACTTCG-3′; SEQ ID NO:24) and lambda, HuCLFornest (5′-TGCGTGACCTGGCAGCTGTAGC-3′; SEQ ID NO:25) were used. The biotinylated product can be used for capturing to a streptavidin-coated surface. After denaturation of the strands, the top strand will remain bound to the streptavidin-coated surface.

As an alternative, a non-labeled primer complementary to the GeneRACE™ adapter and a biotinylated primer complementary to a portion of the construct region can be used. In this way, the lower strand of the amplified products can be used for capturing to a streptavidin-coated surface.

### Example 5

### Design of Oligonucleotides for Cleavage and Preparation of a Human Vkappa-1 CDR1 Gene Pool by Site-Specific Cleavage in FR1 and FR2 Regions

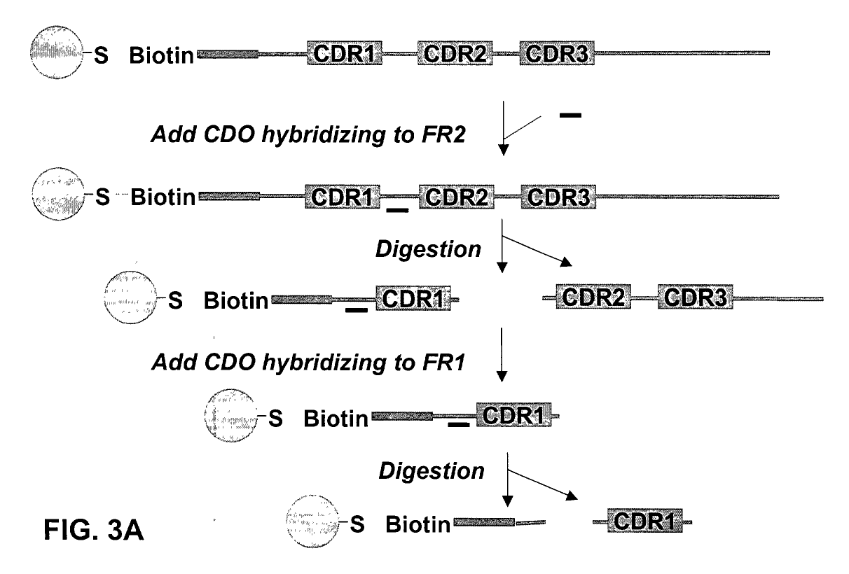

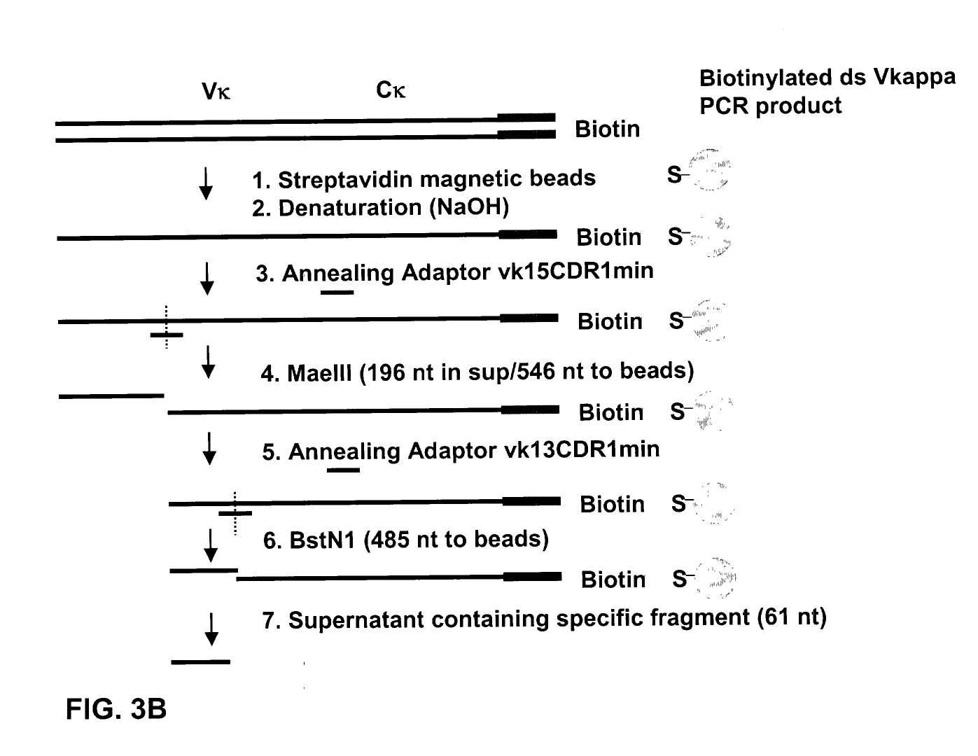

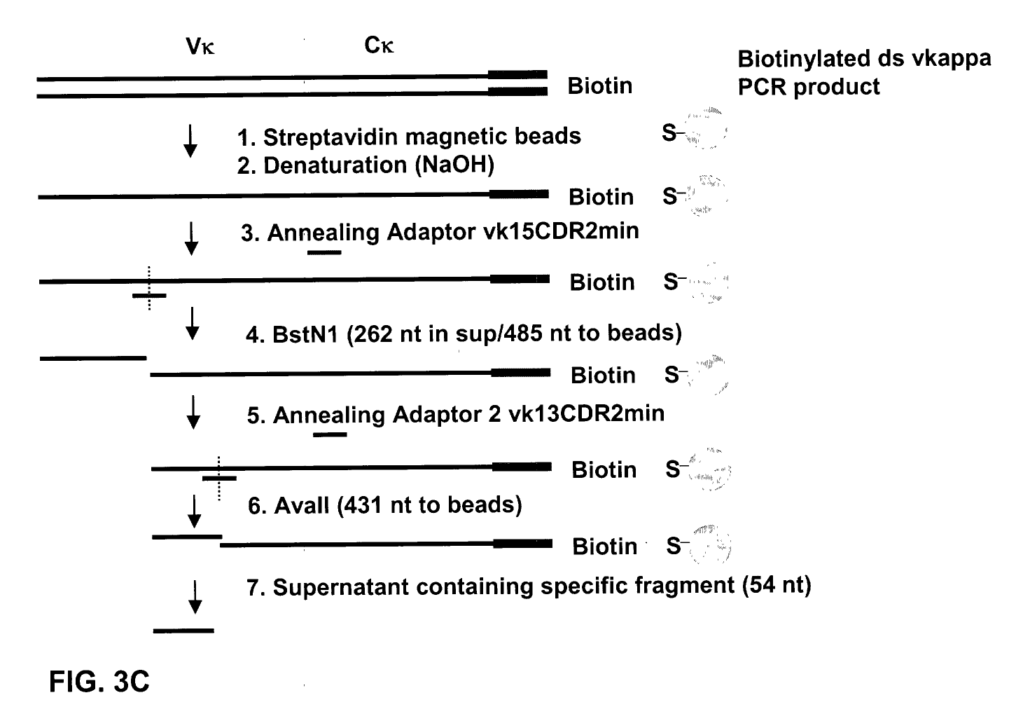

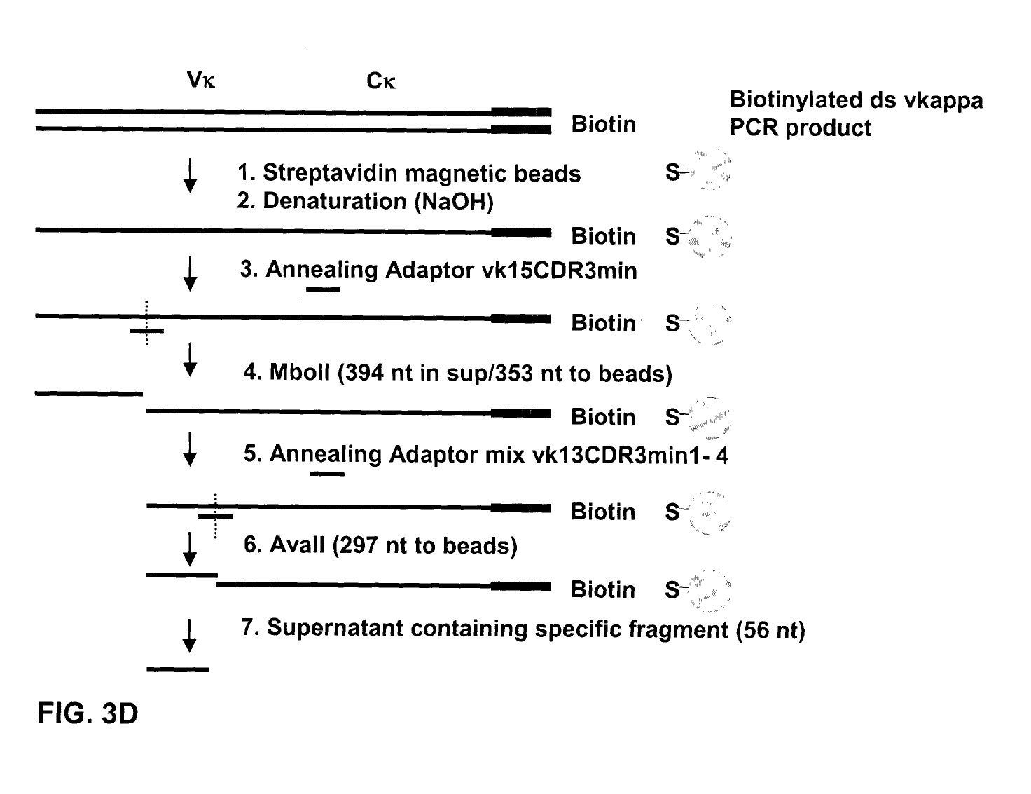

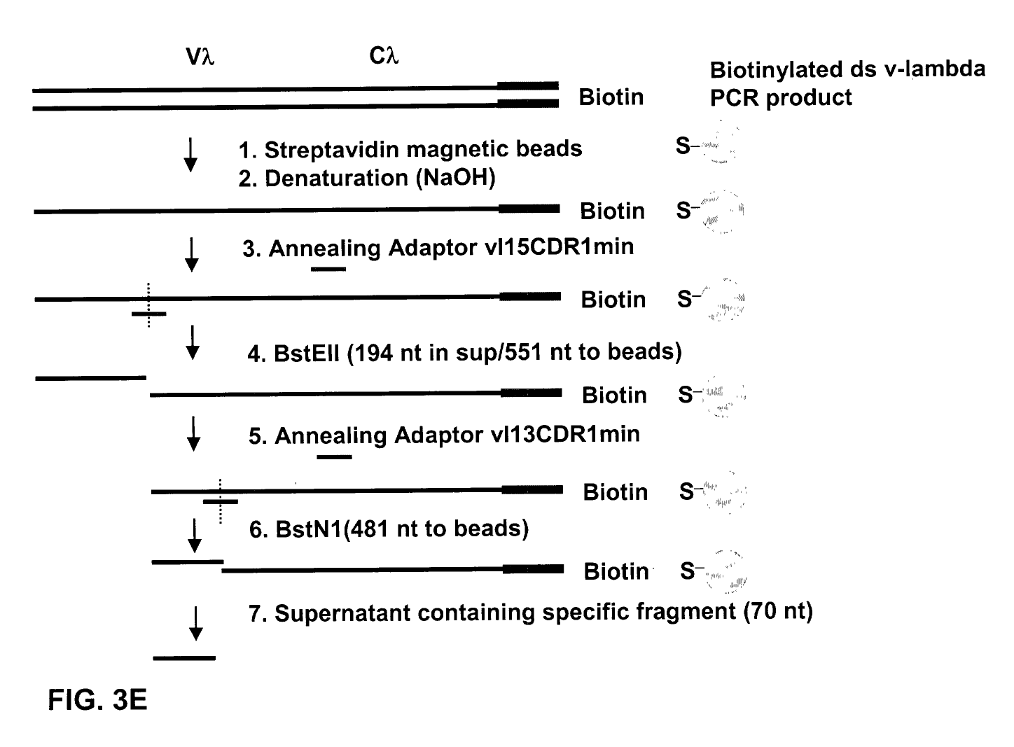

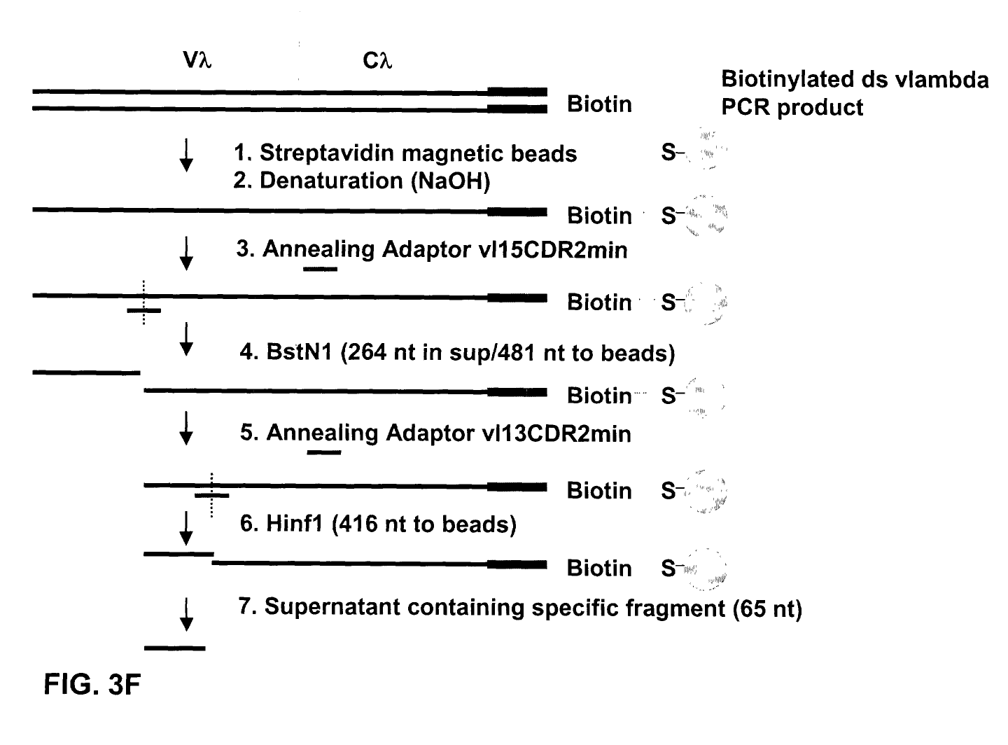

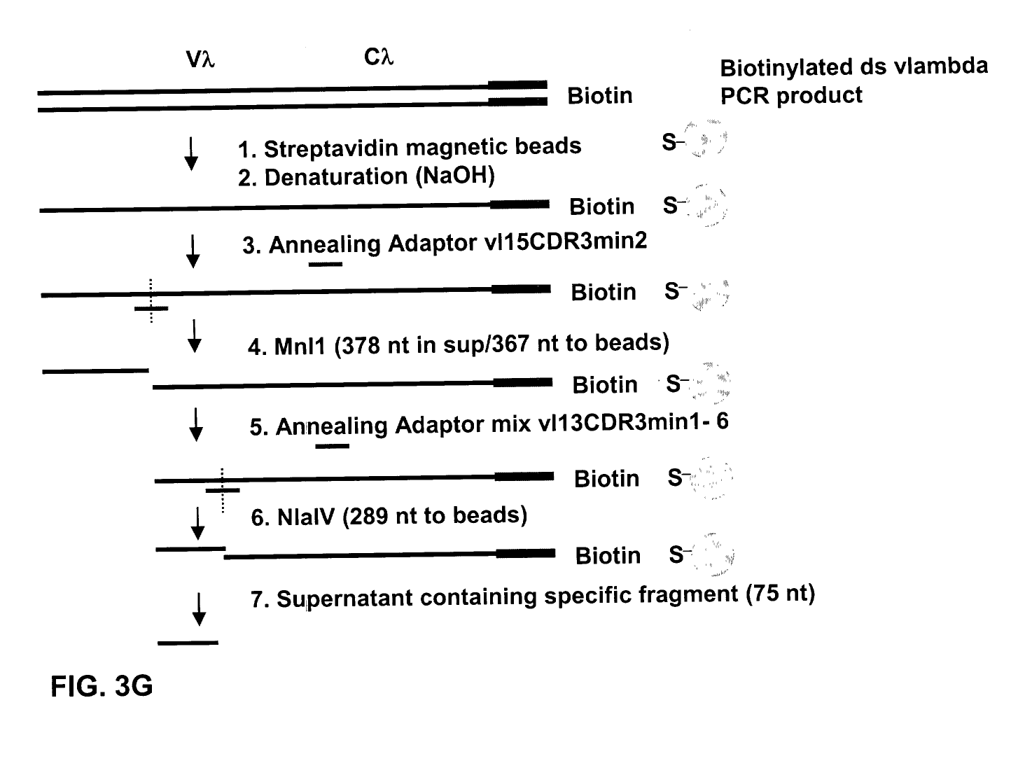

This example describes preparing oligonucleotides encoding CDR pools that are prepared by cleavage using a pair of cleavage-directing oligonucleotides. We designed oligonucleotides complementary to the regions bordering the CDR region of interest, and used these to direct the cleavage at specific sites surrounding the CDR of interest. The procedure to obtain the CDR1 oligonucleotide pool from the original V-gene pool, for example obtained by the procedure described in Example 4, is shown in FIG. 3A.

We analyzed bordering regions around CDR1 of human light chain family Vκ1 for the presence of naturally occurring restriction sites and we identified suitable enzyme pairs for cleavage-directing oligonucleotide mediated cutting (CDO-mediated cutting) (see, e.g., WO 01/79481) of single clones and human light chain family Vκ1. For cleavage-directing oligonucleotide mediated cutting of Vkappa-1 CDR1, we designed oligonucleotide adapters directed to 3′ end of FR1 (vk15CDR1min) and 5′ end of FR2 (vk13CDR1min), (see FIG. 3B).

Approximately 10 micrograms (μg) of human kappa-chain gene RACE material (with ±742 bp for the full kappa genes) with biotin attached to 5′-end of lower strand was immobilized on 1000 microliters (μl) of Seradyn magnetic beads. The upper strand was removed by washing the DNA with 1000 μl of 0.1 M NaOH for 3 minutes. The beads were washed with 1000 μl 0.01M NaOH, neutralized two times with 1000 μl of 1×B&W buffer (5 mM Tris (pH 7.5), 0.5 mM EDTA, 1 M NaCl) and washed one time with 1×NEB buffer 2 (50 mM NaCl, 10 mM Tris-HCl, 10 mM MgCl₂, 1 mM dithiothreitol pH 7.9, (New England BioLabs, Beverly, Mass.). A short oligonucleotide adapter directed to 3′ end of Vκ1-FR1, vk15CDR1 min (5′-TGGTATCAGCAGAAACCAGGGAAA-3′; SEQ ID NO:26) was added in 40 fold molar excess in 1000 μl of NEB buffer 2 to the beads. The mixture was incubated at 90° C. for 5 minutes then cooled down to 55° C. over 30 minutes. Excess oligonucleotide was washed away with 2 washes of 1×B&W buffer and one wash with 1×NEB2 buffer. Four units (0.8U/μg DNA) of MaeIII (Roche Diagnostics GmbH, Mannheim, Germany) were added in MaeIII buffer (20 mM Tris-HCl, 275 mM NaCl, 6 mM MgCl₂, 7 mM 2-Mercaptoethanol, pH 8.2) and incubated for 30 minutes at 60° C. A fragment of 196 nt was cleaved and released into the supernatant. The beads containing a fragment of 546 nt were washed with one wash of 1×B&W buffer and one wash with 1×NEB2 buffer. Subsequently, a second short oligonucleotide adapter directed to 5′ end of Vκ1-FR2, vk13CDR1min (5′-GGAGACAGAGTCACCATCACTTGC-3′; SEQ ID NO:27) was added in 40 fold molar excess in 800 μl of NEB buffer 2 (10 mM Tris-HCl, 10 mM MgCl₂, 50 mM NaCl, 1 mM dithiothreitol pH 7.9) to the beads. The mixture was incubated at 90° C. for 5 minutes then cooled down to 50° C. over 30 minutes. Excess oligonucleotide was washed away with two washes of 1×B&W buffer and one wash with 1×NEB2 buffer. The complex bound to the beads was cut with BstN1 (12.5U/μg DNA), (New England BioLabs, Beverly, Mass.) and incubated for 30 minutes at 60° C. The cleaved downstream DNA containing the 61-nt fragment was collected and separated on a 10% TBE-urea polyacrylamide gel (Bio-Rad, Hercules, Calif.). The fragment of 61 nucleotides was excised from the gel and eluted overnight at 37° C. in oligonucleotide-elution buffer (0.1% SDS, 0.5 M Ammonium Acetate, 10 mM). Subsequently, the supernatant was used for ethanol precipitation. The purified ssDNA fragments represent a pool of Vκ-CDR1 fragments with identical ends at 3′ and 5′ ends, and are an example of the ‘diverse oligonucleotides’ that can be used in a gene mutagenesis experiment. This pool was used for hybridization to a nucleic acid encoding an antibody clone of the Vκ1 family cloned in phage vector DY3F31 using different hybridization conditions (see Example 13). In similar experiments, oligonucleotides encoding Vkappa1-CDR2 and oligonucleotides encoding Vkappa1-CDR3 can be obtained using cleavage directing oligonucleotides pairs surrounding CDR2 and CDR3 of Vkappa1 (see FIGS. 3C and 3D).

### Example 6

### Design of Oligonucleotides for Cleavage and Preparation of a Human Vlambda-1 CDR1 Gene Pool by Site-Specific Cleavage in FR1 and FR2 Regions

We have analyzed bordering regions around CDR1 of human light chain family Vλ1 for the presence of naturally occurring restriction sites and we identified suitable enzyme pairs for use with oligonucleotide-directed cleavage (patent) of single clones and human light chain family Vλ1. For producing oligonucleotides encoding Vlambda-1 is CDR1, we designed cleavage-directing oligonucleotide specific for the nucleic acid encoding the 3′ end of FR1 (vl15CDR1min) and 5′ end of FR2 (vl13CDR1min), respectively (see FIG. 3E).

Approximately 10 μg of human lambda-chain gene RACE material with biotin attached to 5′-end of lower strand was immobilized on 1000 μl of Seradyn magnetic beads. The upper strand was removed by washing the DNA with 1000 μl of 0.1 M NaOH for 3 minutes. The beads were washed with 1000 μL 0.01M NaOH, neutralized two times with 1000 μl of 1×B&W buffer and washed 1 time with 1×NEB buffer 3 (100 mM NaCl, 50 mM Tris-HCl, 10 mM MgCl₂, 1 mM dithiothreitol pH 7.9, (New England BioLabs, Beverly, Mass.). A short oligonucleotide adapter vl15CDR1min (5′-GGGCAGAGGGTCACCATCTCCTGC-3′; SEQ ID NO:28) directed to 3′ end of Vλ1-FR1 was added in 40 fold molar excess in 1000 μl of NEB buffer 3 to the beads. The mixture was incubated at 90° C. for 5 minutes then cooled down to 55° C. over 30 minutes. Excess oligonucleotide was washed away with 2 washes of 1×B&W buffer and one wash with 1×NEB3 buffer. Fifty units (5U/μg DNA) of BstEII (New England BioLabs, Beverly, Mass.) were added in 1×NEB3 buffer and incubated for 1 hr at 60° C. After BstEII digestion, a fragment of 194 nt was cleaved off in the supernatant. The beads containing a fragment of 551 nt were washed with one wash of 1×B&W buffer and 1 wash with 1×NEB2 buffer. Subsequently, a second short oligonucleotide adapter directed to 5′ end of Vλ1-FR2, vk13CDR1min (5′-TGGTACCAGCAGCTTCCAGGAACA-3′; SEQ ID NO:29) was added in 40 fold molar excess in 1000 μl of NEB buffer 2 to the beads. The mixture was incubated at 90° C. for 5 minutes then cooled down to 55° C. over 30 minutes. Excess oligonucleotide was washed away with 2 washes of 1×B&W buffer and one wash with 1×NEB2 buffer. Six units (10U/μg DNA) of BstN1 were added in 1×NEB2 buffer and incubated for 1 hr at 60° C. The cleaved downstream DNA was collected and separated on a 10% TBE-urea polyacrylamide gel (Bio-Rad, Hercules, Calif.). The fragment of 70 nucleotides was excised from the gel, eluted overnight at 37° C. in oligonucleotide-elution buffer. Subsequently, the supernatant was used for ethanol precipitation.

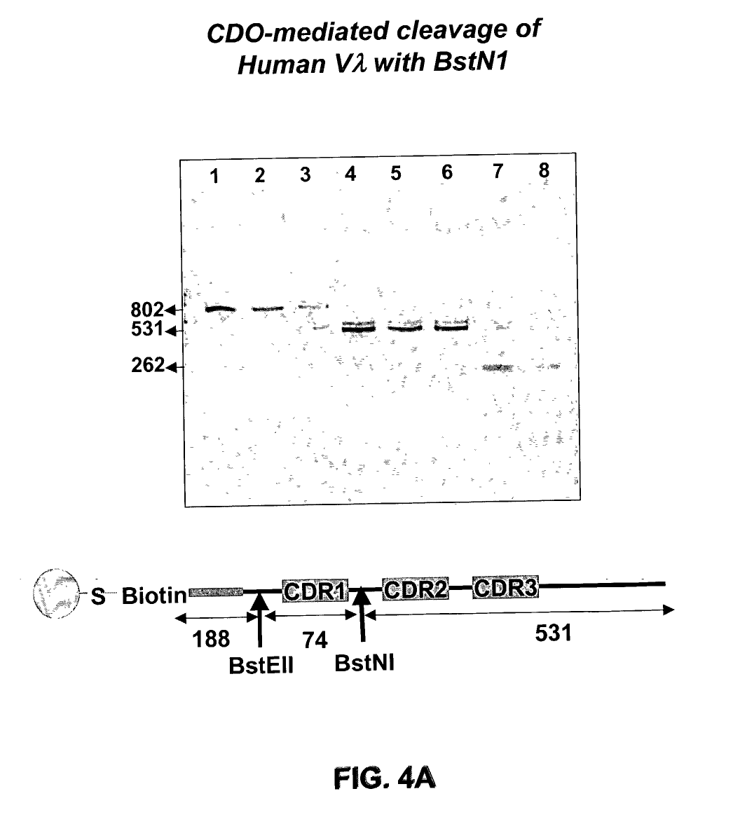

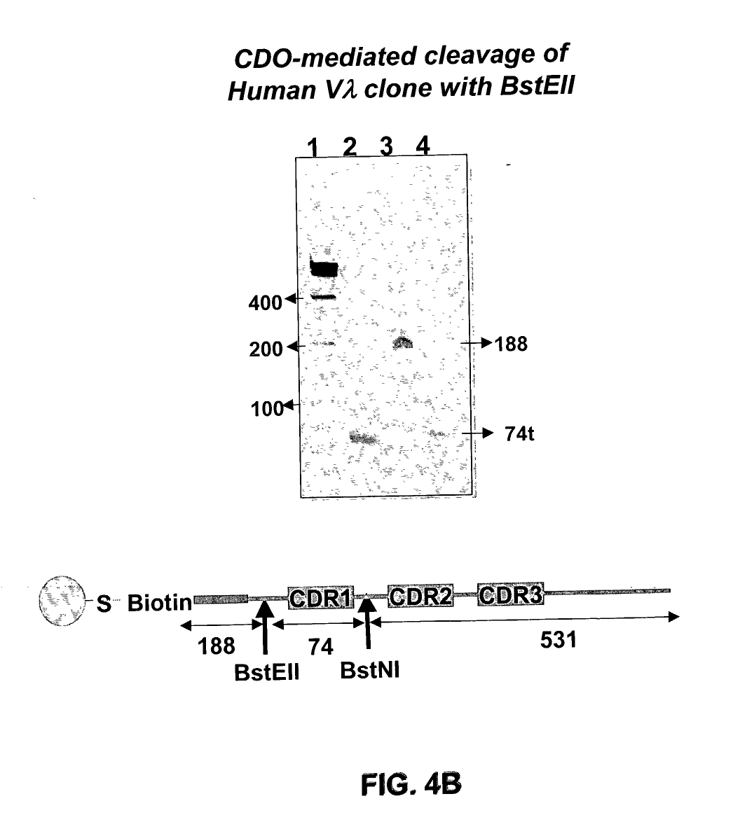

FIG. 4 shows an example of double CDO-mediated cleavage of a human Vλ clone with the use of cleavage directing oligonucleotides directed to top strand template (thus using the reverse setup, also using the reverse complement sequences of the cleavage-directing oligonucleotides and appropriate biotinylated RACE material (as in Example 4).

### Example 7

### Design of Oligonucleotides for Preparation of a Human Vlambda-1 CDR1 Gene Pool by PCR With Oligonucleotides in FR1 and FR2 Regions

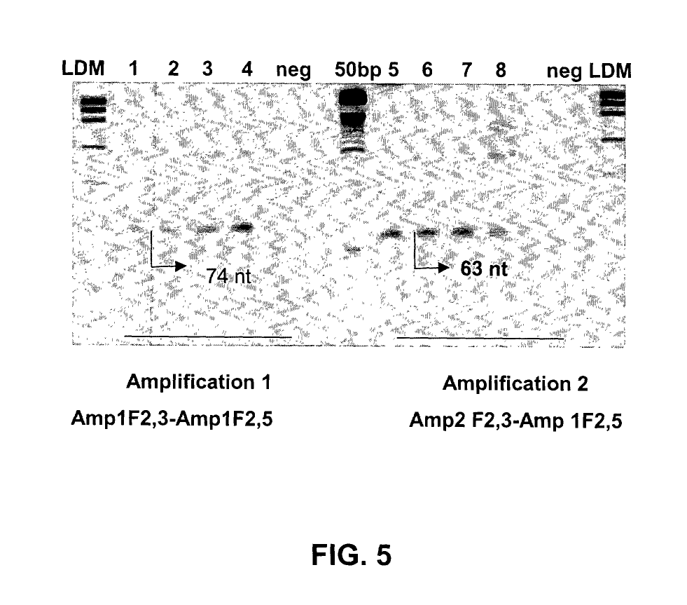

Alternatively to the use of CDO-mediated cleavage of ssDNA, amplification with oligonucleotides bordering the CDRs can be used to make CDR pools of any chosen type (heavy chain, lambda, kappa). We describe in this example the isolation of a human Vlambda-1 CDR1 gene pool made by PCR as an alternative to the use of CDO-cleaved fragments for introduction of hybridization-controlled mutagenesis of a template gene. The Vlambda repertoire obtained with the GeneRACE method (see Example 4) was used for amplification of a human Vlambda-1 CDR1 gene pool with forward primer AMP1F25CDR1 5′-GTCACCATCTCCTGC-3′ (SEQ ID NO:30), directed to 3′ end of Vlambda-1 FR1, and backward primer AMP2F23CDR1 (5′-GTACCAGTGTACATCATAAC-3′; SEQ ID NO:31), directed to 5′ FR2. The PCR mixture contained 50 ng template, 200 μM dNTPs, 0.2 μM of each forward and backward primer, 1 μl 50×Advantage 2 Polymerase Mix (Clontech, Palo Alto, Calif.) in 1×Advantage 2 PCR buffer (Clontech, Palo Alto, Calif.) in a total reaction volume of 50 μl. The PCR program consisted of one cycle of 3 minutes at 95° C. followed by 30 cycles of 95° C. for 30s, 58° C. for 45s, and 68° C. for 1 min. Following amplification, a fragment of 63 bp was obtained (see FIG. 5, right panel). As an alternative, a 74 bp fragment of a human Vlambda-1 CDR1 gene pool was obtained after amplification with forward primer AMP1F25CDR1 (5′-GTCACCATCTCCTGC-3′; SEQ ID NO:30), directed to 3′ end of Vlambda-1 FR1, and backward primer AMP1F23CDR1 (5′-GTACCAGTGTACATCATAAC-3′; SEQ ID NO:31), directed to the 3′ end of CDR1 and 5′ FR2 (see FIG. 5, left panel).

The purified ssDNA fragments represent a pool of Vλ-CDR1 fragments with identical ends at 3′ and 5′ ends, and are an example of the ‘diverse oligonucleotides’ that can be used in a gene mutagenesis experiment. This pool was used for hybridization with an antibody clone of the Vλ1 family cloned in phage vector using different hybridization conditions (see Example 13).

### Example 8

### Design of Oligonucleotides for Cleavage and Preparation of a Human Vlambda-1 CDR2 Gene Pool by Site-Specific Cleavage in FR2 and FR3 Regions

Similar to Example 6, we also analyzed regions bordering CDR2 of human light chain family Vλ1 for the presence of naturally occurring restriction sites and we identified suitable enzyme pairs for CDO cutting of single clones and human light chain family Vλ1. For CDO-mediated cutting of Vlambda-1 CDR2, we designed oligonucleotide adapters directed to 3′ end of FR2 (vl15CDR2 min) and 5′ end of FR3 (vl13CDR2 min), (see FIG. 3F).

Approximately 10 μg of human lambda gene RACE material with biotin attached to 5′-end of lower strand was immobilized on 1000 μl of Seradyn magnetic beads. The upper strand was removed by washing the DNA with 1000 μl of 0.1 M NaOH for 3 minutes. The beads were washed with 1000 μl 0.01M NaOH, neutralized 2 times with 1000 μl of 1×B&W buffer and washed 1 time with 1×NEB buffer 2. A short oligonucleotide adapter vl15CDR2 min (5′-CCAGGAACAGCCCCCAAACTCCTCATCTAT-3′; SEQ ID NO:32) directed to 3′ end of Vλ1-FR2 was added in 40 fold molar excess in 1000 μl of NEB buffer 3 to the beads. The mixture was incubated at 90° C. for 5 minutes then cooled down to 55° C. over 30 minutes. Excess oligonucleotide was washed away with 1 wash of 1×B&W buffer and one wash with 1×NEB2 buffer. Twenty-five units (5U/μg DNA) of BstN1 were added in 1×NEB2 buffer and incubated for 1 hr at 60° C. After BstN1 digestion, a fragment of 264 nt was cleaved off in the supernatant. The beads containing a fragment of 481 nt were washed with one wash of 1×B&W buffer and one wash with 1×NEB2 buffer. Subsequently, a second short oligonucleotide adapter directed to 5′ end of Vλ1-FR3, vl13CDR2 min (5′-GTCCCTGACCGATTCTCTGGC-3′; SEQ ID NO:33) was added in 40 fold molar excess in 1000 μl of NEB buffer 2 to the beads. The mixture was incubated at 90° C. for 5 minutes then cooled down to 50° C. over 30 minutes. Excess oligonucleotide was washed away with 2 washes of 1×B&W buffer and one wash with 1×NEB2 buffer. 12.5U units (33U/μg DNA) of Hinfl (New England BioLabs, Beverly, Mass.) were added in 1×NEB2 buffer and incubated for 1 hr at 50° C. The enzyme was heat-inactivated at 80° C. for 20 min. The cleaved downstream DNA was collected and separated on a 10% TBE-urea polyacrylamide gel (Bio-Rad, Hercules, Calif.). The fragment of 65 nucleotides was excised from the gel, eluted overnight at 37° C. in oligonucleotide-elution buffer. Subsequently, the supernatant was used for ethanol precipitation.

The purified ssDNA fragments represent a pool of Vλ-CDR2 fragments with identical ends at 3′ and 5′ ends, and are an example of the ‘diverse oligonucleotides’ that can be used in a gene mutagenesis experiment. This pool can be used for hybridization with one or more antibody templates.

### Example 9

### Design of Oligonucleotides for Cleavage and Preparation of a Human Vlambda-1 CDR3 Gene Pool by Site-Specific Cleavage in FR3 and FR4 Regions

We analyzed bordering regions around CDR3 of human light chain family Vλ1 for the presence of naturally occurring restriction sites and we identified suitable enzyme pairs for CDO cutting of single clones and human light chain family Vλ1. For CDO mediated-cutting of Vlambda-1 CDR3, we designed oligonucleotide adapters directed to 3′ end of FR3 (vl15CDR3 min) and 5′ end of FR4 (vl13CDR3 min1-6), (see FIG. 3G).

Approximately 10 μg of human Ig-lambda gene RACE material with biotin attached to 5′-end of lower strand was immobilized on 1000 ill of Seradyn magnetic beads. The upper strand was removed by washing the DNA with 1000 μl of 0.1 M NaOH for 3 minutes. The beads were washed with 1000 μl 0.01M NaOH, neutralized 2 times with 1000 μl of 1×B&W buffer and washed 1 time with 1×NEB buffer 2. A short oligonucleotide adapter vl15CDR3 min2 (5′-TCCAGGCTGAGGATGAGGCTGATTATTACTGC-3′; SEQ ID NO:34) directed to 3′ end of Vλ1-FR3 was added in 40 fold molar excess in 1000 μl of NEB buffer 2 to the beads. The mixture was incubated at 90° C. for 5 minutes then cooled down to 50° C. over 30 minutes. Excess oligonucleotide was washed away with 2 washes of 1×B&W buffer and one wash with 1×NEB3 buffer. Thirty-six units of BslI (New England BioLabs, Beverly, Mass.) were added in 1×NEB3 buffer and incubated for 1 hr at 55° C. The enzyme was heat-inactivated at 80° C. for 20 min. After BslI digestion, a fragment of 381 nt was cleaved off in the supernatant. The beads containing a fragment of 364 nt were washed with 1 wash of 1×B&W buffer and 1 wash with 1×NEB2 buffer. Subsequently, an equimolar mix of six oligonucleotide adapters directed to 5′ end of J-region (vl13CDR3 min1: 5′-TTCGGAACTGGGACCAAGGTCACC-3′; SEQ ID NO:35, vl13CDR3 min2: 5′-TTCGGCGGAGGGACCAAGCTGACC-3′; SEQ ID NO:36, vl13CDR3 min3: 5′-TTTGGTGGAGGAACCCAGCTGATC-3′; SEQ ID NO:37, vl13CDR3 min4: 5′-TTTGGTGAGGGGACCGAGCTGACC-3′; SEQ ID NO:38, vl13CDR3 min5: 5′-TTCGGCAGTGGCACCAAGGTGACC-3′; SEQ ID NO:39 and vl13CDR3 min6: 5′-TTCGGAGGAGGCACCCAGCTGACC-3′; SEQ ID NO:40) was added in 40 fold molar excess in NEB buffer 2 to the beads. The mixture was incubated at 90° C. for 5 minutes then cooled down to 55° C. over 30 minutes. Excess oligonucleotide was washed away with 3 washes of 1×NEB4 buffer (50 mM potassium acetate, 20 mM Tris-acetate, 10 mM magnesium acetate, 1 mM dithiothreitol, pH 7.9). Four units of NlaIV were added in 1×NEB4 buffer and incubated for 1 hr at 50° C. The cleaved downstream DNA was collected and separated on a 10% TBE-urea polyacrylamide gel (Bio-Rad, Hercules, Calif.). The fragment of 75 nucleotides was excised from the gel, eluted overnight at 37° C. in oligo-elution buffer. Subsequently, the supernatant was used for ethanol precipitation.

The purified ssDNA fragments represent a pool of Vλ-CDR3 fragments with identical ends at 3′ and 5′ ends, and are an example of the ‘diverse oligonucleotides’ that can be used in a gene mutagenesis experiment. This pool can be used for hybridization with one or more antibody templates.

### Example 10

### Design of Synthetic Oligonucleotides for a Hybridization-Controlled Introduction of Mutations in an Antibody Template

In some instances it may be desirable to use oligonucleotides with synthetic diversity instead of using the naturally mutated antibody genes as a source of ‘diverse oligonucleotides’. The present example outlines the procedure contemplated by the applicants to be useful for the successful preparation of a mutant antibody library with such synthetic oligonucleotides. In the example, antibody genes cloned into a phagemid or phage vector are used for mutagenesis; this provides readily access to template material for a Kunkel-based mutagenesis (Example 12), but also other sources of V-genes/templates could be used.

In this example the design is given of oligonucleotides that can be used to diversify the heavy chain CDR1 and CDR2 regions, and in particular of antibodies that have been isolated from a semi-synthetic human Fab library. For the construction of these libraries, a synthetic CDR1 and 2 diversity was built in the 3-23 VH framework in a two step process: first, a vector containing a synthetic 3-23 VH framework was constructed, and then, a synthetic CDR1 and 2 was assembled and cloned into this vector. All antibodies selected from this library will therefore contain highly homologous FR regions in the VH. The oligonucleotides designed for a hybridization-controlled sequence variation are specifically applicable to antibody genes from this library; similar principles can be used to design oligonucleotides for antibodies from naive, immune or other synthetic libraries.

For hybridization controlled introduction of mutations in VH-CDR1, we designed a synthetic oligonucleotide VHCDR1HyAD (5′-AGCTTGGCGAACCCAMNNMNNMNNMNNMNNMNNMNNTCCGGAAGCAGCGCA-3′; SEQ ID NO:41) with the last four amino acid residues of FR1 and the CDR1 fully randomized for hybridization to top strand template. In this case also part of the FR1 was variegated, since this is often part of the structural ‘H1’ loop, frequently interacting with antigen or other CDR loops (thus likely to affect affinity).

For hybridization controlled introduction of mutations in VH-CDR2, we designed a synthetic oligonucleotide VHCDR2HyAD (5′-AACGGAGTCAGCATAMNNMNNMNNMNNMNNMNNMNNMNNMNNMNNAGAAACCCACTCCAA-3′; SEQ ID NO:42) with the first 10/17 amino acid residues of V3-23-CDR2 fully randomized for hybridization to top strand template. In this case the oligonucleotides will only variegate those residues that are also somatically the most frequently mutated.

The creation of the mutant antibody library will involve the following steps: (1) preparation of the circular single-stranded template DNA, derived from either one individual clone, a group of clones, of a selected population (in this case the sequence bordering the CDR1-2 regions will likely be identical or highly similar between clones, thus allowing hybridization to the same designed oligonucleotides); (2) hybridization of the template with one or both of the oligonucleotides indicated (termed Diverse Oligonucleotides, “DOs” in these specific examples); (3) separation of the non-hybridized oligonucleotides from the hybrid; (4) optionally (and if the design allows this), ligation of the two DOs (see also Example 11); (5) extension of the DNA strand from the DO-end(s) by a DNA polymerase and closing the circle with a ligase; and (6) introduction of the DNA into a host cell for recovery and/or expression of the variant molecules. The mutant antibody library can subsequently be screened to identify variants with improved affinity or altered expression level or stability (e.g., using standard methods such as filter-screening, ELISA screening of individually expressed variants), possibly after in vitro selection of variants from larger libraries (phage, yeast or ribosome display libraries).

### Example 11

### Examples of an Overall Mutagenesis Strategy for Antibodies

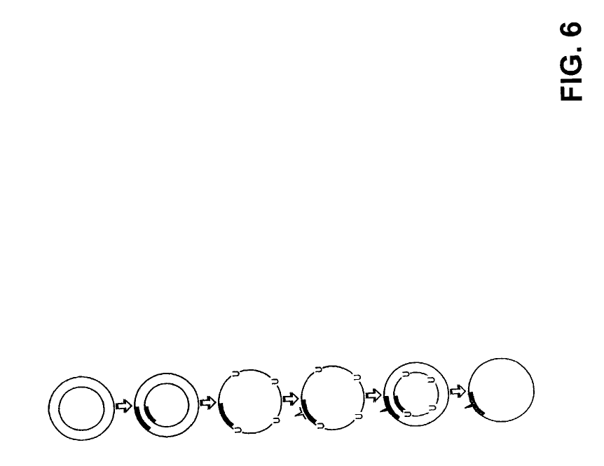

Antibody genes can be diversified using the controlled-hybridization mutagenesis strategy applied to individual CDR regions, followed by screening for affinity variants of the library of mutants. First a CDR pool is isolated from a source of V-genes with mutations present in at least a fraction of the genes, such as the V-genes from human peripheral blood lymphocytes. Methods for this are described in the previous examples. Secondly the CDR1 pool is hybridized to the template ssDNA, for example derived from the phagemid or phage vector that the antibody gene was cloned into. In a pilot experiment the average level of mutagenesis introduced versus hybridization conditions can be determined (by obtaining clones after the mutagenesis and sequencing), and a certain level can then be chosen for a larger-scale experiment, where more template and DO are used. From this hybridization mix, a library is made (e.g. via the Kunkel method), (see FIG. 6), which will now have a certain fraction of clones with mutations in the chosen CDR region. Typically a few conditions of stringency can be chosen, for example the calculated Tₘ and a few degrees below the Tₘ, and the mutants strands rescued. In some cases the CDR regions may be exchanged by variants based on other germ lines belonging to the same germ line as the template, leading to possibly undesired mutations or a consistent change of CDR length at lower hybridization temperature.

Alternatively antibody genes are diversified at two CDR regions at the same time, for example by hybridizing onto a given template a DO encoding a putatively mutated CDR1 region as well as a DO encoding another CDR regions. The positioning can be such that the two DO's will be spatially separated, with a larger region of ssDNA separating them (e.g. when targeting the CDR1 of the VL and CDR2 of the VH). Alternatively the DOs can be designed to hybridize to neighboring CDRs as non-overlapping fragments, and in some cases such that the 3′ end of one DO will be adjacent to the 5′ end of the other DO. In that case the addition of a DNA ligase can link the two hybridized regions covalently together. Such reaction may strengthen the interaction between template and DOs, and allow recovery of the mutant strand via traditional methods (Kunkel mutagenesis, FIG. 6) or methods normally not readily applicable to single DO-based mutagenesis (e.g. PCR with oligonucleotides, one each based in a different DO). When spatially separated, the same can be achieved by providing the complementary ssDNA that will hybridize between the two DOs. This can be extended to incorporate more DOs covering more than two CDR regions also.

In the case where two or more DOs are used for the mutagenesis, it will be important to take into account the difference in Tₘ for both interactions. In some cases the design of the DOs can be such that overall they have a Tₘ for most templates in the same range of one another. If this cannot be done, the ligation of neighboring DOs, or, more general, the ligation of one DO to a given second ssDNA fragment (either a DO or a unique DNA sequence that is complementary with the template), can be used to alter the Tₘ (of the ligated complex) in subsequent experiments. This allows more stringent conditions to be applied for further mutagenesis.

In the previous examples, the area of mutagenesis focused on the CDR regions. It is also possible to use controlled hybridization to generate mutations in the framework regions. The same site-directed mutagenesis strategy as for the CDRs can be followed when the objective is to target residues within the FR regions, for example for improvement of affinity, stability or expression level.

For the mutagenesis of the heavy and light chain CDR3 regions, the diversity in the somatic human antibody repertoire present is tremendous with regards to both length and sequence. This is particularly the case for the heavy chain CDR3 region, with over 10²³ different sequences. For many antibody templates, there will be a limited set of point mutation variants present in such somatic repertoire. For very long sequences, the probability of finding variants with a limited set of point mutations of the template sequence may be reduced. In such instances, one can use synthetic oligonucleotides with designed diversity, as a source of diversity for use in the hybridization reaction. The diversity can be encoded throughout the CDR3 regions, or be localized to certain residues if desired (see also Example 10).

### Example 12

### Preparation of V-Gene Template Material for Kunkel Mutagenesis

We describe here the preparation of template DNA for the Kunkel mutagenesis procedure (Kunkel et al, 1985). In this example we used a clone originating from a large non-immune human Fab phagemid library described in de Haard et al (JBC, 1999). This clone, Strep-F2, was obtained after selections on streptavidin. This clone was used for recloning from its original phagemid vector context into the DY3F63 vector (see, e.g., WO 00/70023) via ApaL1-Not1 restriction sites. Essentially this is a phage vector in which the antibody genes, in Fab format, are linked to the filamentous phage derived pill, and is thus used for making antibody display libraries. DNA from the DY3F63 phage vector was pretreated with ATP dependent DNase to remove chromosomal DNA and then digested with ApaL1-NotI. An extra digestion with Asc1 was performed to prevent self-ligation of the vector. The ApaL1/Not1 Strep-F2 Fab fragment was subsequently ligated to the vector DNA and transformed into competent E. Coli TG1 cells. Phage was prepared from this clone according to Marks et al, 1991. Uracil-incorporated phage was prepared according to the Muta-Gene® M13 in vitro mutagenesis kit (Bio-Rad, Hercules, Calif.). Single-stranded M13 phage DNA is isolated using the QIAprep™ Spin M13 Kit according to the manufacturer's instruction (Qiagen, Valencia, Calif.). Uracil-incorporated ssDNA was further used as template in the hybridization step (next example).

### Example 13

### Introduction of Mutations in a Vkappa-1 Template Using Hybridization With a Pool of CDR1 Segments: Hybridization, Cloning Via the Kunkel Method and Sequencing

We describe hybridization and strand extension reactions of CDO-cleaved products of the Vκ repertoire, hybridized to uracil-incorporated ssDNA from clone strep-A11 cloned in phage vector DY3F63 using the Kunkel method. Clone strep-A11 is an antibody binding to streptavidin and selected from a human Fab library; recloned similar as described for strep-F2 in Example 12, and finally its light chain belongs to the Vκ1 family. Five ng purified CDO-cleaved product (recovered from a urea-polyacrylamide gel and ethanol-precipitated) after cleaving a Vκ repertoire (Example 5) was added to 250 ng uracil incorporated ssDNA from clone Strep-A11 cloned in phage vector DY3F63 in a total reaction volume of 50 μl of 1× annealing buffer (20 mM Tris, pH 7.4, 2 mM MgCl₂, 50 mM NaCl). The mixture was incubated at 90° C. for 7 minutes and cooled down to 74° C. over 30 minutes. Different hybridization temperatures ranging from 70.5° C.-74° C. were used. After incubation, unbound fragments were separated from the hybrid-mix using a Microcon YM 100 according to the manufacturer's instructions (Millipore Corporation, Bedford, Mass.). The hybrid template was used for the mutagenesis reaction. The mutagenesis mixture contained 5 μl 5×T7 Polymerase reaction buffer (200 mM Tris, pH 7.5, 100 mM MgCl₂, 250 mM NaCl), 40 μM dNTPs, 1 μl (3U/μl) T4 DNA ligase (Promega Corporation, Madison), 2.5 μl 10×T4 DNA ligase buffer (300 mM Tris-HCl, pH 7.8, 100 mM MgCl₂, 100 mM DTT, 10 mM ATP), 1 μl (0.5U) T7 DNA polymerase diluted in 1×T7 DNA dilution buffer (20 mM potassium phosphate buffer, pH 7.4, 1 mM DTT, 0.1 mM EDTA, 50% glycerol), (USB Corporation, Cleveland, Ohio) in total reaction volume of 25 μl. The reaction was stabilized on ice for 5 min, incubated at 25° C. for 5 min, followed by 30 min incubation at 37° C. Following the mutagenesis reaction, products were stored on ice. The mutagenized product mix was further incubated with 1 μl (0.2U/μl) uracil DNA glycosylase for 30 min at 37° C. to remove to uracil-incorporated parental strand. The reaction mixture was further purified with a Microcon Y100 (Millipore Corporation, Bedford, Mass.) according to the manufacturer's instructions. Five μl of the reaction mix was used for transformation into 50 μl competent E. Coli TG1 cells. After electroporation, 500 μl SOC medium (SOB with 2% glucose) was added. Transformation mixtures were plated onto 2×TY plates containing ampicillin and glucose (16 g/l bacto-tryptone, 10 g/l yeast extract, 5 g/l NaCl, 15 g/l bacto-agar, 100 μg/ml ampicillin, and 2% (w/v) glucose) and incubated overnight at 37° C. Individual clones were selected, insert PCR was performed with forward primer PlacPCRfw (5′-GTGAGTTAGCTCACTCATTAG-3′; SEQ ID NO:43)) and backward primer synGIII stumprev (5′-GCCATTTTCTCGTAGTCGAAG-3′; SEQ ID NO:44)). Subsequently, PCR products were used for sequence analysis with primer puc (5′-AGCGGATAACAATTTCACACAGG-3′; SEQ ID NO:45)) using an ABI automated DNA sequencer (PerkinElmer, Norwalk, Conn.).

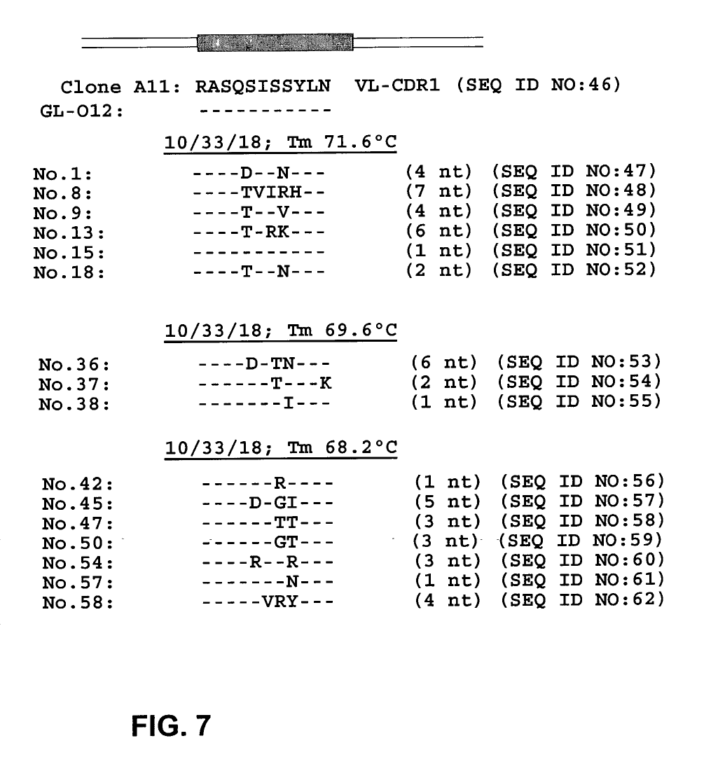

We used different hybridization temperatures, ranging from 68.2° C. to 71.6° C. We were able to successfully introduce different types of mutations at 6 amino acid positions of Vκ-CDR1 (FIG. 7) by the use of CDO-cleaved fragments of the Vκ repertoire for hybridization.

When further reducing the hybridization temperature, and using 5 ng purified CDO-cleaved product for hybridization with 250 ng uracil incorporated ssDNA from clone strep-A11 (primer:template ratio of 3:1), we successfully introduced different types of mutations at all 11 amino acid positions of Vκ-CDR1 in a range of 3° C.-8° C. from Tₘ. Thus, the lower hybridization temperature allowed more variation to be introduced. Apart from mutations introduced at the CDR1 region, nucleotide changes were also introduced in both FR1 and FR2 regions (due to overlap at FR regions). These nucleotide changes corresponded to the germline diversity or somatic mutations in the FR regions. The number of clones with mutations introduced varied depending on the hybridization Tₘ used.

### Example 14

### Introduction of Mutations in a Vlambda-1 Template Using Hybridization With a Pool of CDR1 Segments: Hybridization, Cloning Via the Kunkel Method and Sequencing

We describe hybridization and strand extension of double CDO-cleaved fragments of the Vλ repertoire hybridized to uracil-incorporated ssDNA from clone strep-F2 cloned in DY3F63 phage vector using the Kunkel method. Five ng purified CDO-cleaved product (recovered from a urea-polyacrylamide gel and ethanol-precipitated) after cleaving a Vλ repertoire was added to 250 ng uracil incorporated ssDNA from clone Strep-F2 in DY3F63 in a total reaction volume of 50 μl of 1× annealing buffer (20 mM Tris, pH 7.4, 2 mM MgCl₂, 50 mM NaCl). The mixture was incubated at 90° C. for 7 minutes then cooled down to the 74° C. over 30 minutes. Different hybridization temperatures ranging from 70.5° C.-74° C.) were used. After incubation, unbound fragments were separated from the hybrid-mix using a Microcon YM 100 according to the manufacturer's instructions (Millipore Corporation, Bedford, Mass.). The hybrid template was used for the mutagenesis reaction. The mutagenesis mixture contained 5 μl 5×T7 Polymerase reaction buffer (200 mM Tris, pH 7.5, 100 mM MgCl₂, 250 mM NaCl), 40 μM dNTPs, 1 μl (3U/μl) T4 DNA ligase (Promega Corporation, Madison), 2.5 μl, 10×T4 DNA ligase buffer (300 mM Tris-HCl, pH 7.8, 100 mM MgCl₂, 100 mM DTT, 10 mM ATP), 1 μl (0.5U) T7 DNA polymerase diluted in 1×T7 DNA dilution buffer (20 mM potassium phosphate buffer, pH 7.4, 1 mM DTT, 0.1 mM EDTA, 50% glycerol), (USB Corp., Cleveland, Ohio) in total reaction volume of 25 μl. The reaction was stabilized on ice for 5 min, incubated at 25° C. for 5 min, followed by 30 min incubation at 37° C. Following the mutagenesis reaction, products were stored on ice. The mutagenized product mix was further incubated with 1 μl (0.2U/μl) uracil DNA glycosylase for 30 min at 37° C. to remove to uracil-incorporated parental strand. The reaction mixture was further purified with a Microcon Y100 (Millipore Corporation, Bedford, Mass.) according to the manufacturer's instructions. 5 μl of the reaction mix was used for transformation into 50 μl competent E. coli TG1 cells. After electroporation, 500 μl SOC medium (20 g/l bacto-tryptone, 5 g/l yeast extract, 5 g/l NaCl, and 20 mM glucose) was added. Transformation mixtures were plated onto 2×TY plates containing ampicillin and glucose (16 g/l bacto-tryptone, 10 g/l yeast extract, 5 g/l NaCl, 15 g/l bacto-agar, 100 μg/ml ampicillin, and 2% (w/v) glucose) and incubated overnight at 37° C. Individual clones were selected, insert PCR was performed with forward primer PlacPCRfw (5′-GTGAGTTAGCTCACTCATTAG-3′; SEQ ID NO:43) and backward primer synGIII stumprev (5′-GCCATTTTCTCGTAGTCGAAG-3′; SEQ ID NO:44). Subsequently, PCR products were used for sequence analysis with forward primer puc (5′-AGCGGATAACAATTTCACACAGG-3′; SEQ ID NO:45) using an ABI automated DNA sequencer (PerkinElmer, Norwalk, Conn.).

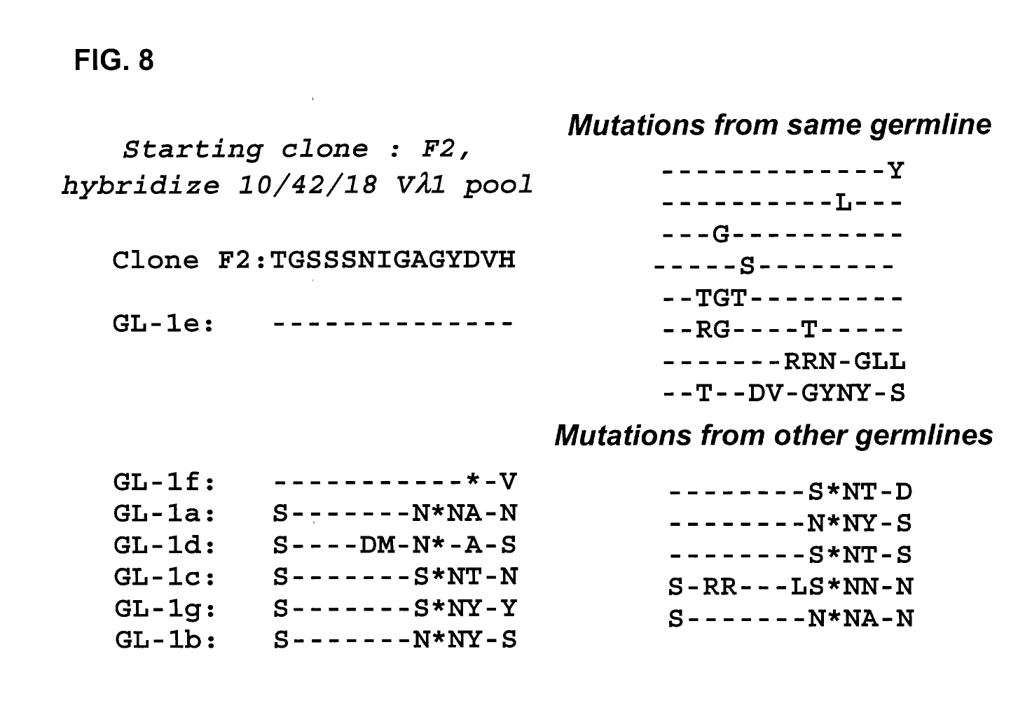

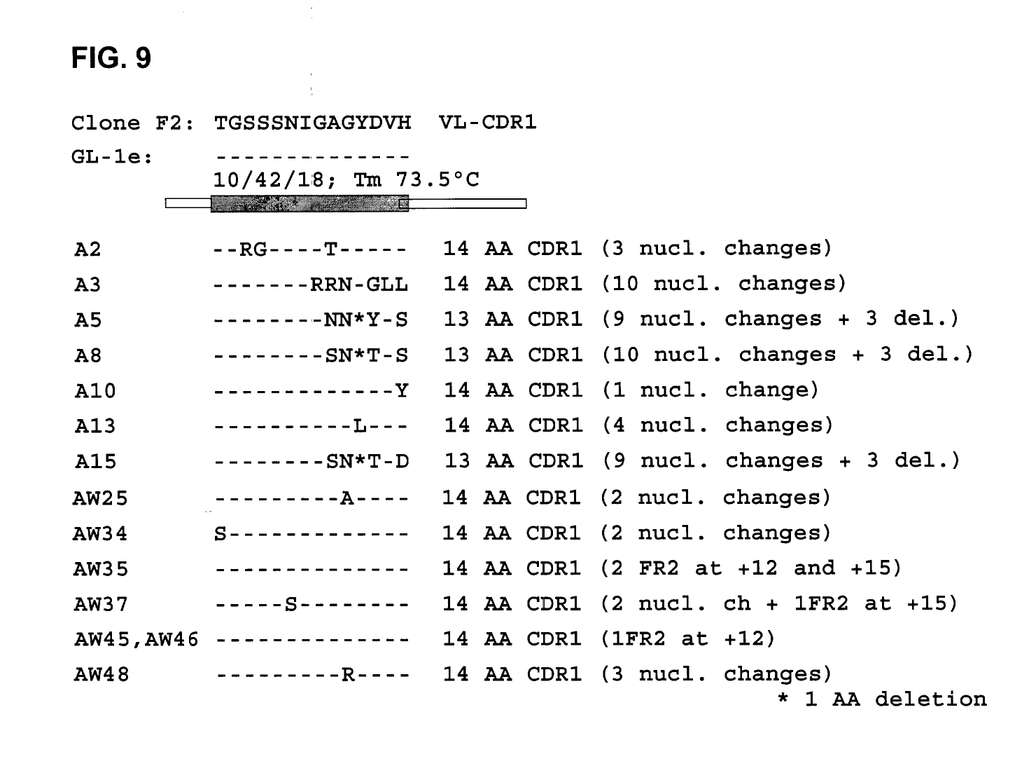

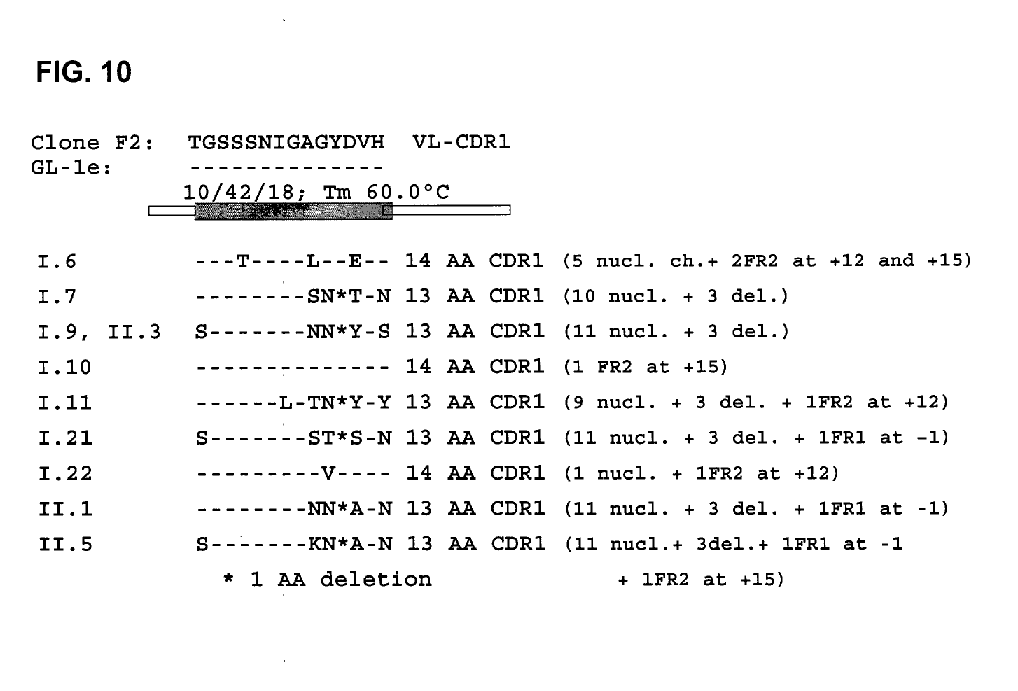

We used different hybridization temperatures, ranging from 70° C. to 74° C. No nucleotide changes were found at the highest Tₘ which favored a perfect match with the template gene Strep-F2 (−74° C.). Using hybridization temperatures close to the calculated Tₘ (73.0° C.-73.5° C.), both nucleotide changes and deletions were introduced. (see FIG. 9). As expected, the frequency of clones with nucleotide changes introduced (=14 AA CDR1) was higher than the number of clones with deletions introduced (=13 AA CDR1) using these high stringency hybridization conditions. We also tested lower hybridization temperatures based on the calculated Tₘ of other Vλ1 CDR1 germline sequences, (for the sequences see FIG. 8 and legend), (Tₘ from 70.5° C. to 71.1° C.=germline sequences with shorter CDR1 length of 13 amino acids). At these hybridization temperatures tested, we successfully introduced 3 nucleotide deletions (shorter CDR1) length according to the lower hybridization Tₘ, (see FIG. 10). The location of the deletions introduced in the CDR1 corresponds to the shorter germline CDR1 lengths. Apart from mutations introduced at the CDR1 region, nucleotide changes were also introduced in both FR1 and FR2 regions (due to overlap at FR regions). As expected, these nucleotide changes correspond to the germline diversity or somatic mutations in the FR regions. The number of clones with mutations and deletions introduced varied depending on the hybridization conditions tested.

### Example 15

### Creating Libraries of Mutants of a Single Template Created by Hybridization

Double CDO-cleaved CDR1 fragments of a human Vλ repertoire are hybridized to uracil-incorporated ssDNA from clone strep-F2 (from Vλ1 family) cloned in the phage vector DY3F63 using hybridization conditions that allow introduction of bias towards somatic mutations in the same germline (e.g., hybridization conditions close to the Tₘ of the Strep-F2 clone). Hybridization conditions were the same as those in Example 14 and include a hybridization temperature of 73.5° C. Under these conditions the mutations introduced in the CDR1 are mainly point mutations of the CDR1 region with the same germ line, and not mutations that would replace the germ line segment (with the concomitant deletion of one residue, e.g., as explained in Example 14, FIG. 8). We obtained a library of 3×10⁵ clones. Nucleic acid sequencing revealed that 1-5 nucleotide changes were introduced in the CDR1 of Strep-F2 in about 25% of the clones. This library is screened to identify variants with altered characteristics of the antibody investigated.

A number of embodiments of the invention have been described. Nevertheless, it will be understood that various modifications may be made without departing from the spirit and scope of the invention. Accordingly, other embodiments are within the scope of the following claims.

What is claimed:

1. A method of altering a nucleic acid strand, the method comprising:
a) providing i) a template nucleic acid strand and ii) a plurality of diverse nucleic acids;
b) annealing a cleavage-directing oligonucleotide to each of the diverse nucleic acids of the plurality to form a cleavable region on each of the diverse nucleic acids;
c) cleaving the cleavable region of each diverse nucleic acid to form a plurality of diversity oligonucleotides;
d) combining the plurality of diversity oligonucleotides and the template nucleic acid strand in a mixture;
e) subjecting the mixture to conditions such that only a subset of the plurality of diversity oligonucleotides anneal to the template nucleic acid strand; and
f) extending and/or ligating an annealed oligonucleotide of the subset to form an altered nucleic acid strand that incorporates the annealed oligonucleotide and a sequence complementary to the template nucleic acid strand.

2. A method of altering a plurality of template nucleic acid strands, the method comprising:
a) providing i) a plurality of template nucleic acid strands and ii) a plurality of diverse nucleic acids;
b) annealing a cleavage-directing oligonucleotide to each of the diverse nucleic acids of the plurality of diverse nucleic acids to form a cleavable region on each of the diverse nucleic acids;
c) cleaving the cleavable region of each diverse nucleic acid to form a plurality of diversity oligonucleotides;
d) combining the plurality of diversity oligonucleotides and the plurality of template nucleic acid strands in a mixture;
e) subjecting the mixture to conditions such that only a subset of the plurality of diversity oligonucleotides anneal to one or more template nucleic acid strands of the plurality; and
f) extending and/or ligating one or more annealed oligonucleotides of the subset to form one or more altered nucleic acid strands, each incorporating at least one of the annealed oligonucleotides and a sequence complementary to a template nucleic acid strand from the plurality.

3. A method of altering an immunoglobulin variable domain coding sequence, the method comprising:
a) providing i) a template nucleic acid strand that comprises a nucleic acid sequence that encodes an amino acid sequence comprising an immunoglobulin variable domain, or a complement thereof, and ii) a plurality of diversity oligonucleotides, each diversity oligonucleotides comprising a nucleic acid sequence that encodes an amino acid sequence that includes a single complementarity determining region (CDR), and a portion of each framework region flanking the CDR, or a complement thereof;
b) combining the plurality of diversity oligonucleotides and the template nucleic acid strand in a mixture;
c) subjecting the mixture to conditions such that only a subset of the plurality of diversity oligonucleotides anneal to the template nucleic acid strand; and
d) extending and/or ligating an annealed oligonucleotide of the subset to form an altered nucleic acid strand that is partially complementary to the template nucleic acid strand, and incorporates the annealed oligonucleotide, thereby producing an altered immunoglobulin variable domain coding sequence or complement thereof.

4. A method of selecting a nucleic acid encoding a protein, the method comprising:
a) providing a plurality of genetic packages, each package comprising an accessible protein that varies among the plurality of genetic packages and a coding nucleic acid that encodes the accessible protein;
b) selecting one or more packages of the plurality that display accessible proteins that have at least a threshold degree of a given activity;
c) preparing template nucleic acids from at least one of the one or more selected packages;
d) providing a plurality of diversity oligonucleotides that can anneal to at least some of the template nucleic acids;
e) combining the diversity oligonucleotides and the template nucleic acids in a mixture;
f) subjecting the mixture to conditions such that only a subset of the plurality of diversity oligonucleotides can anneal to the template nucleic acids; and
g) extending and/or ligating an annealed oligonucleotide of the subset to form a nucleic acid strand that is partially complementary to the template nucleic acid strand and that encodes a protein having a sequence altered relative to the protein selected for at least a threshold level of a given activity.

5. The method of claim 1, 2, 3, or 4, the subjecting comprises separating at least some members of the subset from the remaining diversity oligonucleotides of the plurality.

6. The method of claim 5 wherein the separating comprises washing the template nucleic acid strand.

7. The method of claim 5 wherein at least one template nucleic acid strand is covalently linked to a solid support.

8. The method of claim 1, 2, 3, or 4, wherein the diversity oligonucleotides of the plurality are between 30 and 90 nucleotides in length.

9. The method of claim 1, 2, 3, or 4 wherein each of the diversity oligonucleotides (1) is of equal length as the other diversity oligonucleotides or within 20% of the average of all the diversity oligonucleotide lengths, and/or (2) includes 3′ and 5′ terminal regions between 6 and 15 basepairs in length, the terminal regions being substantially identical to the corresponding terminal regions of each of the other diversity oligonucleotides.

10. The method of claim 9 wherein the 3′ and 5′ terminal regions are exactly complementary to a corresponding site on the template nucleic acid.

11. The method of claim 1, 2, 3, or 4, wherein each diversity oligonucleotide of the plurality of diversity oligonucleotides is a naturally occurring sequence.

12. The method of claim 1 or 2, wherein each diverse nucleic acid of the plurality of diverse nucleic acids comprises a sequence encoding an immunoglobulin variable domain or complement thereof.

13. The method of claim 12 wherein each diverse nucleic acid of the plurality of diverse nucleic acids is isolated from a hematopoictic cell that produces mature immunoglobulins.

14. The method of claim 2 wherein each template nucleic acid of the plurality of template nucleic acids comprises a sequence encoding a polypeptide of at least 20 amino acids.

15. The method-of claim 2 wherein template nucleic acid strands of the plurality differ from one another.

16. The method of claim 15 wherein the plurality of template nucleic acid strands comprises at least 10 different nucleic acids.

17. The method of claim 2 wherein each respective polypeptide encoded by plurality of template nucleic acids is preselected for at least one given activity.

18. The method of claim 17 wherein the given activity is binding to a target molecule or target cell.

19. The method of claim 3 wherein the diversity oligonucleotides comprise a first set of oligonucleotides that comprise a sequence encoding a first complementarity determining region (CDR), or complement thereof and a second set of oligonucleotides that comprise a sequence encoding a second complementarity determining region (CDR), or complement thereof.

20. The method of claim 19 wherein the first complementarity determining region is CDR1 and the second complementarity determining region is CDR2.

21. The method of claim 19 wherein step d) comprises extending and/or ligating an annealed oligonucleotide of the first set and an annealed oligonucleotide of the second set to form an altered nucleic acid strand that incorporates the annealed oligonucleotide of the first set, the annealed oligonucleotide of the second set, and a region complementary to the template nucleic acid strand.

22. The method of claim 1, 2, 3, or 4, wherein the plurality of diversity oligonucleotides comprises at least 10² different sequences.

23. The method of claim 3 wherein providing a plurality of diversity oligonucleotides comprises annealing a cleavage-directing oligonucleotide to each nucleic acid of a plurality of diverse nucleic acids to form a cleavable region on each of the diverse nucleic acids and cleaving the cleavable region of each diverse nucleic acid to form a plurality of diversity oligonucleotides, wherein the plurality of diverse nucleic acids are cDNAs prepared from an immune cell that produces an immunoglobulin protein.

24. The method of claim 1, 2, or 23 wherein the cleavage-directing oligonucleotide includes a stem-loop structure.

25. The method of claim 23 wherein the stem-loop structure comprises a recognition site for a Type IIS restriction enzyme and the cleaving is effected by the Type IIS restriction enzyme.

26. The method of claim 2 wherein the cleaving is effected by a Type II restriction enzyme.

27. The method of claim 26 wherein the Type II restriction enzyme recognizes a site of less than six basepairs.

28. The method of claim 2 wherein the cleaving is effected at a temperature greater than 40° C.

29. The method of claim 1, 2, or 23, wherein at least two cleavage-directing oligonucleotides are annealed to each of the diverse nucleic acid and cleaved.

30. The method of claim 29 wherein the at least two cleavage-directing oligonucleotides are cleaved sequentially.

31. The method of claim 2, wherein the cleavage-directing oligonucleotide is partially complementary to at least some of the diverse nucleic acids of the plurality.

32. The method of claim 4, wherein each genetic package is a replicable bacteriophage particle.

33. The method of claim 4, wherein each accessible protein comprises an immunoglobulin variable domain.

34. A method of providing a library of genetic packages that present an immunoglobulin protein, the method comprising:
a) providing a first plurality of genetic packages, each package comprising an accessible protein that comprises an immunoglobulin variable domain and varies among the plurality of genetic packages and a coding nucleic acid that encodes the accessible protein;
b) contacting the first plurality of genetic packages to a target;
c) separating genetic packages of the first plurality that bind to the target from genetic packages that do not bind to the target;
d) preparing template nucleic acids from at least one of the separated genetic packages that bind to the target, the template nucleic acids comprising a sequence from the coding nucleic acid of the respective genetic packages;
e) providing a plurality of diversity oligonucleotides that can anneal to at least some of the template nucleic acids and that each comprise a nucleic acid sequence encoding a single CDR and a portion of the flanking framework regions, or a complement thereof;
f) combining the diversity oligonucleotides and the template nucleic acids in a mixture;
g) subjecting the mixture to conditions such that only a subset of the plurality of diversity oligonucleotides can anneal to the template nucleic acids;
h) extending and/or ligating an annealed oligonucleotide of the subset to form a plurality of altered nucleic acid strands that each incorporate a diversity oligonucleotide and a sequence complementary to one of the template nucleic acids, and
i) preparing a second plurality of genetic packages from the altered nucleic acid strands or complements thereof as coding nucleic acids for the accessible protein component of each respective genetic package, thereby providing a library of genetic packages that present an immunoglobulin protein.

35. The method of claim 34 further comprising:
j) contacting the second plurality of genetic packages to a target; and
k) separating genetic package of the second plurality that bind to the target from genetic packages that do not bind to the target.

36. A library of genetic packages constructed by the method of claim 34.

37. The method of claim 2 further comprising g) constructing a library of nucleic acids from the diversity strands formed from each template nucleic acid strand of the plurality.

38. A library of nucleic acids constructed by the method of claim 37.

39. A method comprising:
a) providing a plurality of diverse subject nucleic acids, each being attached to an insoluble support and including a single-stranded region;
b) annealing a first oligonucleotide to each subject nucleic acid of the plurality to form first double-stranded segments;
c) cleaving the first double-stranded segments to release first fragments from the insoluble support and first-cleaved subject nucleic acids attached to the insoluble support;
d) annealing a second oligonucleotide to each of the first-cleaved subject nucleic acids to form second double-stranded segments;
e) cleaving the second double-stranded segments to release second fragments from the insoluble support and a second-cleaved subject nucleic acid attached to the insoluble support; and
f) recovering the second fragments from the insoluble support.

40. The method of claim 39 further comprising annealing at least one of the second fragments to a template nucleic acid and extending the annealed second fragment.

41. The method of claim 39 wherein the first and/or second oligonucleotide includes a double-stranded segment that is recognized by a Type IIS enzyme.

42. The method of claim 39 wherein each subject nucleic acid comprises a sequence encoding an immunoglobulin variable domain or fragment thereof that includes at least two CDRs, or a complement of the sequence, wherein each of the second fragments comprises a sequence encoding a single CDR or complement thereof.

43. The method of claim 4 wherein the accessible protein of each genetic package comprises an intra-molecular disulfide bond.

44. The method of claim 4 wherein the accessible protein of each genetic package comprises a varied region having fewer than 20 varied amino acid positions.

45. The method of claim 4 wherein the accessible protein of each genetic package comprises a modified scaffold domain that folds independently and is less than 90 amino acids in length.

46. The method of claim 45 wherein the varied region is less than 30 amino acid in length.

47. The method of claim 46 wherein the varied region comprises two invariant cysteine residues.

48. The method of claim 46 wherein the diversity oligonucleotides each comprise a nucleic acid sequence that encodes an amino acid sequence spanning the varied sequence or a complement thereof.

49. The method of claim 48 wherein the diversity oligonucleotides are synthesized using trinucleotide subunits.

50. A method of altering a nucleic acid sequence encoding a peptide, the method comprising:
a) providing i) one or more template nucleic acids, each encoding a peptide of less than 31 amino acids that independently binds to a target molecule, or complement thereof, and ii) a plurality of diversity oligonucleotides that can anneal to at least one of the one or more template nucleic acids at a site that overlaps a sequence encoding the peptide, or complement thereof, wherein the diversity oligonucleotides include at least 10³ different nucleic acids sequences;
b) combining the diversity oligonucleotides and the one or more template nucleic acids in a mixture;
c) subjecting the mixture to conditions such that only a subset of the plurality of diversity oligonucleotides can anneal to the one or more template nucleic acids;
d) extending and/or ligating an annealed oligonucleotide of the subset to form a plurality of altered nucleic acid strands that each incorporate a diversity oligonucleotide and a sequence complementary to one of the template nucleic acids.
